(19) United States
(12) Patent Application Publication
(10) Pub. No.: US 2007/0191335 A1
(43) Pub. Date: Aug. 16, 2007

(54) Heterocylic antiviral compounds

(76) Inventors: Remy Lemoine, San Francisco, CA (US); Chris Richard Melville, Palo Alto, CA (US); David Mark Rotstein, Sunnyvale, CA (US); Jutta Wanner, Mountain View, CA (US)

Correspondence Address: ROCHE PALO ALTO LLC;PATENT LAW DEPT. M/S A2-250, 3431 HILLVIEW AVENUE, PALO ALTO, CA, 94304, US

(21) Appl. No.: 11/706,807

(22) Filed: Feb. 15, 2007

Related U.S. Application Data
(60) Provisional application No. 60/773,942, filed on Feb. 15, 2006.

(51) Int. Cl. A61K 31/506; A61K 31/497; A61K 31/501; A61K 31/454; A61K 31/4439; C07D 487/02

(52) U.S. Cl. 514/210.21; 514/321; 514/412; 546/199; 514/453; 548/311.1; 514/397; 514/275; 514/338; 544/331; 546/277.1; 544/238; 514/255.05; 514/252.06; 544/405; 514/381; 548/253

(57) ABSTRACT

Chemokine receptor antagonists, in particular, 3,7-diazabicyclo[3.3.0]octane compounds according to formula (I) wherein R¹-R³ R^{6c} and X¹ are as defined herein are antagonists of chemokine CCR5 receptors which are useful for treating or preventing an human immunodeficiency virus (HIV-1) infection, or treating AIDS or ARC. The invention further provides methods for treating diseases that are alleviated with CCR5 antagonists. The invention includes pharmaceutical compositions and methods of using the compounds for the treating diseases mediated by the CCR5 receptor.

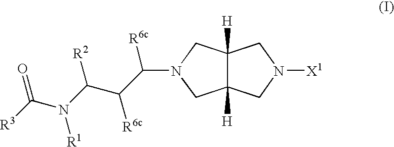

### CROSS REFERENCE TO PRIOR APPLICATIONS

This application claims the benefit of priority to U.S. Ser. No. 60/773,942 filed Feb. 15, 2005 the contents of which are hereby incorporated in their entirety by reference.

### FIELD OF THE INVENTION

This invention relates to octahydro-pyrrolo[3,4-c]pyrrole derivatives useful in the treatment of a variety of disorders, including those in which the modulation of CCR5 receptors is desirable. More particularly, the present invention relates to 3-(hexahydro-pyrrolo[3,4-c]pyrrol-2-yl)-1-phenyl-propylamine and [3-(hexahydro-pyrrolo[3,4-c]pyrrol-2-yl)-propyl]-phenyl-amine compounds and related derivatives, to compositions containing, to uses of such derivatives and to processes for preparing said compounds. Disorders that may be treated or prevented by the present derivatives include HIV-1 and HIV-1-mediated retroviral infections (and the resulting acquired immune deficiency syndrome, AIDS), diseases of the immune system and inflammatory diseases.

### BACKGROUND OF THE INVENTION

A-M. Vandamme et al. (Antiviral Chemistry & Chemotherapy, 1998 9:187-203) disclose current HAART clinical treatments of HIV-1 infections in man including at least triple drug combinations. Highly active anti-retroviral therapy (HAART) has traditionally consisted of combination therapy with nucleoside reverse transcriptase inhibitors (NRTI), non-nucleoside reverse transcriptase inhibitors (NNRTI) and protease inhibitors (PI). These compounds inhibit biochemical processes required for viral replication. In compliant drug-naive patients, HAART is effective in reducing mortality and progression of HIV-1 to AIDS. While HAART has dramatically altered the prognosis for HIV-1 infected persons, there remain many drawbacks to the current therapy including highly complex dosing regimes and side effects which can be very severe (A. Carr and D. A. Cooper, Lancet 2000 356(9239):1423-1430). Moreover, these multidrug therapies do not eliminate HIV-1 and long-term treatment usually results in multidrug resistance, thus limiting their utility in long term therapy. Development of new drug therapies to provide better HIV-1 treatment remains a priority.

The chemokines are a large family of pro-inflammatory peptides that exert their pharmacological effect through G-protein-coupled receptors. The CCR5 receptor is one member of this family. The chemokines are leukocyte chemotactic proteins capable of attracting leukocytes to various tissues, which is an essential response to inflammation and infection. The name “chemokine”, is a contraction of “chemotactic cytokines”. Human chemokines include approximately 50 structurally homologous small proteins comprising 50-120 amino acids. (M. Baggiolini et al., Ann. Rev. Immunol. 1997 15:675-705)

The CCR5 receptor is a chemokine receptor. The chemokines are a subset of the cytokine family of soluble immune mediators. Chemokine receptors are seven membrane-spanning receptors that signal through heterotrimeric G protein when bound to an agonist. Human CCR5 is composed of 352 amino acids with an intra-cellular C-terminus containing structural motifs for G-protein association and ligand-dependent signaling (M. Oppermann Cellular Signaling 2004 16:1201-1210). The extracellular N-terminal domain contributes to high-affinity chemokine binding and interactions with the gp120 HIV protein (T. Dragic J. Gen. Virol. 2001 82:1807-1814; C. Blanpain et al. J. Biol. Chem. 1999 274:34719-34727). The binding site for the natural agonist RANTES (Regulated upon Activation and is Normal T-cell Expressed and Secreted) has been shown to be on the N-terminal domain and HIV-1 gp120 has been suggested to interact initially with the N-terminal domain and also with the ECL2. (B. Lee, et al. J. Biol. Chem. 1999274:9617-26)]

Modulators of the CCR5 receptor may be useful in the treatment of various inflammatory diseases and conditions, and in the treatment of infection by HIV-1 and genetically related retroviruses. As leukocyte chemotactic factors, chemokines play an indispensable role in the attraction of leukocytes to various tissues of the body, a process which is essential for both inflammation and the body's response to infection. Because chemokines and their receptors are central to the pathophysiology of inflammatory, autoimmune and infectious diseases, agents which are active in modulating, preferably antagonizing, the activity of chemokines and their receptors, are useful in the therapeutic treatment of these diseases. The CCR5 receptor is of particular importance in the context of treating inflammatory and infectious diseases. The natural ligands for the CCR5 are the macrophage inflammatory proteins (MIP) designated MIP-1a and MIP-1b and RANTES.

HIV-1 infects cells of the monocyte-macrophage lineage and helper T-cell lymphocytes by exploiting a high affinity interaction of the viral enveloped glycoprotein (Env) with the CD4 antigen. The CD4 antigen, however appeared to be a necessary, but not sufficient requirement for cell entry and at least one other surface protein was required to infect the cells (E. A. Berger et al., Ann. Rev. Immunol. 1999 17:657-700). Two chemokine receptors, either the CCR5 or the CXCR4 receptor were subsequently found to be co-receptors which are required, along with CD4, for infection of cells by the human immunodeficiency virus (HIV). The central role of CCR5 in the pathogenesis of HIV was inferred by epidemiological identification of powerful disease modifying effects of the naturally occurring null allele CCR5 Δ32. The Δ32 mutation has a 32-base pair deletion in the CCR5 gene resulting in a truncated protein designated Δ32. Relative to the general population, Δ32/Δ32 homozygotes are significantly common in exposed/uninfected individuals suggesting the role of CCR5 in HIV cell entry (R. Liu et al., Cell 1996 86(3):367-377; M. Samson et al., Nature 1996 382(6593):722-725).

The HIV-1 envelope protein is comprised of two subunits: gp120, the surface subunit and gp41, the transmembrane subunit. The two subunits are non-covalently associated and form homotrimers which compose the HIV envelope. Each gp41 subunit contains two helical heptad repeat regions, HR1 and HR2 and a hydrophobic fusion region on the C-terminus.

The CD4 binding site on the gp120 of HIV appears to interact with the CD4 molecule on the cell surface inducing a conformation change in gp120 which creates or exposes a cryptic CCR5 (or CXCR4) binding site, and undergoes conformational changes which permits binding of gp120 to the CCR5 and/or CXCR4 cell-surface receptor. The bivalent interaction brings the virus membrane into close proximity with the target cell membrane and the hydrophobic fusion region can insert into the target cell membrane. A conformation change in gp41 creates a contact between the outer leaflet of the target cell membrane and the viral membrane which produces a fusion pore whereby viral core containing genomic RNA enters the cytoplasm

Viral fusion and cell entry is a complex multi-step process and each step affords the potential for therapeutic intervention. These steps include (i) CD40-gp120 interactions, (ii) CCR5 and/or CXCR-4 interactions and (iii) gp41 mediated membrane fusion. Conformational changes induced by these steps expose additional targets for chemotherapeutic intervention. Each of these steps affords an opportunity for therapeutic intervention in preventing or slowing HIV infection. Small molecules (Q. Guo et al. J. Virol. 2003 77:10528-63) and antibodies (D. R. Kuritzkes et al. 10^{th} Conference on Retroviruses and Opportunistic Infections, Feb. 10-14, 2003, Boston, Mass. Abstract 13; K. A. Nagashima et al. J. Infect. Dis. 2001 183:1121-25) designed to prevent the gp120/CD4 interaction have been disclosed. Small molecule antagonists of, and antibodies to, CCR5 are discussed below. A small molecular weight antagonist of CXCR4 has been explored (J. Blanco et al. Antimicrob. Agents Chemother. 2000 46:1336-39). Enfuvirtide (T20, ENF or FUZEON®) is a 36 amino acid peptide corresponding to residues 643-678 in the HR2 domain of gp41. Enfuvirtide binds to the trimeric coiled-coil by the HR1 domains and acts in a dominant negative manner to block the endogenous six helix bundle formation thus inhibiting viral fusion. (J. M. Kilby et al., New Eng. J. Med. 1998 4(11):1302-1307). Enfuvirtide has been approved for clinical use.

In addition to the potential for CCR5 modulators in the management of HIV infections, the CCR5 receptor is an important regulator of immune function and compounds of the present invention may prove valuable in the treatment of disorders of the immune system. Treatment of solid organ transplant rejection, graft v. host disease, arthritis, rheumatoid arthritis, inflammatory bowel disease, atopic dermatitis, psoriasis, asthma, allergies or multiple sclerosis by administering to a human in need of such treatment an effective amount of a CCR5 antagonist compound of the present invention is also possible. (M. A. Cascieri and M. S. Springer, Curr. Opin. Chem. Biol. 2000 4:420-427; A. Proudfoot et al., Immunol. Rev. 2000 177:246-256; P. Houshmand and A. Zlotnik, Curr. Opin. Chem. Biol. 2003 7:457-460)

Related octahydro-pyrrolo[3,4-c]pyrrole compounds which antagonists of the CCR5 receptor have been disclosed by E. K. Lee et al. in WO 2005121145 entitled Preparation of heterocyclic antiviral compounds, particularly (3-hexahydropyrrolo[3,4-c]pyrrol-2-yl)-1-phenylpropylamine and [3-(hexahydropyrrolo[3,4-c]pyrrol-2-yl)propyl]phenylamine derivatives as antagonists of chemokine CCR5 receptor, useful for treating HIV and genetically related retroviral infections, published Dec. 22, 2005 which is hereby incorporated by reference in its entirety

### SUMMARY OF THE INVENTION

The present invention relates to a compounds according to formula I which are CCR5 receptor antagonists, methods for treating diseases alleviated by administration of a compound according to formula I and pharmaceutical compositions for treating diseases containing a compound according to formula I admixed with at least one carrier, diluent or excipient, 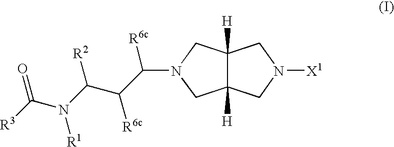
one of R¹ and R² is phenyl optionally substituted with one to four substitutents selected independently in each incidence from the group consisting of halogen, C₁₋₆ alkyl and C₁₋₆ alkoxy; and, the other of R¹ and R² is hydrogen;
R³ is selected from the group consisting of:
   (a) C₃₋₇ cycloalkyl optionally substituted with 1 to 4 fluorines, cyano, hydroxyl, C₁₋₃ alkyl or phenyl, 4-oxo-cyclohexyl or 3-oxo-cyclobutyl;
   (b) heterocycle selected from the group consisting of IIa-c, oxetanyl and tetrahydrofuranyl, 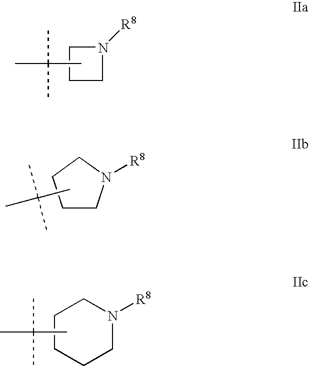
      wherein:
         R⁸ is hydrogen, COR⁹, COCHR¹⁴NHR¹⁵ or SO₂R¹⁰; and,
         R⁹ is C₁₋₆ alkyl or C₃₋₇ cycloalkyl;
         R¹⁰ is C₁₋₆ alkyl;
         R¹⁴ is the side chain of naturally occurring amino acid;
         R¹⁵ is hydrogen, tert-butoxycarbonyl or benzyloxycarbonyl;
   (c) C₁₋₆ alkyl; and
   (d) C₁₋₆ alkoxy;
R⁶ is hydrogen, C₁₋₃ alkyl, C₁₋₃ haloalkyl, C₁₋₆ hydroxyalkyl or oxo-C₁₋₆ alkyl;
R^{6a}, R^{6b}, R^{6c} and R^{6d} are independently hydrogen or C₁₋₃ alkyl with the proviso that at least one of R^{6c} is hydrogen;
X¹ is selected from the group consisting of (i)-(ix) and (x) wherein:
   X² is N or CH;
   X^{2a} is N, CH or CCl;
   A¹ is phenylene or C₁₋₆ straight or branched alkylene optionally substituted by a phenyl ring;
   R⁵ is hydroxy, C₁₋₆ alkoxy, benzyloxy or NR^{6a}R^{6b}; 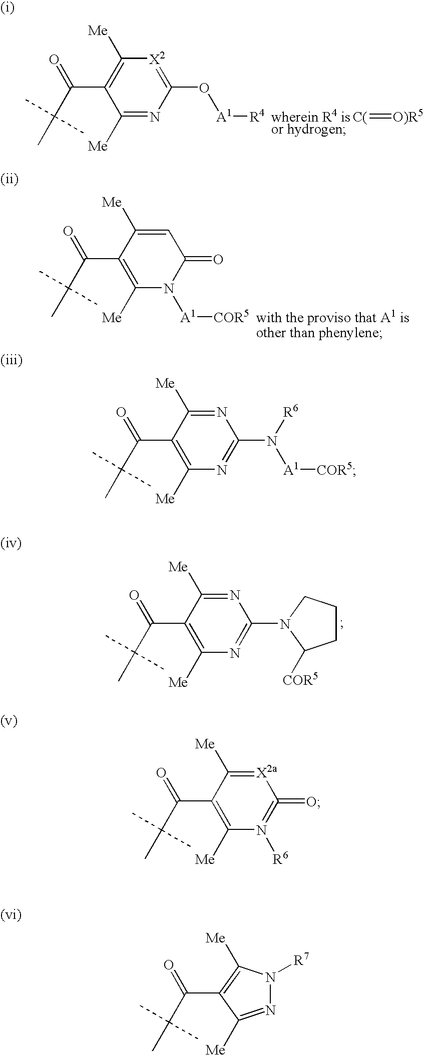
      wherein:
         R⁷ is C₃₋₇ cycloalkyl, (CH₂)ₙCOR⁵, heteroaryl selected from the group consisting of pyridine, pyrimidine, pyrazine and pyridazine said heteroaryl optionally substituted with C₁₋₃ alkyl or C₁₋₃ haloalkyl;
         n is 1 to 3; 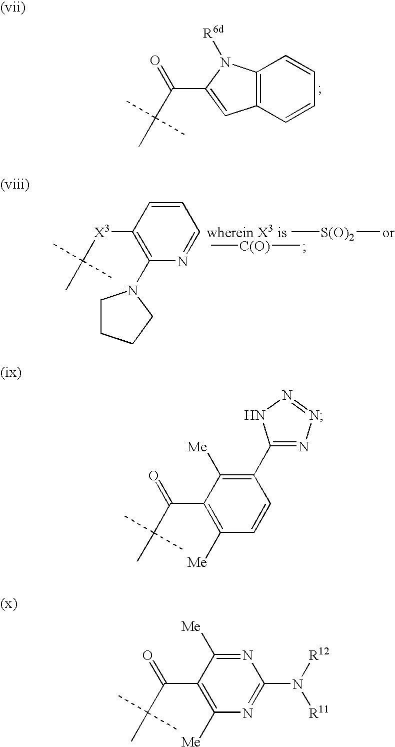
      wherein
         R¹¹ and R¹² are (A) together a group (CH₂)₂X⁴(CH₂)₂, (CH₂)₂CH(R¹⁶)CH₂, (CH₂)₂SO₂, or (B) independently R¹² is hydrogen or C₁₋₃ alkyl and R¹¹ is —SO₂C₁₋₆ alkyl, C₁₋₆ hydroxyalkyl, xA, xB or xC; 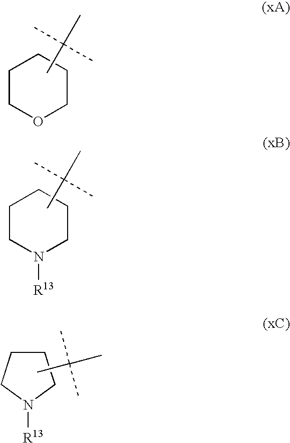
         X⁴ is O, S(O)ₘ, NR¹³ or CH(NHSO₂C₁₋₆ alkyl);
         R¹³ is R^{6d}, —C(O)C₁₋₆ alkyl, S(O)₂C₁₋₆ alkyl;
         R¹⁶ is hydrogen, hydroxyl or C₁₋₁₀ acyloxy;
         m is zero to two; and, 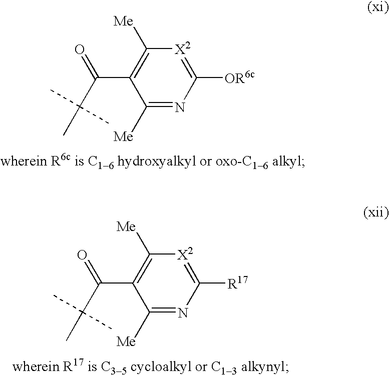
   or pharmaceutically acceptable salts, hydrates or solvates thereof.

### DETAILED DESCRIPTION OF THE INVENTION

In one embodiment of the present invention there is provided a compound according to formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, R¹⁴, R¹⁵, R¹⁶, R¹⁷, X¹, X², X^{2a}, X³, X⁴, A¹ and m are as described herein above. In this and the following embodiments, any substitutent not explicitly defined, retains the broadest definition provided in the summary of the invention or in claim 1.

In another embodiment of the present invention there is provided a compound according to formula I wherein R⁶ is hydrogen or C₁₋₃ alkyl; X^{2a} is N or CH; X¹ is selected from the group consisting of (i) to (ix) and (x) and when X¹ is (x), R¹¹ and R¹² are (A) together a group (CH₂)₂X⁴(CH₂)₂ or (B) independently R¹² is hydrogen or C₁₋₃ alkyl and R¹¹ is —SO₂C₁₋₆ alkyl, xA, xB; X⁴ is O, S(O)ₘ or NR¹³; and R¹, R², R³, R⁴, R⁵, R^{6a}, R^{6b}, R^{6c}, R^{6d}, R⁷, R⁸, R⁹, R¹⁰, R¹³, R¹⁴, R¹⁵, X², X³, X⁴, A¹ and m are as described herein above.

In yet another embodiment of the present invention there is provided a compound according to formula I wherein R¹ is hydrogen; R² is phenyl optionally substituted with chlorine or fluorine; R³ is (a) C₃₋₇ cycloalkyl optionally substituted with one to four fluorines, 4-oxo-cyclohexyl or 3-oxo-cyclobutyl, (b) heterocycle selected from the group consisting of IIa, IIc and tetrahydrofuranyl (wherein R⁸ is COR⁹ and, R⁹ is C₁₋₆ alkyl or C₃₋₇ cycloalkyl) (c) C₁₋₆ alkyl, or (d) C₁₋₆ alkoxy; R^{6c} in each occurrence is hydrogen; X¹ is selected from (i), (iii), (v) and (vi) (wherein (i), (iii), (v) and (vi) are as described in claim 1; A¹ is C₁₋₆ straight or branched alkylene; R⁷ is C₃₋₇ cycloalkyl or heteroaryl selected from the group consisting of pyridine, pyrimidine, pyrazine and pyridazine said heteroaryl optionally substituted with C₁₋₃ alkyl or C₁₋₃ haloalkyl) and X^{2a}, is N or CH.

In another embodiment of the present invention there is provided a compound according to formula I wherein R¹ is hydrogen; R² is phenyl optionally substituted with chlorine or fluorine; R³ is (a) C₃₋₇ cycloalkyl optionally substituted with one to four fluorines, 4-oxo-cyclohexyl or 3-oxo-cyclobutyl or (b) heterocycle selected from the group consisting of IIa (wherein R⁸ is COR⁹ and R⁹ is C₁₋₆ alkyl or C₃₋₇ cycloalkyl) or tetrahydrofuranyl, R^{6c} in each occurrence is hydrogen; X¹ is (vi) (wherein (vi) is as defined in claim 1 and R⁷ is heteroaryl selected from the group consisting of pyridine, pyrimidine, pyrazine and pyridazine said heteroaryl optionally substituted with C₁₋₃ alkyl or C₁₋₃ haloalkyl).

In another embodiment of the present invention there is provided a compound according to formula I wherein R¹ is hydrogen; R² is phenyl optionally substituted with chlorine or fluorine; R³ is (a) C₃₋₇ cycloalkyl optionally substituted with one to four fluorines, 4-oxo-cyclohexyl or 3-oxo-cyclobutyl or (b) heterocycle selected from the group consisting of IIa (wherein R⁸ is COR⁹ and R⁹ is C₁₋₆ alkyl or C₃₋₇ cycloalkyl) or tetrahydrofuranyl; R^{6c} in each occurrence is hydrogen; X¹ is (v) as defined in claim 1 (wherein R⁶ is C₁₋₆ alkyl) and X^{2a} is N or CH.

In another embodiment of the present invention there is provided a compound according to formula I wherein R¹ is hydrogen; R² is phenyl optionally substituted with chlorine or fluorine; R³ is C₃₋₇ cycloalkyl optionally substituted with one to four fluorines, 4-oxo-cyclohexyl or 3-oxo-cyclobutyl; R^{6c} in each occurrence is hydrogen; X¹ is (i) or (iii) as defined in claim 1 (wherein R⁵ is hydroxy or C₁₋₆ alkoxy).

In another embodiment of the present invention there is provided a compound according to formula I wherein R¹ is phenyl optionally substituted with one to four substitutents selected independently in each incidence from the group consisting of halogen and C₁₋₆ alkyl; R² is hydrogen; R³ is (a) C₃₋₇ cycloalkyl optionally substituted with one to four fluorines, 4-oxo-cyclohexyl or 3-oxo-cyclobutyl, (b) heterocycle selected from the group consisting of IIa, IIc, oxetanyl and tetrahydrofuranyl (wherein R⁸ is COR⁹ and R⁹ is C₁₋₆ alkyl or C₃₋₇ cycloalkyl), (c) C₁₋₆ alkyl or (d) C₁₋₆ alkoxy; R^{6c} in each occurrence is hydrogen; X¹ is (i), (iii), (v) or (vi) as defined in claim 1 (wherein A¹ is C₁₋₆ straight or branched alkylene and R⁷ is C₃₋₇ cycloalkyl or heteroaryl selected from the group consisting of pyridine, pyrimidine, pyrazine and pyridazine said heteroaryl optionally substituted with C₁₋₃ alkyl or C₁₋₃ haloalkyl) and X^{2a} is N or CH.

In another embodiment of the present invention there is provided a compound according to formula I wherein R¹ is phenyl optionally substituted with one to four substitutents selected independently in each incidence from the group consisting of halogen and C₁₋₆ alkyl; R² is hydrogen; R³ is (a) C₃₋₇ cycloalkyl optionally substituted with one to four fluorines, 4-oxo-cyclohexyl or 3-oxo-cyclobutyl or (b) IIc (wherein R⁸ is COR⁹ and R⁹ is C₁₋₆ alkyl), X¹ is (i) or (v) as defined in claim 1 (wherein X² is CH and R⁵ is hydroxy or C₁₋₆ alkoxy) and X^{2a} is N or CH.

In another embodiment of the present invention there is provided a compound according to formula I wherein R¹ is phenyl optionally substituted with one to four substitutents selected independently in each incidence from the group consisting of halogen and C₁₋₆ alkyl; R² is hydrogen; R³ is (a) C₃₋₇ cycloalkyl optionally substituted with one to four fluorines, 4-oxo-cyclohexyl or 3-oxo-cyclobutyl or (b) IIc (wherein R⁸ is COR⁹ and R⁹ is C₁₋₆ alkyl), X¹ is (iii) as defined in claim 1 (wherein R⁵ is hydroxy or C₁₋₆ alkoxy).

In another embodiment of the present invention there is provided a compound according to formula I wherein R¹ is hydrogen; R² is phenyl optionally substituted with chlorine or fluorine; R³ is C₃₋₇ cycloalkyl optionally substituted with one to four fluorines, 4-oxo-cyclohexyl or 3-oxo-cyclobutyl, R^{6c} in each occurrence is hydrogen; X¹ is (x) wherein x is as defined in claim 1 (wherein R¹¹ is SO₂C₁₋₆ alkyl and R¹² is hydrogen or C₁₋₃ alkyl). In this embodiment, any other embedded substitutents not explicitly limited retain the definition provided in the summary of the invention.

In another embodiment of the present invention there is provided a compound according to formula I wherein R¹ is hydrogen; R² is phenyl optionally substituted with chlorine or fluorine; R³ is C₃₋₇ cycloalkyl optionally substituted with one to four fluorines, 4-oxo-cyclohexyl or 3-oxo-cyclobutyl, R^{6c} in each occurrence is hydrogen; X¹ is (x) wherein x is as defined in claim 1 (wherein (A) R¹¹ and R¹² together are (CH₂)₂X⁴(CH₂)₂, X⁴ is O or NR¹³ and R¹³ is C(O)C₁₋₆ alkyl or (B) R¹¹ is 4-tetrahydropyran-4-yl and R¹² is hydrogen). In this embodiment, any other embedded substitutents not explicitly limited retain the definition provided in the summary of the invention.

In another embodiment of the present invention there is provided a compound according to formula I which compound is selected from compounds I-1 to I-52 of TABLE 1, II-1 to II-29 of TABLE 2 and III-1 to III-28 of TABLE 3.

In another embodiment of the present invention there is provided a method for treating or preventing an human immunodeficiency virus (HIV) infection, or treating AIDS or ARC, in a patient in need thereof which comprises administering to the patient a therapeutically effective amount of a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, R¹⁴, R¹⁵, R¹⁶, R¹⁷, X¹, X², X^{2a}, X³, X⁴, A¹ and m are as described herein above.

In another embodiment of the present invention there is provided a method for treating or preventing an human immunodeficiency virus (HIV) infection, or treating AIDS or ARC, in a patient in need thereof which comprises co-administering a therapeutically effective amount of at least one compound selected from the group consisting of HIV nucleoside reverse transcriptase inhibitors, HIV non-nucleoside reverse transcriptase inhibitors, HIV protease inhibitors and viral fusion inhibitors in additional to a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, R¹⁴, R¹⁵, R¹⁶, R¹⁷, X¹, X², X^{2a}, X³, X⁴, A¹ and m are as described herein above.

In another embodiment of the present invention there is provided a method for treating or preventing an human immunodeficiency virus (HIV) infection, or treating AIDS or ARC, in a patient in need thereof which comprises co-administering a therapeutically effective amount of at least one of efavirenz, nevirapine, delavirdine, zidovudine, didanosin, zalcitabine, stavudine, lamivudine, abacavir, adefovir and dipivoxil, saquinavir, ritonavir, nelfinavir, indinavir, amprenavir, lopinavir or T-20 in additional to a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, R¹⁴, R¹⁵, R¹⁶, R¹⁷, X¹, X², X^{2a}, X³, X⁴, A¹ and m are as described herein above.

In another embodiment of the present invention there is provided a method for treating a mammal with a disease state that is alleviated by a CCR5 receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis which comprises administering to the mammal in need thereof a therapeutically effective amount of a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, R¹⁴, R¹⁵, R¹⁶, R¹⁷, X¹, X², X^{2a}, X³, X⁴, A¹ and m are as described herein above.

In another embodiment of the present invention there is provided a method for treating a mammal with a disease state that is alleviated by a CCR5 receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis which comprises co-administering to the mammal in need thereof a therapeutically effective amount at least one other immune modulator and a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, R¹⁴, R¹⁵, R¹⁶, R¹⁷, X¹, X², X^{2a}, X³, X⁴, A¹ and m are as described herein above.

In another embodiment of the present invention there is provided a method for treating a human with a disease state that is alleviated by a CCR5 receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis which comprises co-administering to the mammal in need thereof a therapeutically effective amount at least one other immune modulator and a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, R¹⁴, R¹⁵, R¹⁶, R¹⁷, X¹, X², X^{2a}, X³, X⁴, A¹ and m are as described herein above.

In another embodiment of the present invention there is provided a pharmaceutical composition for treating or preventing an human immunodeficiency virus (HIV-1) infection, or treating AIDS or ARC comprising a compound according to formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, R¹⁴, R¹⁵, R¹⁶, R¹⁷, X¹, X², X^{2a}, X³, X⁴, A¹ and m are as described herein above said compound of formula I admixed with at least one pharmaceutical acceptable carrier, diluent or excipient.

In another embodiment of the present invention there is provided a pharmaceutical composition for treating a mammal with a disease state that is alleviated by a CCR5 receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis comprising a compound according to formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, R¹⁴, R¹⁵, R¹⁶, R¹⁷, X¹, X², X^{2a}, X³, X⁴, A¹ and m are as described herein above said compound of formula I admixed with at least one pharmaceutical acceptable carrier, diluent or excipient.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the first definition provided in the Summary of the Invention.

The term “optional” or “optionally” as used herein means that a subsequently described event or circumstance may, but need not, occur, and that the description includes instances where the event or circumstance occurs and instances in which it does not. For example, “optionally substituted” means that the moiety may be hydrogen or a substitutent.

It is contemplated that the definitions described herein may be appended to form chemically-relevant combinations, such as “heteroalkylaryl,” “haloalkylheteroaryl,” “arylalkylheterocyclyl,” “alkylcarbonyl,”“alkoxyalkyl,” and the like.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. One or more of the carbon atoms may optionally be replaced by oxygen, sulfur, substituted or unsubstituted nitrogen atom(s). Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substitutents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical. An “alkylamino alkyl” is an alkyl group having one to two alkylamino substitutents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below.

The term “alkylene” as used herein denotes a divalent saturated linear hydrocarbon radical of 1 to 6 carbon atoms (e.g., (CH₂)ₙ) or a branched saturated divalent hydrocarbon radical of 2 to 6 carbon atoms (e.g., —CHMe— or —CH₂CH(i-Pr)CH₂—), unless otherwise indicated. The open valences of an alkylene group are not attached to the same atom. Examples of alkylene radicals include, but are not limited to, methylene, ethylene, propylene, 2-methyl-propylene, butylene, 2-ethylbutylene.

The term “haloalkyl” as used herein denotes a unbranched or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, difluoromethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl.

The term “cyano” as used herein refers to a carbon linked to a nitrogen by a triple bond, i.e., —C≡N.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term “arylcarbonyl” as used herein means a group of formula C(═O)R wherein R is an aryl group; the term “benzoyl” as used herein an “arylcarbonyl” group wherein R is phenyl.

The term “acyloxy” as used herein denotes the radical —OC(O)R, wherein R is a lower alkyl radical as defined herein. Examples of acyloxy radicals include, but are not limited to, acetoxy, propionyloxy.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “halogen” or “halo” as used herein means fluorine, chlorine, bromine, or iodine.

The term “aryl” as used herein denotes a monovalent aromatic carbocyclic radical containing 5 to 15 carbon atoms consisting of one individual ring, or one or more fused rings in which at least one ring is aromatic in nature, which can optionally be substituted with one or more, preferably one or three substitutents independently selected from hydroxy, thio, cyano, alkyl, alkoxy, lower haloalkoxy, alkylthio, halogen, haloalkyl, hydroxyalkyl, nitro, alkoxycarbonyl, amino, alkylamino, dialkylamino, amino alkyl, alkylamino alkyl, and dialkylamino alkyl, alkylsulfonyl, arylsulfinyl, alkylaminosulfonyl, arylaminosulfonyl, alkylsulfonylamino, arylsulfonylamino, carbamoyl, alkylcarbamoyl and dialkylcarbamoyl, arylcarbamoyl, alkylcarbonylamino, arylcarbonylamino, unless otherwise indicated. Alternatively two adjacent atoms of the aryl ring may be substituted with a methylenedioxy or ethylenedioxy group. Thus a bicyclic aryl substitutents may be fused to a heterocyclyl or heteroaryl ring; however, the point of attachment of bicyclic aryl substitutent is on the carbocyclic aromatic ring. Examples of aryl radicals include, phenyl, naphthyl, indanyl, anthraquinolyl, tetrahydronaphthyl, 3,4-methylenedioxyphenyl, 1,2,3,4-tetrahydroquinolin-7-yl, 1,2,3,4-tetrahydroisoquinoline-7-yl, and the like. The term “phenylene” refers to a divalent phenyl ring which can by o-, m- or p-phenylene.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on a heteroaryl ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazolyl, isoxazolyl, thiazolyl, isothiazolyl, triazolinyl, thiadiazolyl and oxadiaxolinyl which can optionally be substituted with one or more, preferably one or two substitutents selected from hydroxy, cyano, alkyl, alkoxy, thio, lower haloalkoxy, alkylthio, halo, haloalkyl, alkylsulfinyl, alkylsulfonyl, halogen, amino, alkylamino, dialkylamino, amino alkyl, alkylamino alkyl, and dialkylamino alkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl” or “heterocycle” as used herein denotes a monovalent saturated cyclic radical, consisting of one or more rings, preferably one to two rings, of three to eight atoms per ring, incorporating one or more ring heteroatoms (chosen from N,O or S(O)0-2), and which can optionally be independently substituted with one or more, preferably one or two substitutents selected from hydroxy, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. A bicyclic heterocycle can be fused to an aryl or heteroaryl ring; however, the point of attachment is on the heterocyclic ring. Examples of heterocyclic radicals include, but are not limited to, azetidinyl, pyrrolidinyl, hexahydro azepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

The term “cycloalkyl” as used herein denotes a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term “oxetane” refers to a four-membered saturated heterocycle containing one oxygen atom. “Oxetanyl” refers to an oxetane radical.

The terms “hydroxyalkyl” as used herein denotes the radical R′R″ where R′ is a hydroxy radical and R″ is as defined herein and the attachment point of the hydroxyalkyl radical will be on the alkylene radical.

The terms “oxo-C₁₋₆ alkyl” as used herein denotes C₁₋₆ alkyl radical as herein defined wherein two hydrogen atoms on the same carbon atom are replaced by an oxygen.

Amino acids comprise a carbon atom bonded to a carboxyl group, an amino group, a hydrogen atom and a unique “side chain” group. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine and lysine. The side chains of naturally occurring amino acids include: hydrogen, methyl, iso-propyl, iso-butyl, sec-butyl, —CH₂OH, —CH(OH)CH₃, —CH₂SH, —CH₂CH₂SMe, —(CH₂)ₚCOR wherein R is —OH or —NH₂ and p is 1 or 2, —(CH₂)_{q}—NH₂ where q is 3 or 4, —(CH₂)₃—NHC(═NH)NH₂, —CH₂C₆H₅, —CH₂-p-C₆H₄—OH, (3-indolinyl)methylene, (4-imidazolyl)methylene.

Compounds of formula I exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

The term “protecting group” (PG) as used herein refers to a chemical group that (a) efficiently combines with a reactive group in a molecule; (b) prevents a reactive group from participating in an undesirable chemical reaction; and (c) can be easily removed after protection of the reactive group is no longer required. Protecting groups are used in synthesis to temporarily mask the characteristic chemistry of a functional group because it interferes with another reaction. Reagents and protocols for to introduce and remove protecting groups are well known and have been reviewed in numerous texts (e.g., T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 John Wiley and Sons, 1971-1996). One skilled in the chemical arts will appreciate that on occasion protocols must be optimized for a particular molecule and such optimization is well with the ability of one skilled in these arts. Amino-protecting groups used extensively herein include N-urethanes such as the N-benzyloxycarbonyl group (cbz) or tert-butoxycarbonyl (BOC) which is prepared by reaction with di(t-butyl)dicarbonate and benzyl groups. Benzyl groups are removed conveniently by hydrogenolysis and BOC groups are labile under acidic conditions.

It will be appreciated by the skilled artisan that the compounds of formula I may contain one or more chiral centers and therefore exist in two or more stereoisomeric forms. The racemates of these isomers, the individual isomers and mixtures enriched in one enantiomer, as well as diastereomers when there are two chiral centers, and mixtures partially enriched with specific diastereomers are within the scope of the present invention. It will be further appreciated by the skilled artisan that substitution of the tropane ring can be in either endo- or exo-configuration, and the present invention covers both configurations. The present invention includes all the individual stereoisomers (e.g. enantiomers), racemic mixtures or partially resolved mixtures of the compounds of formulae I and, where appropriate, the individual tautomeric forms thereof.

The racemates can be used as such or can be resolved into their individual isomers. The resolution can afford stereochemically pure compounds or mixtures enriched in one or more isomers. Methods for separation of isomers are well known (cf. Allinger N. L. and Eliel E. L. in “Topics in Stereochemistry”, Vol. 6, Wiley Interscience, 1971) and include physical methods such as chromatography using a chiral adsorbent. Individual isomers can be prepared in chiral form from chiral precursors. Alternatively individual isomers can be separated chemically from a mixture by forming diasteromeric salts with a chiral acid, such as the individual enantiomers of 10-camphorsulfonic acid, camphoric acid, .alpha.-bromocamphoric acid, tartaric acid, diacetyltartaric acid, malic acid, pyrrolidone-5-carboxylic acid, and the like, fractionally crystallizing the salts, and then freeing one or both of the resolved bases, optionally repeating the process, so as obtain either or both substantially free of the other; i.e., in a form having an optical purity of >95%. Alternatively the racemates can be covalently linked to a chiral compound (auxiliary) to produce diastereomers which can be separated by chromatography or by fractional crystallization after which time the chiral auxiliary is chemically removed to afford the pure enantiomers.

The compounds of formula I contain at least one basic center and suitable acid addition salts are formed from acids which form non-toxic salts. Examples of salts of inorganic acids include the hydrochloride, hydrobromide, hydroiodide, chloride, bromide, iodide, sulphate, bisulphate, nitrate, phosphate, hydrogen phosphate. Examples of salts of organic acids include acetate, fumarate, pamoate, aspartate, besylate, carbonate, bicarbonate, camsylate, D and L-lactate, D and L-tartrate, esylate, mesylate, malonate, orotate, gluceptate, methylsulphate, stearate, glucuronate, 2-napsylate, tosylate, hibenzate, nicotinate, isethionate, malate, maleate, citrate, gluconate, succinate, saccharate, benzoate, esylate, and pamoate salts. For a review on suitable salts see Berge et al., J. Pharm. Sci., 66, 1-19, 1977.

The term “solvate” as used herein means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “hydrate” as used herein means a compound of the invention or a salt thereof, that further includes a stoichiometric or non-stoichiometric amount of water bound by non-covalent intermolecular forces.

The term “clathrate” as used herein means a compound of the invention or a salt thereof in the form of a crystal lattice that contains spaces (e.g., channels) that have a guest molecule (e.g., a solvent or water) trapped within.

The term “nucleoside and nucleotide reverse transcriptase inhibitors” (“NRTI”s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA. Typical suitable NRTIs include zidovudine (AZT) available as RETROVIR® from Glaxo-Wellcome Inc.; didanosine (ddl) available as VIDEX® from Bristol-Myers Squibb Co.; zalcitabine (ddC) available as HIVID® from Roche Pharmaceuticals; stavudine (d4T) available as ZERIT® from Bristol-Myers Squibb Co.; lamivudine (3TC) available as EPIVIR® from Glaxo-Wellcome; abacavir (1592U89) disclosed in WO96/30025 and available ZIAGEN® from Glaxo-Wellcome; adefovir dipivoxil [bis(POM)-PMEA] available as PREVON® from Gilead Sciences; lobucavir (BMS-180194), a nucleoside reverse transcriptase inhibitor disclosed in EP-0358154 and EP-0736533 and under development by Bristol-Myers Squibb; BCH-10652, a reverse transcriptase inhibitor (in the form of a racemic mixture of BCH-10618 and BCH-10619) under development by Biochem Pharma; emitricitabine [(−)-FTC] licensed from Emory University under U.S. Pat. No. 5,814,639 and under development by Triangle Pharmaceuticals; beta-L-FD4 (also called beta-L-D4C and named beta-L-2′,3′-dicleoxy-5-fluoro-cytidene) licensed by Yale University to Vion Pharmaceuticals; DAPD, the purine nucleoside, (−)-b-D-2,6-diamino-purine dioxolane disclosed in EP-0656778 and licensed by Emory University and the University of Georgia to Triangle Pharmaceuticals; and lodenosine (FddA), 9-(2,3-dideoxy-2-fluoro-b-D-threo-pentofuranosyl)adenine, an acid stable purine-based reverse transcriptase inhibitor discovered by the NIH and under development by U.S. Bioscience Inc.

The term “non-nucleoside reverse transcriptase inhibitors” (“NNRTI”s) as used herein means non-nucleosides that inhibit the activity of HIV-1 reverse transcriptase. Typical suitable NNRTIs include nevirapine (BI-RG-587) available as VIRAMUNE® from Roxane Laboratories; delaviradine (BHAP, U-90152) available as RESCRIPTOR® from Pfizer; efavirenz (DMP-266) a benzoxazin-2-one disclosed in WO94/03440 and available as SUSTIVA® from Bristol-Myers Squibb Co.; PNU-142721, a furopyridine-thio-pyrimide under development by Pfizer 08807; AG-1549 (formerly Shionogi # S-1153); 5-(3,5-dichlorophenyl)-thio-4-isopropyl-1-(4-pyridyl)methyl-1H-imidazol-2-ylmethyl carbonate disclosed in WO 96/10019 and under development by Agouron Pharmaceuticals, Inc.; MKC-442 (1-(ethoxy-methyl)-5-(1-methylethyl)-6-(phenylmethyl)-(2,4(1H,3H)-pyrimidinedione) discovered by Mitsubishi Chemical Co. and under development by Triangle Pharmaceuticals; and (+)-calanolide A (NSC-675451) and B, coumarin derivatives disclosed in NIH U.S. Pat. No. 5,489,697, licensed to Med Chem Research, which is co-developing (+) calanolide A with Vita-invest as an orally administrable product.

The term “protease inhibitor” (“PI”) as used herein means inhibitors of the HIV-1 protease, an enzyme required for the proteolytic cleavage of viral polyprotein precursors (e.g., viral GAG and GAG Pol polyproteins), into the individual functional proteins found in infectious HIV-1. HIV protease inhibitors include compounds having a peptidomimetic structure, high molecular weight (7600 daltons) and substantial peptide character. Typical suitable PIs include saquinavir (Ro 31-8959) available in hard gel capsules as INVIRASE® and as soft gel capsules as FORTOVASE® from Roche Pharmaceuticals, Nutley, N.J. 07110-1199; ritonavir (ABT-538) available as NORVIR® from Abbott Laboratories; indinavir (MK-639) available as CRIXIVAN® from Merck & Co., Inc.; nelfnavir (AG-1343) available VIRACEPT® from Agouron Pharmaceuticals, Inc.; amprenavir (141W94), AGENERASE®, a non-peptide protease inhibitor under development by Vertex Pharmaceuticals, Inc. and available from Glaxo-Wellcome, under an expanded access program; lasinavir (BMS-234475) available from Bristol-Myers Squibb; DMP-450, a cyclic urea discovered by Dupont and under development by Triangle Pharmaceuticals; BMS-2322623, an azapeptide under development by Bristol-Myers Squibb as a 2nd-generation HIV-1 PI; ABT-378 under development by Abbott; and AG-1549 an orally active imidazole carbamate discovered by Shionogi and under development by Agouron Pharmaceuticals, Inc.

Other antiviral agents include hydroxyurea, ribavirin, IL-2, IL-12, pentafuside. Hydroyurea (Droxia), a ribonucleoside triphosphate reductase inhibitor, the enzyme involved in the activation of T-cells, was discovered at the NCI and is in preclinical studies, it was shown to have a synergistic effect on the activity of didanosine and has been studied with stavudine. IL-2 is disclosed in Ajinomoto EP-0142268, Takeda EP-0176299, and Chiron U.S. Pat. Nos. RE 33,653, 4,530,787, 4,569,790, 4,604,377, 4,748,234, 4,752,585, and 4,949,314, and is available under the PROLEUKIN® (aldesleukin) as a lyophilized powder for IV infusion or sc administration upon reconstitution and dilution with water; a dose of about 1 to about 20 million 1 U/day, sc is preferred; a dose of about 15 million 1 U/day, sc is more preferred. IL-12 is disclosed in WO96/25171 and is administered in a dose of about 0.5 microgram/kg/day to about 10 microgram/kg/day, sc is preferred. Pentafuside (FUZEON®) a 36-amino acid synthetic peptide, disclosed in U.S. Pat. No. 5,464,933 that acts by inhibiting fusion of HIV-1 to target membranes. Pentafuside (3-100 mg/day) is given as a continuous sc infusion or injection together with efavirenz and 2 PI's to HIV-1 positive patients refractory to a triple combination therapy; use of 100 mg/day is preferred. Ribavirin, 1-.beta.-D-ribofuranosyl-1H-1,2,4-triazole-3-carboxamide, is available from ICN Pharmaceuticals, Inc., Costa Mesa, Calif.; its manufacture and formulation are described in U.S. Pat. No. 4,211,771.

The term “viral fusion inhibitors” as used herein refers to compounds which inhibit fusion of the free virus particle and introduction of the viral RNA into a host cell independent of the molecular locus of inhibitor binding. Viral fusion inhibitors therefore include, but are not limited to T-20; CD-4 binding ligands including BMS-378806, BMS-488043; CCR5 binding ligands including SCH-351125, Sch-350634, Sch-417690 (Schering Plough), UK-4278957 (Pfizer), TAK-779 (Takeda), ONO-4128 (Ono), AK-602 (Ono, GlaxoSmithKline), compounds I-3 (Merck); CXCR4 binding ligands KRH-1636 (K. Ichiyama et al. Proc. Nat. Acad. Sci. USA 2003 100(7):4185-4190), T-22 (T. Murakami et al. J. Virol. 1999 73(9):7489-7496), T-134 (R. Arakaki et al. J. Virol. 1999 73(2):1719-1723). Viral fusion inhibitors as used herein also include peptide and protein soluble receptors, antibodies, chimeric antibodies, humanized antibodies.

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc or BOC), di-tert-butyl pyrocarbonate or boc anhydride (BOC₂O), benzyl (Bn), butyl (Bu), benzyloxycarbonyl (CBZ or Z), Chemical Abstracts Registry Number (CAS Reg. No.), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), (diphenylphosphino)ethane (dppe), (diphenylphosphino)ferrocene (dppf), 1-(3-dimethylaminopropyl)-3-ethylcarbodiimide hydrochloride (EDCl), ethyl (Et), ethyl acetate (EtOAc), ethanol (EtOH), 2-ethoxy-2H-quinoline-1-carboxylic acid ethyl ester (EEDQ), diethyl ether (Et₂O), acetic acid (HOAc), 1-N-hydroxybenzotriazole (HOBt), high pressure liquid chromatography (HPLC), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), triflate or CF₃SO₂— (Tf), trifluoro acetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), 1,1′-bis-thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### Compounds and Preparation

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2^{nd} edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

2-Benzyl-octahydro-pyrrolo[3,4-c]pyrrole (IIa) was prepared by [2,3]-dipolar cyclo addition of an imine ylide with N-benzylmaleimide as described previously (R. Colon-Cruz et al. WO 02/070523 and M. Björsne et al. WO 02/060902). Reduction of the imide, and selective debenzylation are accomplished as described therein. Pyrrolo[3,4-c]pyrrole-2(1H)-carboxylic acid, hexahydro-, 1,1-dimethylethyl ester (11b) is prepared from 11a by acylation and debenzylation (R. Colon-Cruz et al. WO 02/070523, supra).

Compounds of the present invention are prepared by step-wise elaboration of a protected octahydro-pyrrolo[3,4-c]pyrrole (11) as generally depicted in SCHEME 1. In SCHEME 1 R¹-R⁴ and X¹ are as 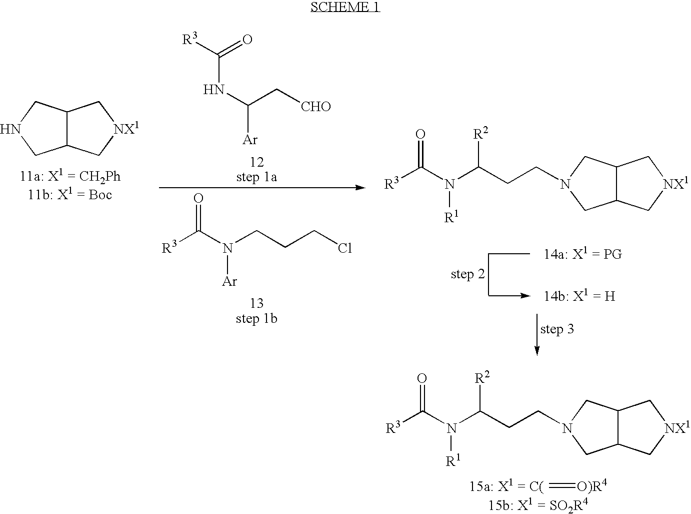
defined in SCHEME 1 and claim 1 and Ar represents optionally substituted phenyl represented by R¹ or R² in claim 1. Compounds of the present invention wherein R² is phenyl and R¹ is hydrogen are typically prepared by reductive amination of 11a or 11b with a β-amino aldehyde 12 as depicted in step 1a to afford 14a wherein R¹ is hydrogen and R² is aryl. Compounds of the present invention wherein R¹ is phenyl and R² is hydrogen are typically prepared by alkylation of 11a or 11b with an alkyl halide 13 as depicted in step 1b to afford 14a wherein R¹ is aryl and R² is hydrogen. After the first nitrogen substitutent is introduced the protecting group is removed to afford 14b and the second nitrogen is acylated to afford 15a or sulfonylated to afford 15b. One skilled in the art will appreciate that the sequence of these steps can be reversed such that the acyl/sulfonylation is carried out first on 11a or 11b and the reductive amination/alkylation is carried out after removal of the nitrogen protecting group.

Reductive amination is preferably carried out carried out by combining an amine and carbonyl compound in the presence of a complex metal hydride such as sodium borohydride, lithium borohydride, sodium cyanoborohydride, zinc borohydride, sodium triacetoxyborohydride or borane/pyridine conveniently at a pH of 1-7 or with hydrogen in the presence of a hydrogenation catalyst, e.g. in the presence of palladium/charcoal, at a hydrogen pressure of 1 to 5 bar, preferably at temperatures between 20° C. and the boiling temperature of the solvent used. Optionally a dehydrating agent, such as molecular sieves or Ti(IV)(O-i-Pr)₄, is added to facilitate formation of the intermediate imine at ambient temperature. It may also be advantageous to protect potentially reactive groups during the reaction with conventional protecting groups which are cleaved again by conventional methods after the reaction. Reductive amination procedures have been reviewed: R. M. Hutchings and M. K. Hutchings Reduction of C═N to CHNH by Metal Hydrides in Comprehensive Organic Synthesis col. 8, I. Fleming (Ed) Pergamon, Oxford 1991 pp. 47-54.

The amine alkylation is accomplished by treating the amine or a metal salt of the amine (i.e. a deprotonated form) with a compound RZ¹ wherein Z¹ is a leaving group such as halo, C₁₋₄ alkanesulphonyloxy, benzenesulphonyloxy or p-toluenesulphonyloxy. In the example depicted in SCHEME 1 RZ¹ is 13 and Z¹ is chloride. The reaction is optionally carried out in the presence of a base and/or a phase transfer catalyst. Commonly used bases include, but are not limited to, TEA, DIPEA or DBU; or an inorganic base such as Na₂CO₃, NaHCO₃, K₂CO₃ or Cs₂CO₃. Commonly used solvents include, but are not limited to acetonitrile, DMF, DMSO, 1,4-dioxane, THF or toluene. The reaction is conveniently run by in the presence of NaI which forms a more reactive intermediate alkyl iodide (i.e., Z¹ is iodide).

The acylation is conveniently carried out with a corresponding acyl halide or acid anhydride in a solvent such as DCM, chloroform, carbon tetrachloride, ether, THF, dioxane, benzene, toluene, MeCN, DMF, aqueous sodium hydroxide solution or sulfolane optionally in the presence of an inorganic or organic base at temperatures between −20 and 200° C., but preferably at temperatures between −10 and 100° C. Typical organic bases include tertiary amines include but are not limited to TEA, pyridine. Typical inorganic bases include but are not limited to K₂CO₃ and NaHCO₃.

The acylation may however also be carried out with the free acid optionally in the presence of an acid-activating agent or a dehydrating agent, e.g. isobutyl chloroformate, carbodiimides such as EDCl or DCC optionally in the presence of an additive such as HOBt or N-hydroxysuccinimide, O-(benzotriazol-1-yl)-N,N,N′,N′-tetramethyl-uronium tetrafluoroborate (TBTU) in the presence of a base such as DIPEA or NMM, N,N′-carbonyldiimidazole, N,N′-thionyldiimidazole or triphenylphosphine/carbon tetrachloride, at temperatures between −20 and 200° C., but preferably at temperatures between −10 and 100° C.

A sulfonylation maybe conveniently carried out by treating the amine with a sulfonyl chloride in a solvent such as DCM, chloroform, carbon tetrachloride, ether, THF, dioxane, benzene, toluene, MeCN, DMF, aqueous sodium hydroxide solution or sulfolane in the presence of an organic base such as amines which include but are not limited to TEA, pyridine at temperatures between −10 and 120° C.

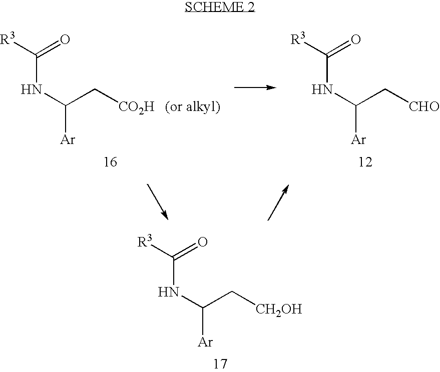

The β-acylamino aldehyde 12 can be prepared by direct reduction of a β-acylamino acid 16 or ester with a hydride reducing agent such as DIBAL-H or 16 can be reduced to the corresponding alcohol and re-oxidized to the aldehyde with a SO₃-pyridine and TEA or alternative oxidizing agent. The R³ moiety can either be the moiety present in the final compound or alternatively R³C(═O) can be a protecting group, e.g., R³═O-tert-Bu (BOC), which can be removed to liberate a primary amine at an advantageous time which can be acylated or sulfonylated as desired.

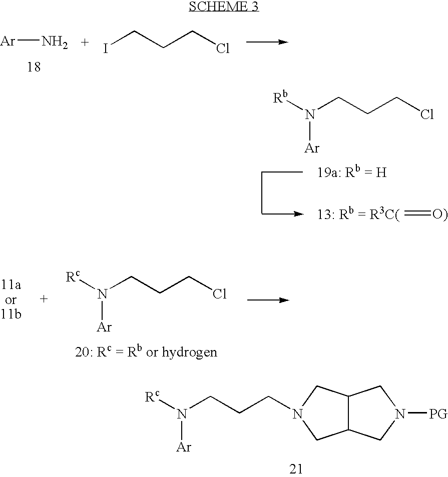

The 3-chloro-propyl-N-aryl-amine 19a is prepared by alkylation of an optionally substituted aniline 18 with 3-iodo-1-chloro-propane. Alkylation of 11a or 11b can be accomplished with the secondary amine

19a which is subsequently acylated; or, alternatively the secondary amine can be acylated to afford 13 wherein R³ is as defined in claim 1 or R³C(═O) is a protecting group which is removed to allow acylation of the secondary amine. Removal of the protecting group of 11 and acylation or sulfonylation as described above affords 15a or 15b respectively wherein R¹ is aryl and R² is hydrogen.

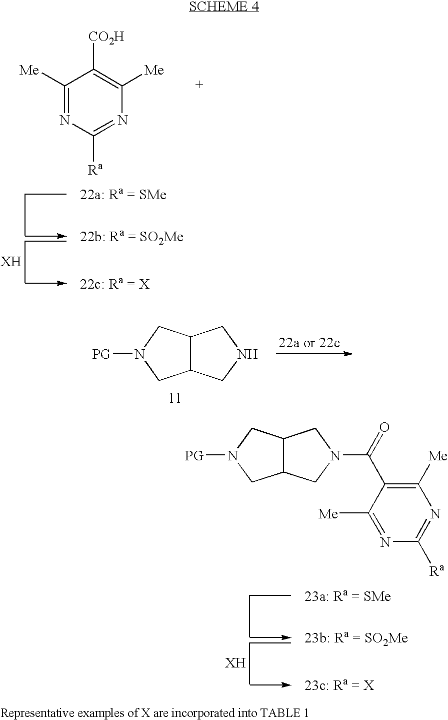

Compounds (I-1 to I-38) tabulated in TABLE 1 comprise 2-oxy- or 2-amino substituted pyrimidines. Introduction of the 2-oxy or 2-amino-substitutent is readily accomplished by displacement of methane-sulfinic acid from a 2-methanesulfonyl-pyrimidine by an amine or alcohol. As depicted in SCHEME 4, the displacement can be carried out before (22b to 22c) or after (23b to 23c) the pyrimidine is linked to the octahydro-pyrrolo[3,4-c]pyrrole scaffold.

Examples of representative compounds encompassed by the present invention and within the scope of the invention are provided in the following Tables. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE 1

| | 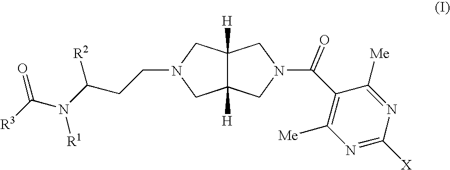 | | | | | |
|---|---|---|---|---|---|---|
| Comp | | | | | ms | |
| No. | R¹ | R² | R³ | X | M + H | salt |
| I-1 | H | Ph | Cyp | OCH₂C(═O)NHMe | 563 | |
| I-2 | H | Ph | Cyp | OCH₂C(═O)NH₂ | 549 | |
| I-3 | H | Ph | Cyp | OCH₂CO₂CH₂Ph | 640 | |
| I-4 | H | Ph | Cyp | OCHMeCO₂Me | 578 | |
| I-5 | H | Ph | Cyp | NHCH₂CO₂H | 549 | TFA |
| I-6 | H | Ph | Cyp | OCHMeCO₂H | 564 | TFA |
| I-7 | H | Ph | Cyp | O-p-C₆H₄—CO₂CH₂Ph | 702 | |
| I-8 | H | Ph | Cyp | O-p-C₆H₄—CO₂H | 612 | TFA |
| I-9 | H | Ph | Cyp | O-m-C₆H₄—CO₂Et | 640 | |
| I-10 | H | Ph | Cyp | O-m-C₆H₄—CO₂H | 612 | HCl |
| I-11 | H | Ph | Cyp | (R)-OCH₂CHMeCO₂Me | 592 | TFA |
| I-12 | H | Ph | Cyp | (R)-OCH(Ph)CO₂Me | 640 | |
| I-13 | H | Ph | Cyp | (S)-OCH(Me)CO₂Me | 578 | |
| I-14 | H | Ph | Cyp | O-CH(Et)CO₂H | 578 | |
| I-15 | H | Ph | Cyp | (S)-OCH(Me)CO₂H | 562¹ | |
| I-16 | H | Ph | Cyp | (S)-OCH(Ph)CO₂Me | 640 | |
| I-17 | H | Ph | Cyp | (S)-OCH(Ph)CO₂H | 626 | |
| I-18 | H | Ph | Cyp | (R)-OCH(Ph)CO₂H | 626 | |
| I-19 | H | Ph | Cyp | OMe | 506 | |
| I-20 | H | Ph | Cyp | (R)-OCH(Me)CO₂H | 562¹ | |
| I-21 | H | Ph | Cyp | O-CH(Et)CO₂Et | 606 | |
| I-22 | H | Ph | Cyp | (R)-OCH(Me)CO₂Et | 592 | |
| I-23 | H | Ph | Cyp | 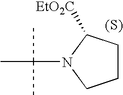 | 617 | |
| I-24 | H | Ph | Cyp | 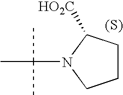 | 589 | |
| I-25 | Cl-Me-Ph | H | Di-F-CyH | 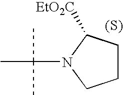 | 715 | |
| I-26 | H | Ph | Cyp | OCMe₂CO₂Me | 592 | |
| I-27 | H | Ph | Cyp | (S)-NHCH(i-Pr)CO₂Et | 619 | |
| I-28 | Cl-Me-Ph | H | Di-F-CyH | (S)-NHCH(i-Pr)CO₂Et | 717 | |
| I-29 | Cl-Me-Ph | H | Di-F-CyH | 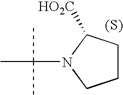 | 687 | |
| I-30 | Cl-Me-Ph | H | Di-F-CyH | (S)-NHCH(i-Pr)CO₂H | 688² | |
| I-31 | H | Ph | Cyp | (S)-NHCH(i-Pr)CO₂H | 591 | |
| I-32 | H | Ph | Cyp | OCMe₂CO₂H | 578 | |
| I-33 | H | Ph | Cyp | (S)-NHCH(Me)CO₂Et | 591 | |
| I-34 | Cl-Me-Ph | H | Di-F-CyH | (S)-NHCH(Me)CO₂Et | 689 | |
| I-35 | H | Ph | Cyp | (S)-NHCH(Me)CO₂H | 563 | |
| I-36 | Cl-Me-Ph | H | Di-F-CyH | (S)-NHCH(Me)CO₂H | 661 | |
| I-37 | Cl-Me-Ph | H | Di-F-CyH | —NMeCH₂CO₂Et | 689 | |
| I-38 | Cl-Me-Ph | H | Di-F-CyH | —NMeCH₂CO₂H | 661 | |
| I-39 | H | Ph | Cyp | 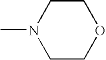 | 561 | |
| I-40 | H | Ph | Cyp | 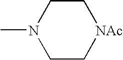 | 602 | |
| I-41 | H | Ph | Cyp | 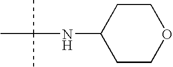 | 575 | |
| I-42 | H | Ph | Cyp | —NHSO₂Me | 569 | |
| I-43 | H | Ph | Cyp | —N(Me)SO₂Me | 583 | |

| I-44 | 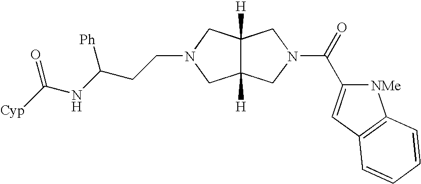 | | | | 499 |
|---|---|---|---|---|---|
| I-45 | 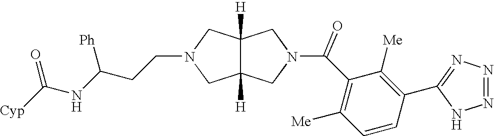 | | | | 542 |
| I-46 | H | Ph | Cyp | 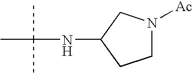 | 602 |
| I-47 | H | Ph | Cyp | 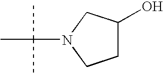 | 561 |
| I-48 | H | 3-F-Ph | 3THF | c-C₃H₅ | 536 |
| I-49 | H | Ph | Di-F-CyB | 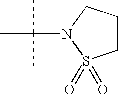 | 617 |
| I-50 | H | Ph | Di-F-CyB | —NHCH₂CH(Me)OH | 570 |
| I-51 | H | Ph | Di-F-CyB | 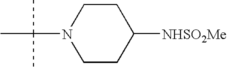 | 673 |

| | | |
|---|---|---|
| I-52 | 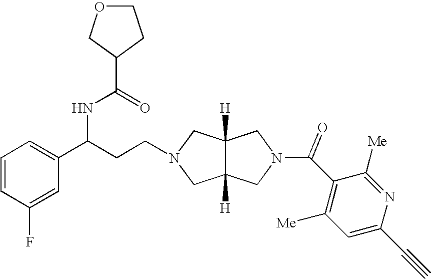 | 519 |

¹[M − H]
²[M] electron impact
Cyp = cyclopentyl
Di-F-CyH = 4,4-difluorocyclohexyl
Cl-Me-Ph = 3-Cl-4-Me-C₆H₃
Di-F-CyB = 4,4-difluorocyclobutyl
3-THF = tetrahydrofuran-3-yl

The 3,5-dimethyl-1H-pyrazole-4-carboxy derivatives are tabulated in TABLE 2. Cyclo alkyl substituted pyrazoles were prepared by cyclo-condensation of 2-acetyl-3-oxo-butyric acid ethyl ester (26) and a cycloalkylhydrazine 25 to afford 27 (R=cycloalkyl). Optionally substituted N-pyrazines and N-pyridazines can be prepared by displace of a chloro substitutent on the heteroaryl ring with 3,5-dimethyl-1H-pyrazole-4-carboxylic acid methyl ester (27, R=H) to afford 28. (SCHEME 5) Displacement of leaving groups on other electrophilic carbon atoms, e.g., reaction with bromo acetic acid derivatives to afford 29, can also be readily accomplished. Hydrolysis of the ester and condensation with an appropriate octahydro-pyrrolo[3,4-c]pyrrole affords compounds exemplified in TABLE 2. In general the preformed pyrazole is prepared prior to incorporation onto the scaffold; however the sequence of reactions has sufficient flexibility to permit alternative routes to be adopted.

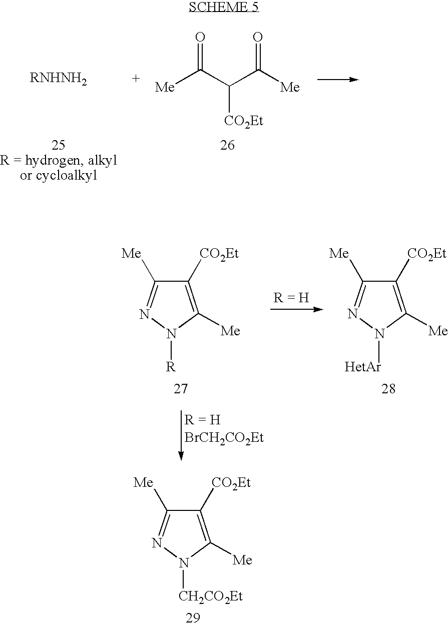

TABLE 2

| | 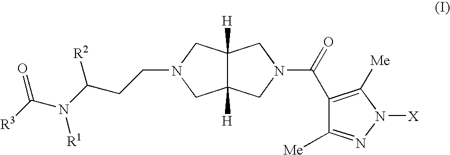 | | | | |
|---|---|---|---|---|---|
| | R¹ | R² | R³ | X | ms |
| II-1 | H | Ph | Cyp | pyridazin-3-yl | 542 |
| II-2 | H | Ph | i-Pr | pyridazin-3-yl | 516 |
| II-3 | H | 3-F—C₆H₄ | 3-THF | 5-CF₃-pyridin- | 629 |
| | | | | 2-yl | |
| II-4 | H | 3-F—C₆H₄ | Me | 5-CF₃-pyridin- | 573 |
| | | | | 2-yl | |
| II-5 | H | 3-F—C₆H₄ | Cyp | pyrimidin-5-yl | 560 |
| II-6 | H | 3-F—C₆H₄ | MeO | 5-CF₃-pyridin- | 589 |
| | | | | 2-yl | |
| II-7 | H | 3-F—C₆H₄ | Me | cyclohexyl | 510 |
| II-8 | H | 3-F—C₆H₄ | 3-THF | cyctohexyl | 566 |
| II-9 | H | 3-F—C₆H₄ | Cyp | cyclohexyl | 564 |
| II-10 | H | 3-F—C₆H₄ | Cyp | cyclobutyl | 536 |
| II-11 | H | 3-F—C₆H₄ | i-Pr | cyclobutyl | 510 |
| II-12 | H | 3-F—C₆H₄ | 3-THF | cyclobutyl | 538 |
| II-13 | H | 3-F—C₆H₄ | Cyp | 6-CF₃- | 628 |
| | | | | pyridazin-3-yl | |
| II-14 | H | 3-F—C₆H₄ | Me | 6-CF₃- | 574 |
| | | | | pyridazin-3-yl | |
| II-15 | H | 3-F—C₆H₄ | i-Pr | 6-CF₃- | 602 |
| | | | | pyridazin-3-yl | |
| II-16 | H | 3-F—C₆H₄ | 3-THF | 6-CF₃- | 630 |
| | | | | pyridazin-3-yl | |
| II-17 | H | 3-F—C₆H₄ | 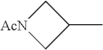 | 6-CF₃- pyridazin-3-yl | 657 |
| II-18¹ | Cl- Me- Ph | H | 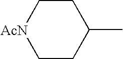 | CH₂CO₂H | — |
| II-19 | H | Ph | Cyp | CH₂CO₂H | 522 |
| II-20 | H | 3-F—C₆H₄ | Me | 6-CH₃- | 520 |
| | | | | pyridazin-3-yl | |
| II-21 | H | 3-F—C₆H₄ | 3-THF | 6-CH₃- | 576 |
| | | | | pyridazin-3-yl | |
| II-22 | H | 3-F—C₆H₄ | i-Pr | 6-CH₃- | 548 |
| | | | | pyridazin-3-yl | |
| II-23 | H | 3-F—C₆H₄ | Cyp | 6-CH₃- | 574 |
| | | | | pyridazin-3-yl | |
| II-24 | H | 3-F—C₆H₄ | 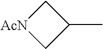 | 6-CH₃- pyridazin-3-yl | 603 |
| II-25 | H | 3-F—C₆H₄ | 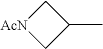 | pyrazin-3-yl | 589 |
| II-26 | H | 3-F—C₆H₄ | Me | pyrazin-3-yl | 506 |
| II-27 | H | 3-F—C₆H₄ | i-Pr | pyrazin-3-yl | 534 |
| II-28 | H | 3-F—C₆H₄ | Cyp | pyrazin-3-yl | 560 |
| II-29 | H | 3-F—C₆H₄ | 3-THF | pyrazin-3-yl | 562 |

¹TFA salt
Cyp = cyclopentyl
3-THF = tetrahydrofuran-3-yl
Cl-Me-Ph = 3-Cl-4-Me-C₆H₃

Pyridones (34) and 2-(carboxyalkoxy)pyridines (35) tabulated in TABLE 3 were prepared by acylation of 11 by 31 or 32b. N-alkyl compounds 32b were prepared by alkylation of 31 and subsequent hydrolysis of the resulting ester. The scheme is depicted with a protecting group on N^{b}, however one skilled in the art would recognize that N^{b} could also be a substituted with a moiety present in the final compound (e.g., Ar—N(BOC)(CH₂)₃—). Alkylation of 31 is readily accomplished by a base (e.g., Cs₂CO₃ or NaH) and an alkylating agent in a non-protic solvent. When R^{b} is hydrogen, alkylation with an haloester affords a mixture N- and O-alkyl compounds 34 and 35 which were separable by chromatography and which could subsequently be hydrolyzed to the corresponding carboxyalkyl substitutent (e.g., 34 or 35, R^{b} is CH₂CO₂H. Specific examples of the sequence are found in the following examples

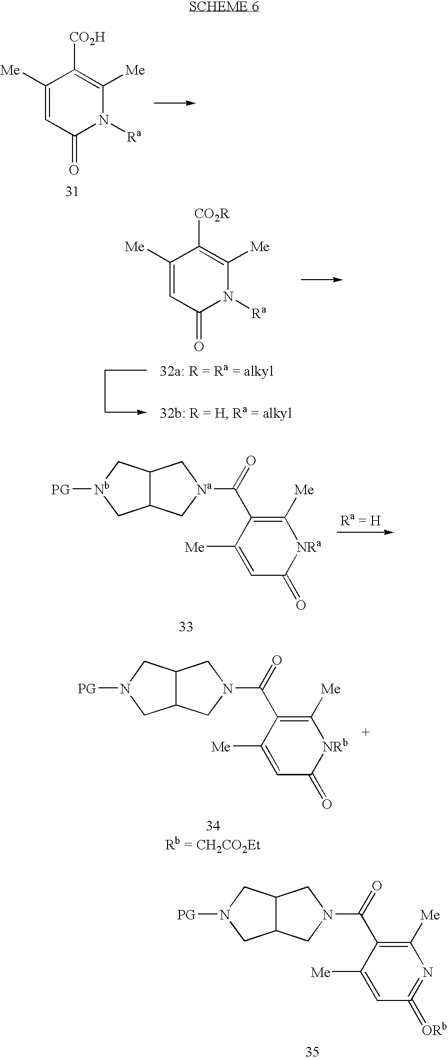

TABLE 3

| | |
|---|---|
| | (I) |
| 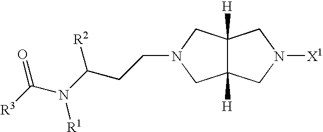 | |
| 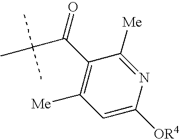 | (A) |
| 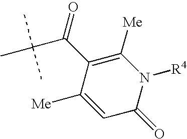 | (B) |

| | R¹ | R² | R³ | X¹ | R⁴ | ms | mp | |
|---|---|---|---|---|---|---|---|---|
| III-1 | Cl-Me-Ph | H | Ac-Pip | B | CH₂CO₂Et | 682 | 88.2-89.1 | |
| III-2 | Cl-Me-Ph | H | Ac-Pip | B | CH₂CO₂H | 654 | | TFA |
| III-3 | H | Ph | Cyp- | B | H | 491 | | |
| III-4 | Cl-Me-Ph | H | Ac-Pip | A | CH₂CO₂H | 654 | | TFA |
| III-5 | Cl-Me-Ph | H | Ac-Pip | B | H | 596 | | TFA |
| III-6 | Cl-Me-Ph | H | Ac-Pip | A | CHMeCO₂Et | 696 | | |
| III-7 | Cl-Me-Ph | H | Ac-Pip | A | CHMeCO₂H | 668 | | |
| III-8 | Cl-Me-Ph | H | di-F-CyH | B | H | 589 | | |
| III-9 | Cl-Me-Ph | H | di-F-CyH | B | CH₂CO₂Et | 675 | | |
| III-10 | Cl-Me-Ph | H | di-F-CyH | B | CH₂CO₂H | 647 | | |
| III-11 | H | 3-F—C₆H₄ | Cyp- | B | H | 509 | | |
| III-12 | H | 3-F—C₆H₄ | di-F-CyH | B | H | 559 | | |
| III-13 | H | Ph | Cyp- | B | Me | 505 | | |
| III-14 | H | 3-F—C₆H₄ | 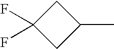 | B | H | 531 | | |
| III-15 | H | Ph | di-F-CyH | B | Me | 555 | | |
| III-16 | H | Ph | 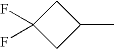 | B | Me | 527 | | |
| III-17 | H | Ph | 3-THF | B | Me | 507 | | |
| III-18 | H | Ph | 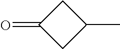 | B | Me | 505 | | |
| III-19 | H | Ph | 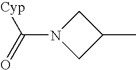 | B | Me | 588 | | |
| III-20 | 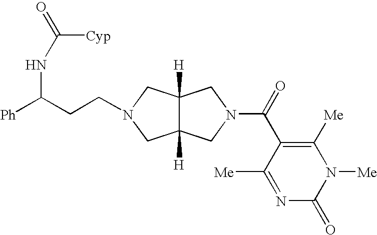 | | | | | 506 | | |
| III-21 | 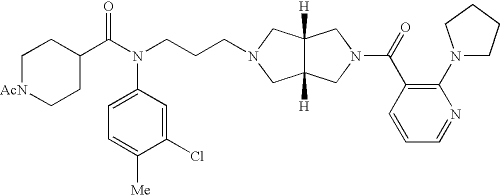 | | | | | 622.2 | TFA | |
| III-22 | 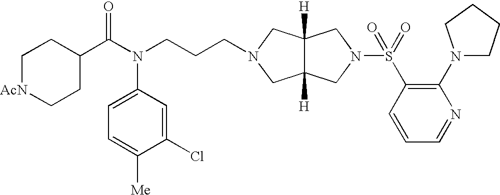 | | | | | 657.4 | TFA | |
| III-23 | H | Ph | di-F-CyB | B | —CH₂CF₃ | 595 | | |
| III-24 | H | Ph | Cyp | B | CH₂C(O)Me | 547 | | |
| III-25 | H | Ph | Cyp | B | CH₂CHOHMe | 549 | | |
| III-26 | H | Ph | di-F-CyB | A | CH₂C(O)Me | 569 | | |
| III-27 | H | Ph | 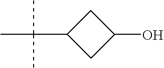 | A | CH₂CH(OH)Me | 550 | | |

| | | |
|---|---|---|
| III-28 | 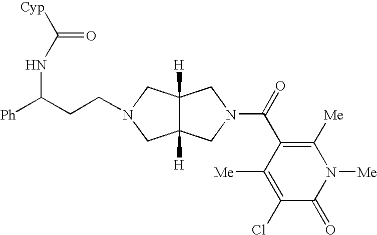 | 526 |

Cyp =cyclopentyl
Ac-Pip = N-acetyl-piperidin-4-yl
di-F-CyH = 4,4-difluorocyclohexyl
3-THF = 3-tetrahydro-furan-3-yl
di-F-CyB = 3,3-difluorocyclobutyl

### Dosage and Administration

The compounds of the present invention may be formulated in a wide variety of oral administration dosage forms and carriers. Oral administration can be in the form of tablets, coated tablets, dragées, hard and soft gelatine capsules, solutions, emulsions, syrups, or suspensions. Compounds of the present invention are efficacious when administered by other routes of administration including continuous (intravenous drip) topical parenteral, intramuscular, intravenous and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylactic acid, tertiary butylactic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It should be understood that all references to pharmaceutically acceptable salts include solvent addition forms (solvates) or crystal forms (polymorphs) as defined herein, of the same acid addition salt.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, color ants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable color ants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyacetic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 100 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

In embodiments of the invention, the active compound or a salt can be administered in combination with another antiviral agent, such as a nucleoside reverse transcriptase inhibitor, another non-nucleoside reverse transcriptase inhibitor or HIV protease inhibitor. When the active compound or its derivative or salt are administered in combination with another antiviral agent the activity may be increased over the parent compound. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

It will be understood that references herein to treatment extend to prophylaxis as well as to the treatment of existing conditions, and that the treatment of animals includes the treatment of humans as well as other primates. Furthermore, treatment of a HIV-1 infection, as used herein, also includes treatment or prophylaxis of a disease or a condition associated with or mediated by HIV-1 infection, or the clinical symptoms thereof.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

### EXAMPLE 1

Cyclopentanecarboxylic acid {(S)-3-[5-(2-carbamoylmethoxy-4,6-dimethyl-pyrimidine-5-carbonyl)-hexahydro-pyrrolo[3,4-c]pyrrol-2-yl]-1-phenyl-propyl}-amide (I-2) and (5-{5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yloxy)-acetic Acid Benzyl Ester (I-3)

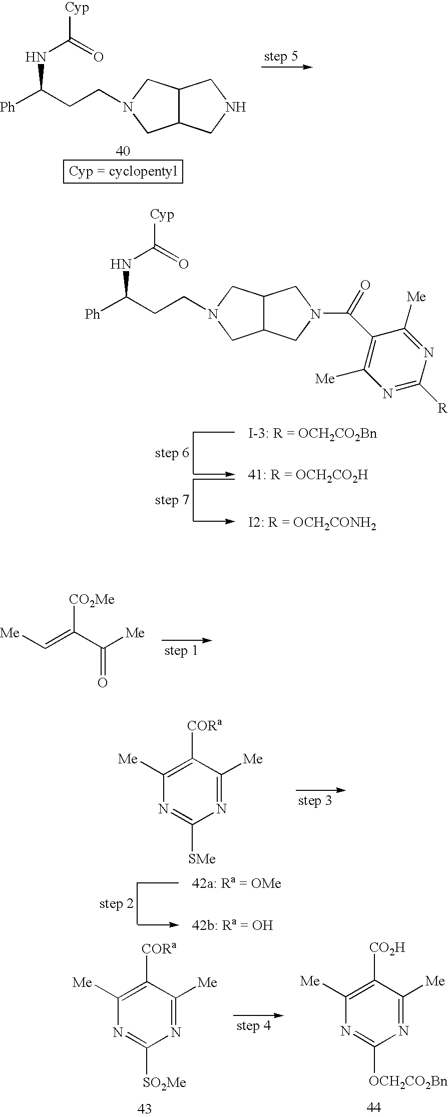

step 1—To a mixture of 2-acetyl-3-methoxy-but-2-enoic acid methyl ester (WO2005/007608) (34.4 g, 0.200 mol) and 2-methyl-2-thiopseudourea sulfate (33.4 g, 0.120 mol) in a mixture of acetone/MeOH (1.4/1, 240 mL) cooled in a ice-water bath (<5° C.) was added dropwise a slurry of potassium tert-butoxide (27.1 g, 0.24 mol) in THF (100 mL) while maintaining the temperature below 5° C. When the addition was complete the ice bath was removed and the reaction was stirred at RT. After 2 h the mixture was neutralized (pH=7) by addition of con HCl (ca. 5 mL) and filtered. The filter cake washed with EtOAc, the filtrate was evaporated and the residue was partitioned between water (90 mL) and tert-BuOMe (140 mL). The layers were separated and the aqueous phase was extracted with tert-BuOMe (100 mL), then twice with a mixture of EtOAc/tert-BuOMe (1/1, 100 mL). The organic layers were combined and dried (MgSO₄), filtered and concentrated to give a brown oil that solidified on standing. This material was purified via SiO₂ chromatography eluting with hexane/EtOAc to afford 32.1 g (76%) of 42a as white solid.

step 2—To a suspension of 42a (31.9 g, 0.150 mol) in a 1:1 mixture of water/MeOH (54 mL) was added a solution of NaOH (6.33 g, 0.158 mol) in water (20 mL). The mixture was stirred at 50° C. overnight, the temperature was then raised to 60° C. and the reaction was stirred for another 5 h. The reaction was then cooled with and ice water bath (<5° C.) and con HCl (22.5 mL) was added. During this process the mixture became very thick and unstirrable. Water was added (200 mL) and a mechanical stirrer was used. The reaction was stirred for 1 h at <5° C. then filtered and the filter cake washed twice with water (50 mL). The solid was dried in vacuo to afford 28.2 g (96%) of 42b.

step 3—To a solution of 42b (1 g, 5.1 mmol) in MeOH (30 mL) cooled to 0° C. was added a solution of oxone (6.9 g, 11 mmol) in water (30 mL). The reaction was stirred at RT overnight then cooled at 0° C and diluted with water. The mixture was extracted with EtOAc and DCM. The combined organic layers were dried (MgSO₄), filtered and concentrated in vacuo to afford 0.950 g of 43 which used without further purification.

step 4—A mixture of 43 (400 mg, 1.74 mmol), hydroxy-acetic acid benzyl ester (0.394 mL, 2.78 mmol) and Cs₂CO₃ (1.19 g, 3.65 mmol) and DMF (95 mL) was heated to 70° C. overnight. The same amount of hydroxy-acetic acid benzyl ester and Cs₂CO₃ was then added and heating at 73° C. was continued for 62 hours. The reaction was cooled at RT and water was added. The mixture was acidified by addition of HCl (1M) and extracted with EtOAc. The combined organic layers were dried (MgSO₄), filtered and concentrated. The residue was purified via SiO₂ chromatography eluting with DCM/MeOH/NH₄OH (60/10/1) to afford 0.213 g of 44.

Cyclopentanecarboxylic acid ((S)-3-oxo-1-phenyl-propyl)-amide (40) was prepared by the procedures used to prepare 48b in steps 3-6 of example 2 except (S)-3-(cyclopentanecarbonyl-amino)-3-phenyl-propionic acid was used in place of 47a in step 3.

step 5—To a suspension of 40 (209 mg, 0.612 mmol) in DCM (3 mL) was added sequentially 44 (213 mg, 0.674 mmol), HOBt (124 mg, 0.516 mmol), EDCl (164 mg, 0.857 mmol) and TEA (171 μL, 1.22 mmol). The reaction was stirred at RT for 22 h and quenched by addition of water. The mixture was extracted with DCM, the combined organic layers were dried (MgSO₄), filtered and concentrated. The residue was purified via preparative TLC on SiO₂ and developed with DCM/MeOH/NH₄OH (60/10/1) to afford 0.310 g of I-3

step 6—A mixture of I-3 (300 mg, 0.47 mmol) and Pd/C (10%, 61 mg) and EtOH (15 mL) was stirred under H₂ atmosphere (balloon pressure) at RT for 4 h. The reaction mixture was filtered thorough a CELITE® pad and rinsed with EtOH. The filtrate was evaporated in vacuo to afford 0.230 g of 41 which was used in the next step without additional purification.

step 7—To a mixture of 41 (30 mg, 0.0546 mmol) and ammonia (0.5M solution in 1,4-dioxane, 99 μL, 0.0496 mmol) in DCM (0.25 mL) was added HOBt (10 mg, 0.0742 mmol) followed by EDCl (13 mg, 0.0692 mmol) and TEA (14 μL, 0.099 mmol). The reaction was stirred at RT overnight and then the same amount of ammonia, EDCl, HOBt and TEA was added until completion of the reaction. The mixture was purified via preparative SiO₂ TLC and developed with DCM/MeOH/NH₄OH (60/10/1) to afford 0.017 g of I-2.

Cyclopentanecarboxylic acid {(S)-3-[5-(4,6-dimethyl-2-methylcarbamoylmethoxy-pyrimidine-5-carbonyl)-hexahydro-pyrrolo[3,4-c]pyrrol-2-yl]-1-phenyl-propyl}-amide (I-1) was prepared analogously except in step 7 the solution of ammonia in dioxane was replaced by a solution of N-methylamine in dioxane.

### EXAMPLE 2

### Cyclopentanecarboxylic Acid [(S)-3-[5-(3,5-dimethyl-1-pyrimidin-5-yl-1H-pyrazole-4-carbonyl)-hexahydro-pyrrolo[3,4-c]pyrrol-2-yl]-1-(3-fluoro-phenyl)-propyl]-amide (II-5)

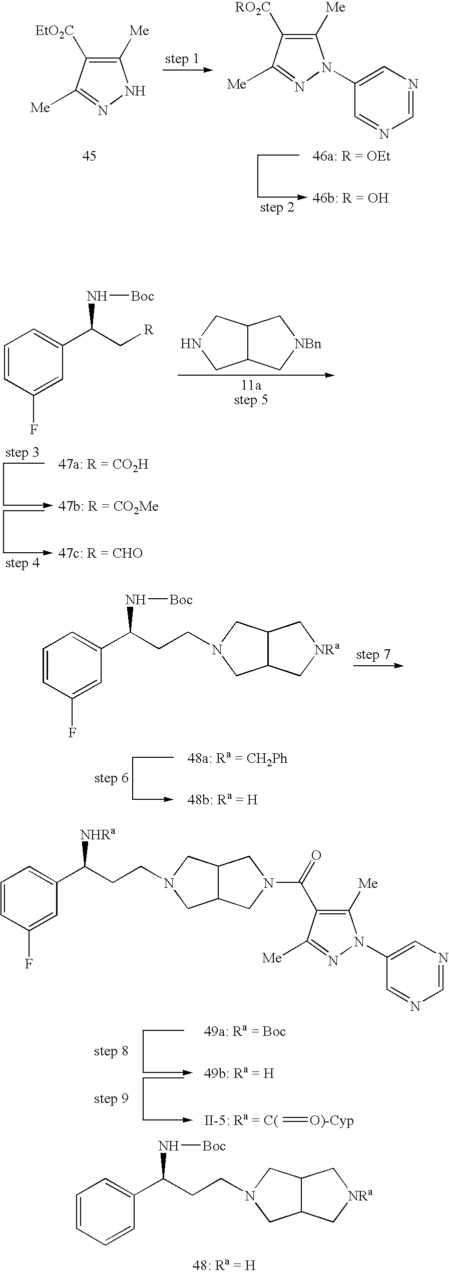 step 1—N,N′-Dimethylethylenediamine (90 μL, 0.832 mmol) was added to a mixture of 3,5-dimethyl-1H-pyrazole-4-carboxylic acid ethyl ester (45, 1.4 g, 8.324 mmol), 5-bromopyrimidine (1.32 g, 8.303 mmol), CuI (0.16 g, 0.84 mmol) and K₂CO₃ (2.3 g, 16.64 mmol) in 1,4-dioxane (8 mL) that was maintained under an Ar atmosphere. The resulting mixture was stirred at 110° C. under Ar for 16 h. The reaction mixture was cooled to RT, diluted with DCM (50 mL) and filtered through a CELITE® and SiO₂ pad. The filter cake was rinsed with EtOAc and the filtrate was evaporated in vacuo. The residue was purified via SiO₂ chromatography eluting with hexane/EtOAc to afford the 0.150 g (7%) of 46a.
step 2—A solution of KOH (77 mg, 1.38 mmol) in water (0.5 mL, plus 0.25 mL to rinse) was added to a solution of 46a (170 mg, 0.69 mmol) in EtOH (3 mL). The resulting mixture was stirred at 40° C. for 24 h, cooled to RT and evaporated in vacuo. The residue was partitioned between EtOAc and water and the resulting aqueous layer was separated and extracted with EtOAc. The aqueous layer was acidified to pH 4 with 3M HCl. The precipitate was filtered and rinsed with water to afford 0.086 g (57%) of 46b which was used for the next step without additional purification.
step 3—To a solution of 47a (10.0 g, 35.3 mmol) in MeOH (100 mL) under N₂ atmosphere cooled in an ice-water bath was added DCC (8.74 g, 42.4 mmol) followed by DMAP (431 mg, 3.52 mmol). The mixture was stirred at RT overnight, filtered and the filtrate was evaporated. The residue was purified via SiO₂ chromatography eluting with hexane/EtOAc to afford 9.71 g (93%) of 47b.
step 4—To a solution of 47b (9.71 g, 32.6 mmol) in DCM (300 mL) maintained under N₂ atmosphere and cooled at −78° C. was added a solution of DIBAL-H (1M in DCM, 65.3 mL) at a rate that maintained the temperature at less than 70° C. The reaction was then stirred 2 h at −78⁰ C then quenched by addition of MeOH (85 mL) at a rate that maintained the temperature at less than <70° C. The mixture was warmed to RT, washed sequentially with 2M HCl and brine, dried (Na₂SO₄), filtered and evaporated to afford 9.32 g of 47c as viscous liquid.
step 5—A mixture of 47c (93.4 mg, 0.349 mmol), 2-benzyl-octahydro-pyrrolo[3,4-c]pyrrole (11a, 71.7 mg, 0.354 mmol), NaBH(OAc)₃ (93.3 mg, 0.440 mmol) and HOAc (52 □L, 0.908 mmol) in DCM (4 mL) was stirred overnight at RT. The reaction was quenched by addition of 10% aqueous K₂CO₃ (4 mL) and the resulting mixture stirred for 30 min. The resulting mixture was partitioned between water and DCM. The organic layer was separated and the aqueous layer was extracted with DCM. The combined organic extracts were dried (MgSO₄), filtered and concentrated in vacuo. The crude product was purified via SiO₂ chromatography eluting with DCM/MeOH to afford 0.108 g (68%) of 48a as viscous oil: M+H=454.
step 6—A mixture of 48a (108 mg, 0.238 mmol), ammonium acetate (154 mg, 2.44 mmol) and Pd(OH)₂/C (20%, 63 mg) in EtOH was heated at reflux for 5 h and then stored in the freezer overnight. The catalyst was filtered through a CELITE® pad and the filtrate was evaporated an adsorbed onto SiO₂. The resulting SiO₂ applied to the top of a SiO₂ column and eluted with DCM/MeOH to afford 0.115 g of impure 48b as oil: M+H=364.
step 7—DIPEA (80 μL, 0.454 mol) was added to a solution of 48b (110 mg, 0.303 mmol), 46b (73 mg, 0.333 mmol), EDCl (70 mg, 0.363 mmol) and HOBt (56 mg, 0.363 mmol) in DCM (3 mL). The resulting mixture was stirred at RT for 24 h then partitioned between water and DCM. The layers were separated and the aqueous layer was extracted twice with DCM. The combined organic layers were dried (Na₂SO₄), filtered and evaporated. The residue was purified via SiO₂ chromatography which afforded a partially purified 49a which was used in the following step.
step 8—TFA (1 mL) was added to a solution of 49a from step 3 in DCM (1 mL). The reaction was stirred at room temperature overnight, evaporated and the residue was purified via SiO₂ chromatography and eluted with DCM/MeOH/NH₄OH (60/10/1) to afford 0.054 g of 49b.
step 9—Cyclopentanecarbonyl chloride (20 μL, 0.140 mmol) was added to a solution of 49b (54 mg, 0.116 mmol) in a 3/1 mixture DCM/pyridine (1 mL). The mixture was stirred over the weekend; quenched by addition of MeOH (1 mL) and evaporated. The residue was purified via SiO₂ chromatography eluting with DCM/MeOH/NH₄OH (60/10/1) to afford 0.050 g of II-5.

### EXAMPLE 3

### Cyclopentanecarboxylic Acid {(S)-3-[(5-(3,5-dimethyl-1-pyridazin-3-yl-1H-pyrazole-4-carbonyl)-hexahydro-pyrrolo[3,4-c]pyrrol-2-yl]-1-phenyl-propyl}-amide (II-1)

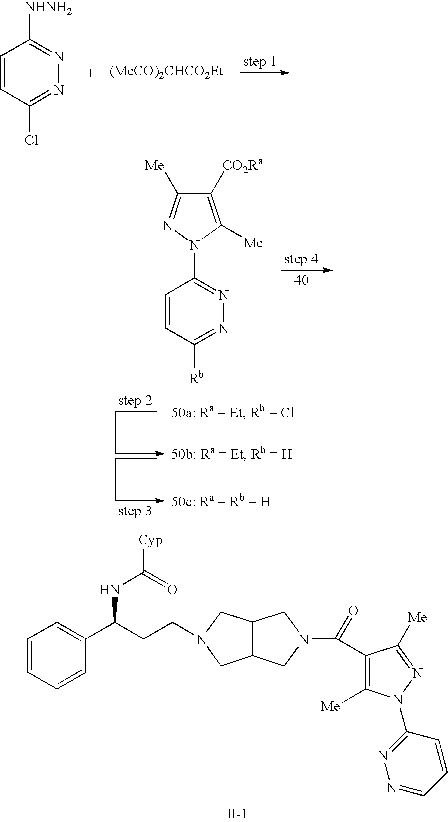 step 1—Ethyl diacetoacetate (2 mL, 12.8 mmol) was added at RT to a mixture of 3-chloro-6-hydrazinopyridazine (1.5 g, 10.4 mmol) and HOAc (1 mL) in MeOH (30 mL). The resulting mixture was stirred at RT for 1 h. The resulting precipitate formed was filtered and rinsed with EtOH. The process was repeated twice as more product precipitated form the filtrate. The combined solids afforded 1.75 g (60%) of 50a.
step 2—A suspension of 50a (1.75 g, 6.25 mmol) and Pd/C (10%, 250 mg) in 5:1 MeOH/1,4-dioxane (120 mL) was stirred under a H₂ atmosphere (balloon pressure) at RT for 72 h. The catalyst was filtered, and the filter cake was rinsed with MeOH. The filtrate was evaporated and the residue was purified via SiO₂ chromatography eluting with hexane/EtOAc to afford 0.340 g (22%) of 50b.
step 3—To a solution of 50b (0.34 g, 1.38 mmol) and 4 mL of H₂O was added a solution of KOH (0.155 g, 2.76 mmol) and 0.5 mL of H₂O. The mixture was stirred at 40° C. for 24 h them evaporated. The residue was partitioned between water and EtOAc. The aqueous layer was separated and adjusted to pH 2 with con HCl. The resulting precipitate washed with H₂O and acetone and dried to afford 0.235 g (78%) of 50c.
step 4—To a suspension of 40 (0.03 g, 0.0878 mmol), 50c (0.021 g, 0.0966 mmol), EDCl (0.020 g 0.105 mmol), HOBT monohydrate (0.016 g, 0.105 mmol), DMF (50 μL) and DCM (0.75 mL) was added diisopropylamine (70 μL, 0.4 mmol). The resulting solution was stirred at RT for 16 h. The resulting solution was partitioned between H₂O and EtOAc. The aqueous layer was twice extracted with EtOAc and the combined EtOAc extracts dried (Na₂SO₄), filtered and evaporated, The residue was purified by SiO₂ chromatography eluting with a gradient of 100% DCM to a linear gradient to a 1:1 mixture of DCM/(DCM/MeOH/NH₄Cl; 60/10/1), followed by isocratic elution with the 1:1 mixture for 10 min at a flow rate of 15 mL/min. to afford 0.0344 g (72.3%) of II-1.

### N-{(S)-3-[5-(3,5-Dimethyl-1-pyridazin-3-yl-1H-pyrazole-4-carbonyl)-hexahydro-pyrrolo[3,4-c]pyrrol-2-yl]-1-phenyl-propyl}-isobutyramide (II-2)

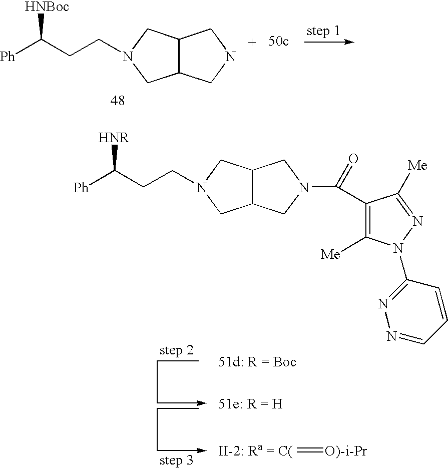

The amine 50 was coupled to 50c by the procedure described in step 5 of example 1. Steps 2 and 3 were carried out by the procedures described in steps 4 and 5 of example 3 except in step 5, cyclopentanecarbonyl chloride was replaced with iso-butyryl chloride to afford II-2.

### EXAMPLE 4

### Cyclopentanecarboxylic Acid [(S)-3-[5-(1-cyclohexyl-3,5-dimethyl-1H-pyrazole-4-carbonyl)-hexahydro-pyrrolo[3,4-c]pyrrol-2-yl]-1-(3-fluoro-phenyl)-propyl]-amide (II-9)

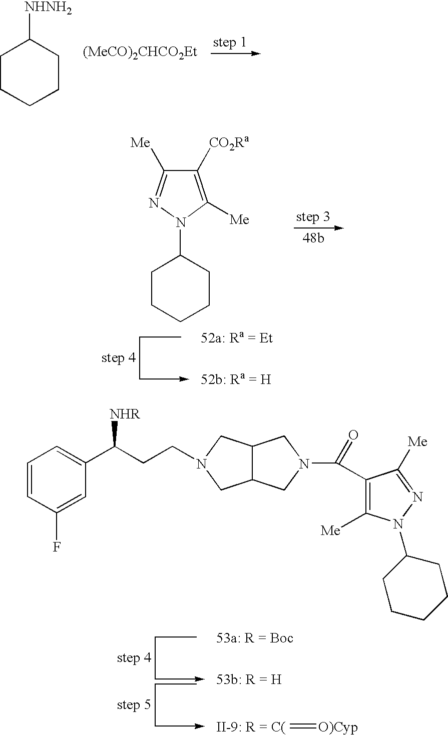 step 1 Ethyl diacetoacetate (2.3 mL, 14.7 mmol) was added at RT to a solution of cyclohexylhydrazine hydrochloride (2.0 g, 13.3 mmol) in a 8:5 mixture of MeOH/water (65 mL). The resulting mixture was vigorously stirred at RT for 18 h and then evaporated. The residue was purified via SiO₂ chromatography (hexane/EtOAc) to afford 1.6 g (48%) of 52a.
step 2—To a solution of 52a (1.6 g, 6.391 mmol) and EtOH (12 mL) was added a solution of KOH (1.076 g, 19.17 mmol) and H₂O (3 mL). The resulting solution was stirred at RT for 72 h. then heated at 50° C. for an additional 24 h. The resulting solution was cooled to RT and evaporated. The residue was partitioned between H₂O and EtOAc. The aqueous layer was adjusted to pH 2 with con HCl and the resulting precipitate filtered, rinsed with H₂O and dried to afford 1.34 g (94.3%) of 52b.
step 3—To a suspension of 50 (0.150 g, 0.413 mmol), 52b (0.11 g, 0.495 mmol), EDCl (0.095 g 0.495 mmol), HOBT monohydrate (0.076 g, 0.495 mmol) and DCM (3.0 mL) was added diisopropylamine (0.11 mL, 0.619 mmol). The resulting solution was stirred at RT for 24 then partitioned between H₂O and DCM. The aqueous layer was back-extracted with DCM and the combined organic layers dried (Na₂SO₄) and evaporated. The residue was purified by SiO₂ chromatography eluting with a gradient of 100% DCM for 1 min followed by a linear gradient to a 6:4 mixture of DCM/(DCM/MeOH/NH₄Cl; 60/10/1) over 20 min, followed by isocratic elution with the 6:4 mixture for 10 min at a flow rate of 25 mL/min. to afford 0.150 g (64%) of 53a.
step 4—A solution of 53a, TFA (2 mL) and DCM (2 mL) was stirred at RT for 18 h and evaporated. The residue was purified by SiO₂ chromatography eluting with a gradient of 100% DCM for 1 min followed by a linear gradient to a 6:4 mixture of DCM/(DCM/MeOH/NH₄Cl; 60/10/1) over 20 min, followed by isocratic elution with the 6:4 mixture for 10 min at a flow rate of 25 mL/min. to afford 0.09 g (73%) of 53b.
step 5—To a solution of 53b, pyridine (0.1 mL) and DCM (0.5 mL) was added cyclopentylcarbonyl chloride (20 μL, 0.128 mmol). The resulting mixture was stirred at RT for 4 d then quenched by the addition of MeOH (1 mL). The solution was stirred for 1 h and evaporated. The residue was purified by SiO₂ chromatography eluting with a gradient of 100% DCM for 1 min followed by a linear gradient to a 6:4 mixture of DCM/(DCM/MeOH/NH₄Cl; 60/10/1) over 20 min, followed by isocratic elution with the 6:4 mixture for 10 min at a flow rate of 15 mL/min. to afford 0.031 g (73%) of II-9

N—[(S)-3-[5-(1-Cyclohexyl-3,5-dimethyl-1H-pyrazole-4-carbonyl)-hexahydro-pyrrolo[3,4-c]pyrrol-2-yl]-1-(3-fluoro-phenyl)-propyl]-acetamide (II-7) was prepared by the procedure described in the present example except in step 5, acetyl chloride was used in place of cyclopentanecarbonyl chloride.

Tetrahydro-furan-3-carboxylic acid [(S)-3-[5-(1-cyclohexyl-3,5-dimethyl-1H-pyrazole-4-carbonyl)-hexahydro-pyrrolo[3,4-c]pyrrol-2-yl]-1-(3-fluoro-phenyl)-propyl]-amide (II-8) was prepared by the procedure described in the present example except in step 5, tetrahydro-furan-3-carbonyl chloride was used in place of cyclopentanecarbonyl chloride.

### EXAMPLE 5

### Cyclopentanecarboxylic Acid [(S)-3-[5-(1-cyclobutyl-3,5-dimethyl-1H-pyrazole-4-carbonyl)-hexahydro-pyrrolo[3,4-c]pyrrol-2-yl]-1-(3-fluoro-phenyl)-propyl]-amide (II-10)

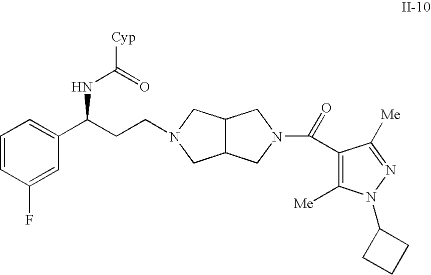

Cyclobutylhydrazine—A mixture of cyclobutanone ((2.0 g, 28.5 mmol) and t-butylcarbazate (3.77 g, 28.5 mmol) in hexane (50 mL) was heated to reflux and stirred for 1 h, then cooled to RT and stirred overnight. The precipitate formed (3.4 g) was filtered and rinsed with hexane. The solid was then dissolved in a solution of BH₃.Me₂S (2M in THF, 16 mL) and the mixture was stirred at RT for 1 h. The reaction was evaporated and the residue was taken up in THF, the insoluble material was filtered off and rinsed with THF (50 mL). The filtrate was evaporated affording 1.54 g (80%) of cyclobutylhydrazine which was used in the next step without further purification.

1-Cyclobutyl-3,5-dimethyl-1H-pyrazole-4-carboxylic acid was prepared from cyclobutylhydrazine by the procedures described in steps 1 and 2 of example 4.

Cyclopentanecarboxylic acid [(S)-3-[5-(1-cyclobutyl-3,5-dimethyl-1H-pyrazole-4-carbonyl)-hexahydro-pyrrolo[3,4-c]pyrrol-2-yl]-1-(3-fluoro-phenyl)-propyl]-amide (II-10) was prepared following the procedure steps outlined in steps 3 to 5 of example 4 except 1-cyclobutyl-3,5-dimethyl-1H-pyrazole-4-carboxylic acid was used in place of 1-cyclohexyl-3,5-dimethyl-1H-pyrazole-4-carboxylic acid.

N—[(S)-3-[-5-(1-Cyclobutyl-3,5-dimethyl-1H-pyrazole-4-carbonyl)-hexahydro-pyrrolo[3,4-c]pyrrol-2-yl]-1-(3-fluoro-phenyl)-propyl]-isobutyramide (II-11) was prepared from 1-cyclobutyl-3,5-dimethyl-1H-pyrazole-4-carboxylic acid following the steps described above except in step 5 isobutyryl chloride was used in place of cyclopentane carbonyl chloride.

Tetrahydro-furan-3-carboxylic acid [(S)-3-[-5-(1-cyclobutyl-3,5-dimethyl-1H-pyrazole-4-carbonyl)-hexahydro-pyrrolo[3,4-c]pyrrol-2-yl]-1-(3-fluoro-phenyl)-propyl]-amide (II-12) was prepared from 1-cyclobutyl-3,5-dimethyl-1H-pyrazole-4-carboxylic acid following the steps described above except in step 5 tetrahydrofuran-3-yl carbonyl chloride was used in place of cyclopentane carbonyl chloride.

### EXAMPLE 6

### Tetrahydro-furan-3-carboxylic acid [(S)-3-{5-[3,5-dimethyl-1-(5-trifluoromethyl-pyridin-2-yl)-1H-pyrazole-4-carbonyl]-hexahydro-pyrrolo[3,4-c]pyrrol-2-yl}-1-(3-fluoro-phenyl)-propyl]-amide (II-3)

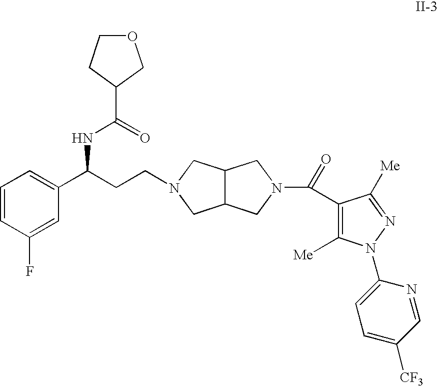

3,5-Dimethyl-1-(5-trifluoromethyl-pyridin-2-yl)-1H-pyrazole-4-carboxylic acid ethyl ester—Ethyl diacetoacetate (0.90 mL, 5.76 mmol) was added at RT to a mixture of 5-(trifluoromethyl)pyrid-2-ylhydrazine (1.0 g, 5.65 mmol) in a mixture of EtOH/HOAc (2/3, 25 mL). The reaction was stirred at RT for 2 weeks and then evaporated. The residue was evaporated with toluene, it was then purified via SiO₂ chromatography (hexane/EtOAc) affording 1.24 g (70%) the desired product.

The title compound was prepared by the procedure described in example 4 except in step 3,3,5-dimethyl-1-(5-trifluoromethyl-pyridin-2-yl)-1H-pyrazole-4-carboxylic acid was used in place of 3,5-dimethyl-1-cyclohexyl-1H -pyrazole-4-carboxylic acid and in step 5, tetrahydrofuran-3-yl carbonyl chloride was used in place of cyclopentanecarbonyl chloride to afford II-3.

N—[(S)-3-{5-[3,5-Dimethyl-1-(5-trifluoromethyl-pyridin-2-yl)-1H-pyrazole-4-carbonyl]-hexahydro-pyrrolo[3,4-c]pyrrol-2-yl}-1-(3-fluoro-phenyl)-propyl]-amide (II-4) was prepared by the procedure described in example 4 except in step 5 except cyclopentanecarbonyl chloride was replaced by acetyl chloride.

[(S)-3-{5-[3,5-Dimethyl-1-(5-trifluoromethyl-pyridin-2-yl)-1H-pyrazole-4-carbonyl]-hexahydro-pyrrolo[3,4-c]pyrrol-2-yl}-1-(3-fluoro-phenyl)-propyl]-carbamic acid methyl ester (II-6) was prepared by the procedures described in example 4 except in step 5, cyclopentanecarbonyl chloride was replaced by methyl chloroformate.

### EXAMPLE 7

### 1-Acetyl-azetidine-3-carboxylic acid [(S)-3-{5-[3,5-dimethyl-1-(6-trifluoromethyl-pyridazin-3-yl)-1H-pyrazole-4-carbonyl]-hexahydro-pyrrolo[3,4-c]pyrrol-2-yl}-1-(3-fluoro-phenyl)-propyl]-amide (II-17)

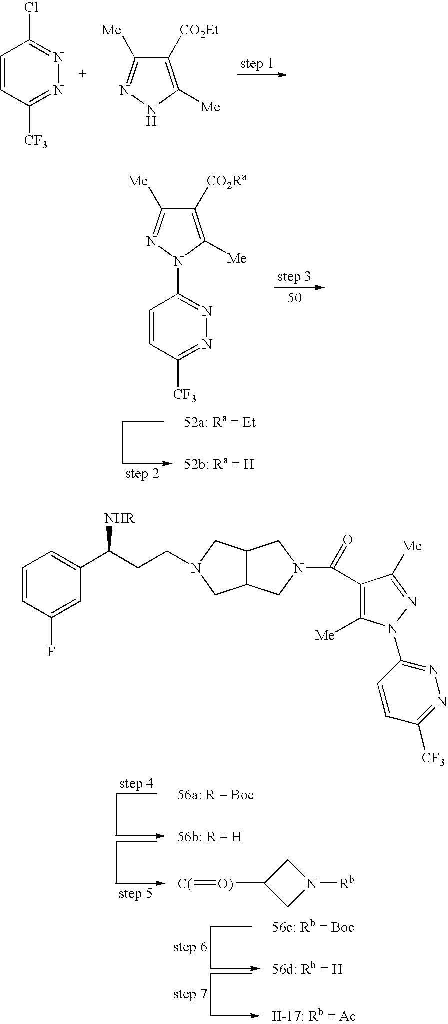 step 1—To a solution of 3,5-dimethyl-1H-pyrazole-4-carboxylic acid ethyl ester (0.2 g, 1.19 mmol) in DMF (10 mL) cooled to 0° C. was added sequentially NaH (60% in mineral oil, 72 mg, 1.78 mmol) and 3-chloro-6-trifluoromethyl-pyridazine (0.22 g, 1.21 mmol; Tetrahedron 1999 55:15067-15070). The resulting mixture was stirred at RT for 3 h then partitioned between EtOAc and saturated aqueous NH₄Cl. The layers were separated and the aqueous layer was extracted twice with EtOAc. The combined extracts were dried (Na₂SO₄), filtered and evaporated. The residue was purified via SiO₂ chromatography eluting with hexane/EtOAc to afford 0.228 g (62%) of 55a.
Steps 2-4 were carried out as described in steps 2-4 of example 4 except 3,5-dimethyl-1-(6-trifluoromethyl-pyridazin-3-yl)-1H-pyrazole-4-carboxylic acid ethyl ester was used in step 2 in place of 52b to ultimately afford 56b.
Step 5 was carried out by EDCI-mediated coupling as described in step 4 of example 3 except azetidine-1, 3-dicarboxylic acid mono-tert-butyl ester was used as the carboxylic acid in place of 50c.
steps 6 and 7—A solution of 56c (55 mg, 0.077 mmol) and 1:1 TFA/DCM (2 mL) was stirred at RT overnight. The resulting solution was evaporated and the residue dissolved in DCM (2 mL) and MP-carbonate resin was added to neutralize the solution. The resin was filtered off and rinsed with DCM. The filtrate was evaporated and the residue was dissolved in a 4:2:1 mixture DCM/pyridine/Ac₂O (1.75 mL). The reaction was stirred overnight and then quenched by addition of MeOH (1 mL), stirred 1 h and evaporated. The residue was purified via SiO₂ chromatography eluting with DCM/MeOH/NH₄OH (60/10/1) to afford 0.024 g (48%) of II-17.

Tetrahydro-furan-3-carboxylic acid [(S)-3-{5-[3,5-dimethyl-1-(6-trifluoromethyl-pyridazin-3-yl)-1H-pyrazole-4-carbonyl]-hexahydro-pyrrolo[3,4-c]pyrrol-2-yl}-1-(3-fluoro-phenyl)-propyl]-amide (II-16) was prepared from 56b utilizing the procedure described in the present example except step 5 was carried out using tetrahydrofuran-3-ylcarbonyl chloride in place of cyclopentanecarbonyl chloride as described in step 5 of example 4 and steps 6 and 7 were omitted.

Cyclopentanecarboxylic acid [(S)-3-{5-[3,5-dimethyl-1-(6-trifluoromethyl-pyridazin-3-yl)-1H-pyrazole-4-carbonyl]-hexahydro-pyrrolo[3,4-c]pyrrol-2-yl}-1-(3-fluoro-phenyl-propyl]amide (II-13) was prepared from 56b utilizing the procedure described in the present example except step 5 was carried out using cyclopentanecarbonyl chloride as described in step 5 of example 4 and steps 6 and 7 were omitted.

N—[(S)-3-{5-[3,5-Dimethyl-1-(6-trifluoromethyl-pyridazin-3-yl)-1H-pyrazole-4-carbonyl]-hexahydro-pyrrolo[3,4-c]pyrrol-2-yl}-1-(3-fluoro-phenyl)-propyl]-acetamide (II-14) was prepared from 56b utilizing the procedure described in the present example except step 5 was carried out using acetyl chloride in place of cyclopentanecarbonyl chloride as described in step 5 of example 4 and steps 6 and 7 were omitted.

N—[(S)-3-{5-[3,5-Dimethyl-1-(6-trifluoromethyl-pyridazin-3-yl)-1H-pyrazole-4-carbonyl]-hexahydro-pyrrolo[3,4-c]pyrrol-2-yl}-1-(3-fluoro-phenyl)-propyl]-acetamide (II-15) was prepared from 56b utilizing the procedure described the present example except step 5 was carried out using isobutryl chloride in place of cyclopentanecarbonyl chloride as described in step 5 of example 4 and steps 6 and 7 were omitted.

1-Acetyl-azetidine-3-carboxylic acid [(S)-3-{5-[3,5-dimethyl-1-(6-methyl-pyridazin-3-yl)-1H-pyrazole-4-carbonyl]-hexahydro-pyrrolo[3,4-c]pyrrol-2-yl}-1-(3-fluoro-phenyl)-propyl]-amide (II-24) was prepared in analogous fashion to II-17 except in step 1,3-chloro-6-trifluoromethyl-pyridazine was replaced with 3-chloro-6-methyl-pyridazine

N—[(S)-3-{(5-[3,5-Dimethyl-1-(6-methyl-pyridazin-3-yl)-1H-pyrazole-4-carbonyl]-hexahydro-pyrrolo[3,4-c]pyrrol-2-yl}-1-(3-fluoro-phenyl)-propyl]-acetamide (II-20) was prepared in analogous fashion except in step 1 3-chloro-6-trifluoromethyl-pyridazine was replaced with 3-chloro-6-methyl-pyridazine and step 5 was carried out using acetyl chloride in place of cyclopentanecarbonyl chloride as described in step 5 of example 4 and steps 6 and 7 were omitted.

Tetrahydro-furan-3-carboxylic acid [(S)-3-{5-[3,5-dimethyl-1-(6-methyl-pyridazin-3-yl)-1H-pyrazole-4-carbonyl]-hexahydro-pyrrolo[3,4-c]pyrrol-2-yl}-1-(3-fluoro-phenyl)-propyl]-amide (II-21) was prepared as described for II-20 (supra) except in step 5 acetyl chloride was replaced with tetrahydrofuran-3-carbonyl chloride.

N—[(S)-3-{5-[3,5-Dimethyl-1-(6-methyl-pyridazin-3-yl)-1H-pyrazole-4-carbonyl]-hexahydro-pyrrolo[3,4-c]pyrrol-2-yl}-1-(3-fluoro-phenyl)-propyl]-isobutyramide (II-22) was prepared as described for II-20 (supra) except in step 5 acetyl chloride was replaced with isobutyryl chloride.

Cyclopentanecarboxylic acid [(S)-3-{5-[3,5-dimethyl-1-(6-methyl-pyridazin-3-yl)-1H-pyrazole-4-carbonyl]-hexahydro-pyrrolo[3,4-c]pyrrol-2-yl}-1-(3-fluoro-phenyl)-propyl]-amide (II-23) as prepared as described for II-20 (supra) except in step 5 acetyl chloride was replaced with cyclopentylcarbonyl chloride.

### EXAMPLE 8

### 1-Acetyl-azetidine-3-carboxylic Acid [(S)-3-[5-(3,5-dimethyl-1-pyrazin-2-yl-1H-pyrazole-4-carbonyl)-hexahydro-pyrrolo[3,4-c]pyrrol-2-yl]-1-(3-fluoro-phenyl)-propyl]-amide (III-25)

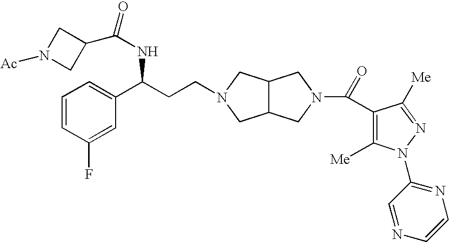

3,5-Dimethyl-1-pyrazin-2-yl-1H-pyrazole-4-carboxylic acid—To a solution of 3,5-dimethyl-1H-pyrazole-4-carboxylic acid ethyl ester (1 g, 5.95 mmol) in DMF (20 mL) cooled to 0° C. was added portionwise NaH (60% in mineral oil, 171 mg, 7.13 mmol). After hydrogen evolution ceased, 2-chloro-pyrazine (0.64 mL, 7.13 mmol) was added and the reaction was stirred at 50° C. for 24 h. The reaction mixture was cooled to RT, partitioned between EtOAc and saturated NH₄Cl. The aqueous layer was extracted twice with EtOAc. The combined organic layers were dried (Na₂SO₄), filtered and evaporated. The residue was purified via SiO₂ chromatography eluting with hexane/EtOAc to afford 0.64 g (44%) of 3,5-dimethyl-1-pyrazin-2-yl-1H -pyrazole-4-carboxylic acid ethyl ester. The ethyl ester was hydrolyzed to the corresponding acid with KOH in aqueous EtOH as described in step 3 of example 3.

The title compound was prepared by the procedures described in steps 3-7 of example 7 except in step 3, 3,5-dimethyl-1-(6-trifluoromethyl-pyridazin-3-yl)-1H-pyrazole-4-carboxylic acid was replaced with 3,5-dimethyl-1-pyrazin-2-yl-1H-pyrazole-4-carboxylic acid to afford II-25

N—[(S)-3-[5-(3,5-Dimethyl-1-pyrazin-2-yl-1H-pyrazole-4-carbonyl)-hexahydro-pyrrolo[3,4-c]pyrrol-2-yl]-1-(3-fluoro-phenyl)-propyl]-acetamide (II-26), N—[(S)-3-[5-(3,5-dimethyl-1-pyrazin-2-yl-1H-pyrazole-4-carbonyl)-hexahydro-pyrrolo[3,4-c]pyrrol-2-yl]-1-(3-fluoro-phenyl)-propyl]-isobutyramide (II-27), cyclopentanecarboxylic acid [(S)-3-[5-(3,5-dimethyl-1-pyrazin-2-yl-1H-pyrazole-4-carbonyl)-hexahydro-pyrrolo[3,4-c]pyrrol-2-yl]-1-(3-fluoro-phenyl)-propyl]-amide (II-28) and tetrahydro-furan-3-carboxylic acid [(S)-3-[5-(3,5-dimethyl-1-pyrazin-2-yl-1H-pyrazole-4-carbonyl)-hexahydro-pyrrolo[3,4-c]pyrrol-2-yl]-1-(3-fluoro-phenyl)-propyl]-amide (II-29) were prepared by the procedures outlined above and example 7 (step 5) except in step 5, azetidine-1,3-dicarboxylic acid mono-tert-butyl ester was replaced with acetyl chloride, isobutyryl chloride, cyclopentane carbonyl chloride and tetrahydro-furan-3-carbonyl chloride used, respectively, were used to form the amide as described in step 5 of example 4, and steps 6 and 7 of example 7 were omitted.

### EXAMPLE 9

### (S)-1-(5-{5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yl)-pyrrolidine-2-carboxylic Acid Ethyl Ester (I-23) and (S)-1-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yl)-pyrrolidine-2-carboxylic Acid (I-24)

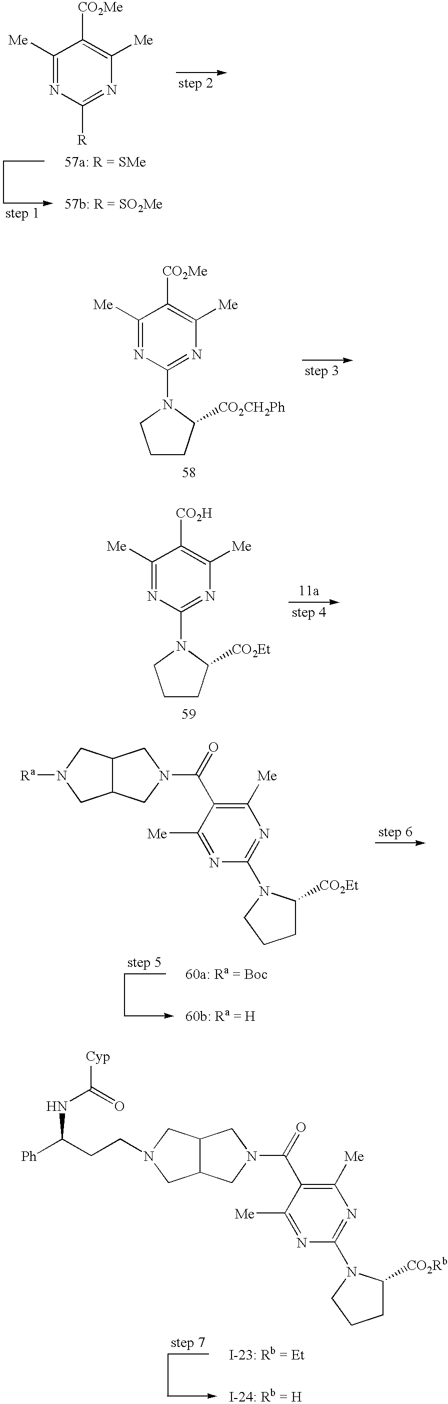 step 1—A mixture of 57a (6.04 g) and MCPBA (77%, 14.0 g) in DCM (150 mL) was stirred overnight at RT. The white precipitate was filtered and the filtrate was partitioned between EtOAc and water. The organic layer was separated, washed with saturated NaHCO₃, water and brine, dried (MgSO₄), filtered and evaporated. The residue was purified via SiO₂ chromatography eluting with hexane/EtOAc to afford 5.96 g of 57b.
step 2—A mixture of 57 (2.0 g, 8.18 mmol), (S)-pyrrolidine-2-carboxylic acid benzyl ester hydrochloride (2.0 g), TEA (3.4 mL) and MeCN (10 mL) was heated at 120° C. in a laboratory microwave for 30 min. The mixture was partitioned between EtOAc and water, the organic layer was separated and washed with water and brine, dried (MgSO₄), filtered and evaporated. The residue was purified via SiO₂ chromatography eluting with hexane/EtOAc to afford 1.86 (62%) of 58.
step 3—A mixture of 58 (1.85 g, 5.01 mmol) and LiOH.H₂O (256 mg) in 1:1 MeOH/water (20 mL) was stirred at RT for 2 h and a second aliquot of LiOH.H₂O (378 mg) was added. The reaction was stirred at 70° C. overnight and concentrated. The residue was taken up in EtOH (100 mL). and con H₂SO₄(2 mL) was added. The reaction was stirred at 75° C. overnight, concentrated and the residue containing 59 was used for next step without further purification.
step 4—To a mixture of 59 (5.01 mmol) and TEA (3 mL) in DCM (25 mL) was added 11b (1.15 g) followed by TBTU (1.9 g). The reaction was stirred for 3 h and partitioned between EtOAc and water. The organic layer was separated, washed with water and brine, dried (MgSO₄), filtered and evaporated. The residue was purified via SiO₂ chromatography eluting with DCM/MeOH/NH₄OH to afford 2.04 g of 60a.
step 5—A mixture of 60a (2.04 g, 4.18 mmol) and TFA (3.5 mL) in DCM (25 mL) was stirred at RT for 3 h. It was evaporated, concentrated, and thrice redissolved in DCM and re-evaporated. The resulting residue containing 60b was dried under a high vacuum and used in the next step.
step 6 was carried by reductive amination of 60b with cyclopentanecarboxylic acid ((S)-3-oxo-1-phenyl-propyl)-amide as described in step 5 of example 2 to afford I-23.
step 7—A mixture of I-23 (33 mg) and LiOH.H₂O (10 mg) in 1:1 MeOH/water (2 mL) was stirred at RT overnight. The reaction was then concentrated and the residue was purified via preparative TLC developed with DCM/MeOH/NH₄OH to afford I-24.

(S)—-[5-(-5-{3-[(3-Chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-yl]-pyrrolidine-2-carboxylic acid ethyl ester I-25 was prepared in similar fashion except in step 6, 60b was alkylated with 4,4-difluoro-cyclohexanecarboxylic acid (3-chloro-4-methyl-phenyl)-(3-chloro-propyl)-amide as described in step 3 of example 13 instead of a reductive amination used above to afford I-25. The corresponding acid I-29 was prepared from I-25 by the procedure described in step 7 of the present example.

### EXAMPLE 10

### 2-(5-{-5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yloxy)-2-methyl-propionic Acid Methyl Ester (I-26); 2-(5-{(5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-2-methyl-propionic Acid (I-32) and Cyclopentanecarboxylic Acid {(S)-3-[5-(2-methoxy-4,6-dimethyl-pyrimidine-5-carbonyl)-hexahydro-pyrrolo[3,4-c]pyrrol-2-yl]-1-phenyl-propyl}-amide (I-19)

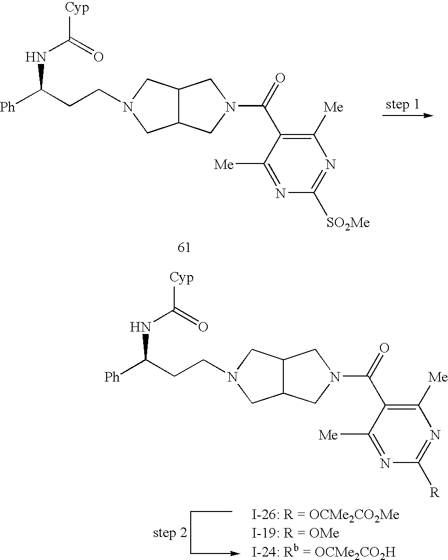

61 was prepared by acylation of 11b with 2-methanesulfonyl-4,6-dimethyl-pyrimidine-5-carboxylic acid as described in step 4 of example 9 followed by removal of the BOC protecting group (TFA/DCM) and reductive amination with cyclopentanecarboxylic acid ((S)-3-oxo-1-phenyl-propyl)-amide (CAS Reg. No. 135868-78-0) as described in step 5 of example 2.

step 1—A mixture of 61 (209 mg, 0.374 mmol), 2-hydroxy-2-methyl-propionic acid methyl ester (0.5 mL) and K₂CO₃ (500 mg, 1.53 mmol) in DMF (2.0 mL) was heated at 70° C. overnight. The reaction mixture was cooled to RT and partitioned between water and EtOAc. The organic layer was separated and washed 3 times with water and once with brine, dried (MgSO₄), filtered and evaporated. The residue was purified via SiO₂ chromatography eluting with DCM/MeOH/NH₄OH to afford 0.016 g of I-26 and 0.067 g of I-19.

Step 2 was carried out as described in step 7 of example 9 to afford I-32.

2-(5-{5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid methyl ester (I-4) and 2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid; trifluoro-acetic acid salt were prepared by the procedure described in the present example except in step 2,2-hydroxy-2-methyl-propionic acid methyl ester was replaced with 2-hydroxy-propionic acid methyl ester to afford I-4 which was hydrolyzed to afford I-6.

4-(5-{5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yloxy)-benzoic acid benzyl ester and 4-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-benzoic acid trifluoro-acetate salt were by the procedure described in the present example except in step 2,2-hydroxy-2-methyl-propionic acid methyl ester was replaced with 4-hydroxy-benzoic acid benzyl ester to afford I-7 which was hydrolyzed to afford I-8.

3-(5-{5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yloxy)-benzoic acid ethyl ester and 3-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-benzoic acid, trifluoro acetic acid salt were prepared by the procedure described in the present example except in step 2,2-hydroxy-2-methyl-propionic acid methyl ester was replaced with 3-hydroxy-benzoic acid ethyl ester to afford I-9 which was hydrolyzed to afford I-10.

(R)-(5-{5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yloxy)-phenyl-acetic acid methyl ester and (R)-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-phenyl-acetic acid were prepared by the procedure described in the present example except in step 2,2-hydroxy-2-methyl-propionic acid methyl ester was replaced with (R)-hydroxy-phenyl-acetic acid methyl ester to afford I-12 which was hydrolyzed to afford I-18. (S)-(5-{-5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yloxy)-phenyl-acetic acid methyl ester (I-16) and (S)-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-phenyl-acetic acid (I-17) were prepared in analogous manner from (S)-hydroxy-phenyl-acetic acid methyl ester

(R)-2-(5-{5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid ethyl ester and (R)-2-(5-{-5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid were prepared by the procedure described in the present example except in step 2,2-hydroxy-2-methyl-propionic acid methyl ester was replaced with (R)-2-hydroxy-propionic acid ethyl ester to afford I-22 which was hydrolyzed to afford I-20. (S)-2-(5-{5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid methyl ester (I-13) and (S)-2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid (I-15) were prepared in analogous manner from (S)-hydroxy-propionic acid methyl ester.

2-(5-{-5-[(S)-3-(Cyclopentanecarbonyl-amino)-3-phenyl-propyl]-hexahydro-pyrrolo[3,4-c]pyrrole-2-carbonyl}-4,6-dimethyl-pyrimidin-2-yloxy)-butyric acid ethyl ester and 2-(5-{-5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-butyric acid were prepared by the procedure described in the present example except in step 2, (R)-2-hydroxy-propionic acid ethyl ester was replaced with 2-hydroxy-butyric acid ethyl ester to afford I-22 which was hydrolyzed to afford I-20.

### EXAMPLE 11

### [4-(5-{3-[(1-Acetyl-piperidine-4-carbonyl)-(3-chloro-4-methyl-phenyl)-amino]-propyl}-hexahydro-pyrrolo[3,4-c]pyrrole-2-carbonyl)-3,5-dimethyl-pyrazol-1-yl]-acetic acid trifluoro acetate (II-18)

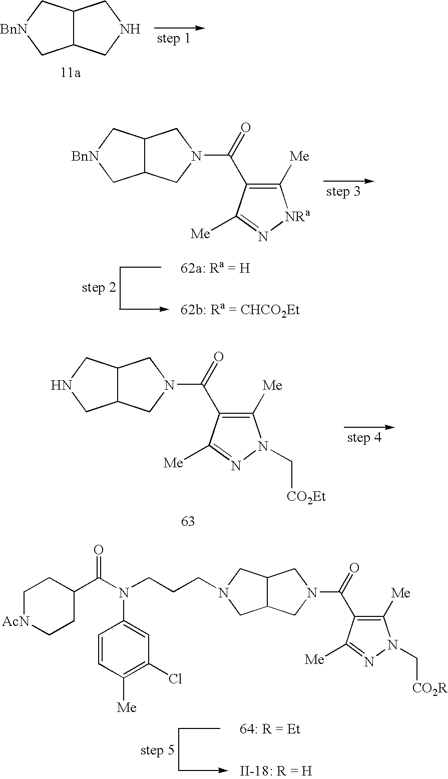 step 1—A mixture of 3,5-dimethyl-1H-pyrazole-4-carboxylic acid (820 mg; CAS Reg No. 113808-86-9), 11a (2.42 g), TEA (2.2 mL) and PyBOP (5.84 g) in DMF (15 mL) was stirred at RT overnight. The reaction was quenched by addition of water and partitioned between water and EtOAc. The organic layer was separated and washed 3 times with water and once with brine, dried (MgSO₄), filtered and evaporated. The residue was purified via SiO₂ chromatography eluting with DCM/MeOH/NH₄OH to afford 0.829 g of 62a.
step 2—To a solution of 62a (115 mg, 0.354 mmol) in DMF (2 mL) at RT was added NaH (60% in mineral oil, 30 mg) and the resulting mixture was stirred for 25 min. Chloro-acetic acid methyl ester (0.1 mL, 1.14 mmol) was added and the reaction was stirred overnight. The reaction mixture was partitioned between water and EtOAc and the organic layer was separated and washed with water and brine, dried (MgSO₄), filtered and evaporated. The residue was purified via SiO₂ chromatography eluting with DCM/MeOH/NH₄OH to afford 0.130 g of 62b.
step 3—Debenzylation of 62b was carried out as described in step 6 of example 2.
step 4—A mixture of 63 (130 mg, 0.424 mmol), 1-acetyl-piperidine-4-carboxylic acid (3-chloro-4-methyl-phenyl)-(3-chloro-propyl)-amide (190 mg), NaI (63 mg), and DIPEA (0.2 mL) in MeCN (3 mL) was heated to 160° C. in a laboratory microwave for 30 minutes. The resulting solution was then partitioned between water and EtOAc and the organic layer washed with water and brine, dried (MgSO₄), filtered and evaporated. The residue was purified via SiO₂ chromatography eluting with DCM/MeOH/NH₄OH to afford 0.112 g of 64.
step 5—Hydrolysis of the ethyl ester of 64 was carried out by the procedure described in step 7 of example 9 to afford II-18.

(4-{5-[(S)-3-(Cyclopentanecarbonyl-amino)-3-phenyl-propyl]-hexahydro-pyrrolo[3,4-c]pyrrole-2-carbonyl}-3,5-dimethyl-pyrazol-1-yl)-acetic acid (II-19) was prepared analogously except the alkylation described in step 4 was replaced by a reductive amination with cyclopentanecarboxylic acid (3-oxo-1-phenyl-propyl)-amide and 63. Hydrolysis of the resulting ester was carried out as described in step 5 of the present example.

### EXAMPLE 12

### (R)-2-[5-(5-{3-[(3-Chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-ylamino]-propionic Acid Ethyl Ester (I-34) and (R)-2-[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-ylamino]-propionic Acid (I-36)

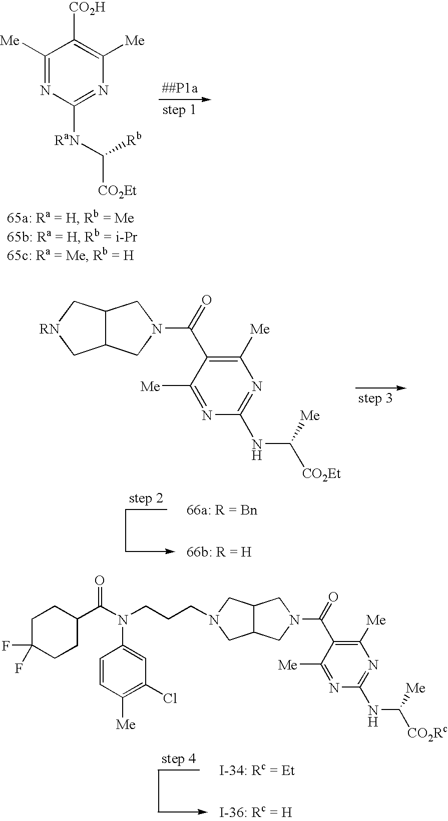

2-((R)-1-Ethoxycarbonyl-ethylamino)-4,6-dimethyl-pyrimidine-5-carboxylic acid (65a) was prepared following the procedure described for 2-((S)-2-ethoxycarbonyl-pyrrolidin-1-yl)-4,6-dimethyl-pyrimidine-5-carboxylic acid (59) in steps 1-3 of example 9 except alanine benzyl ester was used in place of proline benzyl ester.

step 1—To a mixture of 65a (1.1 mmol) and 11a (1.1 mmol) in DCM (2 mL) was added TEA (2.2 mmol) followed by TBTU (392 mg, 1.21 mmol). The reaction was stirred for 90 min at RT then extracted with DCM. The organic layer was dried (Na₂SO₄), filtered and evaporated. The crude residue was purified via SiO₂ chromatography eluting with DCM/MeOH/NH₄OH to afford 66a.

step 2—A mixture of 66a (260 mg) and 20% Pd(OH)₂/C (160 mg) in EtOH was stirred in a Parr apparatus under H₂ atmosphere (50 psi) overnight and then a few more hours at increased H₂ pressure (55 PSI). The mixture was filtered and the filtrate evaporated to afford 0.150 g of 66b which was used without further purification.

step 3—A mixture of 66b, 4,4-difluoro-cyclohexanecarboxylic acid (3-chloro-4-methyl-phenyl)-(3-chloro-propyl)-amide (220 mg, 0.962 mmol), DIPEA (220 μL) and KI (60 mg) in MeCN was stirred at 120° C. in a laboratory microwave for 30 min. It was poured into water and extracted with EtOAc. The combined organic extracts were dried (Na₂SO₄), filtered and evaporated. The crude residue was purified via SiO₂ chromatography eluting with DCM/MeOH/NH₄OH to afford 0.181 g of I-34.

step 4—A mixture of I-34 (141 mg, 0.2 mmol) and LiOH.H₂O (20 mg, 0.4 mmol) in MeOH with a few drops of water was stirred at RT overnight. The solvent was evaporated and the residue was purified via SiO₂ TLC developed with DCM/MeOH/NH₄OH to afford 0.104 g of I-36: mp 159.5-161.3° C.

I-28 and I-30 were prepared as described in steps 1-4 of the present example except in step 1 65a was replaced with 2-((R)-1-ethoxycarbonyl-2-methyl-propylamino)-4,6-dimethyl-pyrimidine-5-carboxylic acid (65b). 65b was prepared following the procedure described for 2-((S)-2-ethoxycarbonyl-pyrrolidin-1-yl)-4,6-dimethyl-pyrimidine-5-carboxylic acid (59) in steps 1-3 of example 9 except valine ethyl ester was used in place of proline benzyl ester.

I-37 and I-38 were prepared as described in steps 1-4 of the present example except in step 1 65a was replaced with 65c. 65c was prepared following the procedure described for 2-((S)-2-ethoxycarbonyl-pyrrolidin-1-yl)-4,6-dimethyl-pyrimidine-5-carboxylic acid (59) in steps 1-3 of example 9 except N-methylglycine ethyl ester was used in place of proline benzyl ester.

I-28 and I-30 were prepared as described in steps 1-4 of the present example except in step 1 65a was replaced with 65b. 65b was prepared following the procedure described for 2-((S)-2-ethoxycarbonyl-pyrrolidin-1-yl)-4,6-dimethyl-pyrimidine-5-carboxylic acid (59) in steps 1-3 of example 9 except valine ethyl ester was used in place of proline benzyl ester.

I-33 was prepared in similar fashion except step 3 was a reductive amination as described in step 5 of example 2 except in which 47a was replaced with cyclopentanecarboxylic acid ((S)-3-oxo-1-phenyl-propyl)-amide to afford I-33. I-35 was prepared from I-33 by the procedure described in step 4 of the present example to afford I-35.

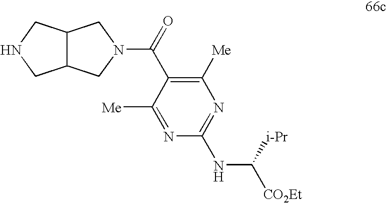

I-27 and I-31 were prepared as described for I-33 and I-35 except 65a was replaced with 65b to afford 66c. I-27 was obtained by reductive amination of 66c and cyclopentanecarboxylic acid ((S)-3-oxo-1-phenyl-propyl)-amide (CAS Reg. No. 872001-31-5). I-31 was prepared by hydrolysis of I-27.

### EXAMPLE 13

### [5-(-5-{3-[(3-Chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-2-oxo-2H-pyridin-1-yl]-acetic Acid Ethyl Ester (III-9) and [5-(5-{3-[(3-Chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-2-oxo-2H-pyridin-1-yl]-acetic Acid (III-10)

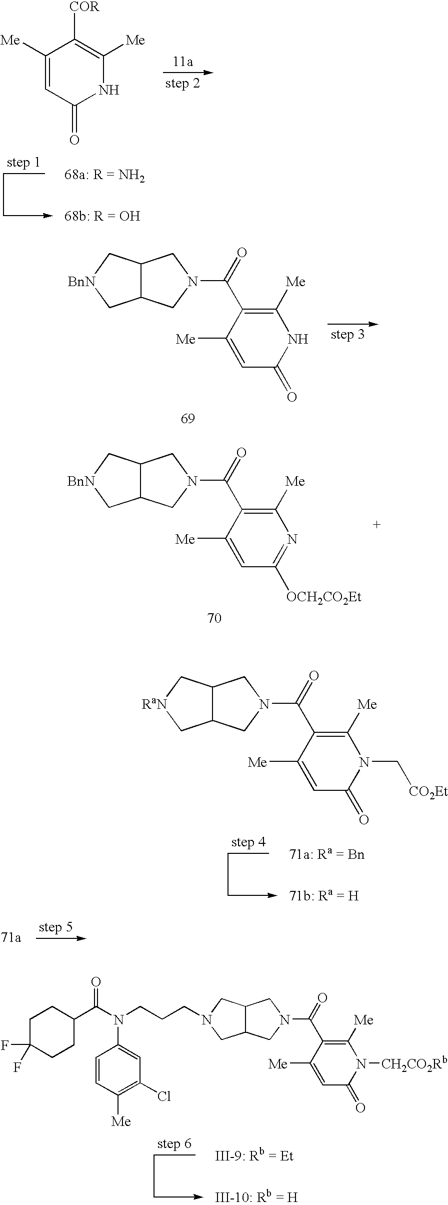 step 1—A solution of NaNO₂ (8.28 g, 0.12 mol) in H₂SO₄ (15% in water, 160 mL) was added dropwise at a rate of 1 drop per second to a solution of 2,4-dimethyl-6-oxo-1,6-dihydro-pyridine-3-carboxylic acid amide (68a; 16.6 g, 0.1 mol) in H₂SO₄ (15% in water, 80 mL) cooled in a ice-water bath. The reaction was stirred overnight then poured into ice-cold water (1.5 L). The precipitate was filtered and washed with water (400 mL), Et₂O (350 mL) and dried to afford 16.7 g (100%) of 68b.
step 2—5-(5-Benzyl-hexahydro-pyrrolo[3,4-c]pyrrole-2-carbonyl)-4,6-dimethyl-1H-pyridin-2-one (69) was prepared by condensing 11a and 68b following the procedure described in step 1 of example 12.
step 3—A mixture of 69 (2.8 g, 8 mmol), ethyl bromo-acetate (1.6 g) and K₂CO₃ (3.29 g) in MeCN was stirred at RT overnight. The reaction was quenched by the addition of water and was extracted 3 times with EtOAc. The combined organic extracts were dried (Na₂SO₄), filtered and evaporated. The crude residue was purified via SiO₂ chromatography eluting with DCM/MeOH/NH₄OH to afford 1.29 g of O-alkylated product 70 and 0.765 g of N-alkylated product 71a.
steps 4-6—Debenzylation of 71a was carried out as described in step 2 of example 12 to afford 71b. N-alkylation of 71b with 4,4-difluoro-cyclohexanecarboxylic acid (3-chloro-4-methyl-phenyl)-amide was carried out as described in step 3 of example 12. Hydrolysis of the carboxylic acid was carried out as described in step 4 of example 12.

[5-(5-{3-[(1-Acetyl-piperidine-4-carbonyl)-(3-chloro-4-methyl-phenyl)-amino]-propyl}-hexahydro-pyrrolo[3,4-c]pyrrole-2-carbonyl)-4,6-dimethyl-2-oxo-2H-pyridin-1-yl]-acetic acid ethyl ester (III-1) and [5-(-5-{3-[(1-Acetyl-piperidine-4-carbonyl)-(3-chloro-4-methyl-phenyl)-amino]-propyl}-hexahydro-pyrrolo[3,4-c]pyrrole-2-carbonyl)-4,6-dimethyl-2-oxo-2H-pyridin-1-yl]-acetic acid; compound with trifluoro-acetic acid (III-2) were prepared by the procedure described in the present example except the alkylation in step 5, 4,4-difluoro-cyclohexanecarboxylic acid (3-chloro-4-methyl-phenyl)-(3-chloro-propyl)-amide was replaced with 1-acetyl-piperidine-4-carboxylic acid (3-chloro-4-methyl-phenyl)-(3-chloro-propyl)-amide (CAS Reg No. 333985-70-9, I. Shinichi et al. WO2001025200) to afford III-1 which was hydrolyzed as described in step 6 to the corresponding acid III-2. The TFA salt of [5-(5-{3-[(1-acetyl-piperidine-4-carbonyl)-(3-chloro-4-methyl-phenyl)-amino]-propyl}-hexahydro-pyrrolo[3,4-c]pyrrole-2-carbonyl)-4,6-dimethyl-pyridin-2-yloxy]-acetic acid (III-4) was prepared analogously from the O-alkylated product 70 to afford III-4.

2-[5-(-5-{3-[(1-Acetyl-piperidine-4-carbonyl)-(3-chloro-4-methyl-phenyl)-amino]-propyl}-hexahydro-pyrrolo[3,4-c]pyrrole-2-carbonyl)-4,6-dimethyl-pyridin-2-yloxy]-propionic acid ethyl ester (III-6) and 2-[5-(5-{3-[(1-acetyl-piperidine-4-carbonyl)-(3-chloro-4-methyl-phenyl)-amino]-propyl}-hexahydro-pyrrolo[3,4-c]pyrrole-2-carbonyl)-4,6-dimethyl-pyridin-2-yloxy]-propionic acid (III-7) were prepared in analogous manner from the N-alkylated intermediate except in step 3 ethyl bromo-acetate was replaced with ethyl 1-bromo-propionate. III-6 was hydrolyzed to III-7.

4,4-Difluoro-cyclohexanecarboxylic acid (3-chloro-4-methyl-phenyl)-{3-[5-(2,4-dimethyl-6-oxo-1,6-dihydro-pyridine-3-carbonyl)-hexahydro-pyrrolo[3,4-c]pyrrol-2-yl]-propyl}-amide (III-8) and the TFA salt of 1-acetyl-piperidine-4-carboxylic acid (3-chloro-4-methyl-phenyl)-{3-[5-(2,4-dimethyl-6-oxo-1,6-dihydro-pyridine-3-carbonyl)-hexahydro-pyrrolo[3,4-c]pyrrol-2-yl]-propyl}-amide (III-5) were prepared as described above but the alkylation step, step 3 was omitted and 69 was debenzylated and converted to III-8 and III-5 by alkylation with 4,4-difluoro-cyclohexanecarboxylic acid (3-chloro-4-methyl-phenyl)-amide and 1-acetyl-piperidine-4-carboxylic acid (3-chloro-4-methyl-phenyl)-(3-chloro-propyl)-amide respectively.

### EXAMPLE 14

### 1-Cyclopentanecarbonyl-azetidine-3-carboxylic Acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide (III-19)

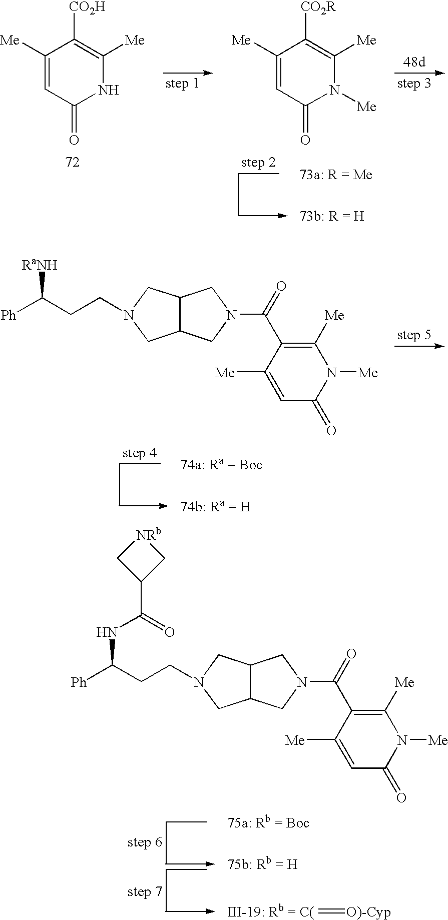 step 1—A mixture of 2,4-dimethyl-6-oxo-1,6-dihydro-pyridine-3-carboxylic acid (72; 560 mg, 3 mmol), MeI (1.28 g, 9 mmol) and Cs₂CO₃ (3.26 g, 10 mmol) in MeCN was stirred at RT overnight. The reaction was poured into water and extracted 3 times with EtOAc. The combined organic extracts were dried (Na₂SO₄), filtered and evaporated. The crude residue was purified via SiO₂ chromatography eluting with DCM/MeOH/NH₄OH to afford 0.460 g of 73a.
step 2-1,2,4-Trimethyl-6-oxo-1,6-dihydro-pyridine-3-carboxylic acid methyl ester was hydrolyzed following the procedure described in step 7 of example 9 to afford 73b.
step 3-{(S)-1-Phenyl-3-[5-(1,2,4-trimethyl-6-oxo-1,6-dihydro-pyridine-3-carbonyl)-hexahydro-pyrrolo[3,4-c]pyrrol-2-yl]-propyl}-carbamic acid tert-butyl ester (74a) was prepared by acylation of 48d with 73a as described in step 1 of example 12.
step 4—To a stirred solution of 74a (265 mg, 0.5 mmol) in DCM was added dropwise HCl (4M in 1,4-dioxane, 0.5 mL) dropwise. The reaction was stirred at RT for 90 minutes; the solvent was removed in vacuo and the residue was stripped with DCM. The crude amine 74b was used for next step without further purifications.
step 5 and 6-3-{(S)-1-Phenyl-3-[5-(1,2,4-trimethyl-6-oxo-1,6-dihydro-pyridine-3-carbonyl)-hexahydro-pyrrolo[3,4-c]pyrrol-2-yl]-propylcarbamoyl}-azetidine-1-carboxylic acid tert-butyl ester (75a) was prepared by acylation of 74b with azetidine-1,3-dicarboxylic acid mono-tert-butyl ester as described in step 5 of example 7. Deprotection of the BOC group was carried out with TFA/DCM as described in step 6 of example 24 to afford 75b.
step 7—To a solution of 75b (0.16 mmol) and TEA (83 mg) was added cyclopentanecarbonyl chloride (33 mg). The reaction was stirred for 2 h, poured into water and extracted 3 times with EtOAc. The combined organic extracts were dried (Na₂SO₄) filtered and evaporated. The crude residue was purified via SiO₂ chromatography eluting with DCM/MeOH/NH₄OH to afford 0.059 g of III-19.

Cyclopentanecarboxylic acid {(S)-1-phenyl-3-[-5-(1,2,4-trimethyl-6-oxo-1,6-dihydro-pyridine-3-carbonyl)-hexahydro-pyrrolo[3,4-c]pyrrol-2-yl]-propyl}-amide (III-13), 4,4-difluoro-cyclohexanecarboxylic acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide (III-15), 3,3-difluoro-cyclobutanecarboxylic acid

{(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide (III-16), tetrahydro-furan-3-carboxylic acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide (III-17) and 3-oxo-cyclobutanecarboxylic acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide (III-18) were prepared in analogous fashion except in step 5, azetidine-1,3-dicarboxylic acid mono-tert-butyl ester was replaced with cyclopentanecarboxylic acid, 4,4-difluoro-cyclohexane carboxylic acid, 3,3-difluoro-cyclobutane carboxylic acid, tetrahydrofuran-3-yl carboxylic acid and 3-oxo-cyclobutane carboxylic respectively, and steps 6 and 7 were omitted.

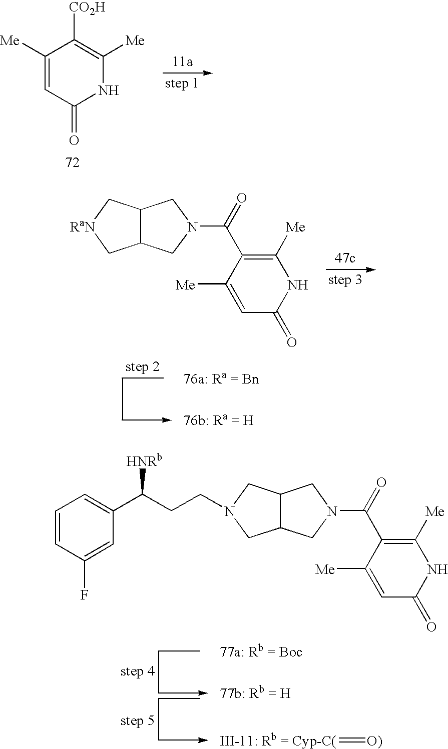

Cyclopentanecarboxylic acid [(S)-3-[5-(2,4-dimethyl-6-oxo-1,6-dihydro-pyridine-3-carbonyl)-hexahydro-pyrrolo[3,4-c]pyrrol-2-yl]-1-(3-fluoro-phenyl)-propyl]-amide (III-1) was prepared analogously except 72 was condensed with 2-benzyl-octahydro-pyrrolo[3,4-c]pyrrole (11a) using the procedure described in step 1 of example 12. Debenzylation (step 2) was carried out as described in step 7 of example 14. Reductive amination of 76b with 47c was carried out as described in step 5 of example 2. Removal of the BOC protecting group was carried out as described in step 4 of example 14 to afford 77b. Finally 77b was acylated with cyclopentane carbonyl chloride to afford III-11.

Cyclopentanecarboxylic acid {(S)-3-[5-(2,4-dimethyl-6-oxo-1,6-dihydro-pyridine-3-carbonyl)-hexahydro-pyrrolo[3,4-c]pyrrol-2-yl]-1-phenyl-propyl}-amide (III-3) was prepared similarly except ((S)-3-oxo-1-phenyl-propyl)-carbamic acid tert-butyl ester (CAS Reg No. 135865-78-0) was used in place of 47c.

4,4-Difluoro-cyclohexanecarboxylic acid [(S)-3-[5-(2,4-dimethyl-6-oxo-1,6-dihydro-pyridine-3-carbonyl)-hexahydro-pyrrolo[3,4-c]pyrrol-2-yl]-1-(3-fluoro-phenyl)-propyl]-amide and 3,3-difluoro-cyclobutanecarboxylic acid [(S)-3-[5-(2,4-dimethyl-6-oxo-1,6-dihydro-pyridine-3-carbonyl)-hexahydro-pyrrolo[3,4-c]pyrrol-2-yl]-1-(3-fluoro-phenyl)-propyl]-amide were prepared analogously except in step 5 cyclopentanecarbonyl chloride was replaced with 4,4-difluoro-cyclohexane carboxylic acid and 3,3-difluorocyclobutanecarboxylic acid and the coupling was mediated by TBTU as described step 1 of example 12 to afford III-12 and III-14, respectively.

### EXAMPLE 15

### Cyclopentanecarboxylic Acid {(S)-1-phenyl-3-[5-(1,4,6-trimethyl-2-oxo-1,2-dihydro-pyrimidine-5-carbonyl)-hexahydro-pyrrolo[3,4-c]pyrrol-2-yl]-propyl}-amide (III-20)

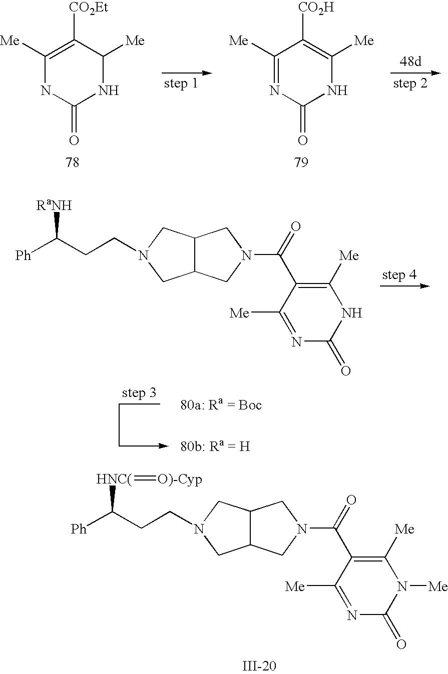

4,6-Dimethyl-2-oxo-1,2,3,4-tetrahydro-pyrimidine-5-carboxylic acid ethyl ester (78) was prepared as previously described (N.-Y. Fu et al., Tetrahedron 2002 58:4801-4807). 4,6-Dimethyl-2-oxo-1,2-dihydro-pyrimidine-5-carboxylic acid (79) was prepared from 78 as previously described (A. Pichala et al., J. Heterocyclic Chem. 2001 38:1345).

4,6-Dimethyl-2-oxo-1,2-dihydro-pyrimidine-5-carboxylic acid (79) was converted to III-20 by the procedure described in steps 3-7 of example 14.

### EXAMPLE 16

### 1-Acetyl-piperidine-4-carboxylic Acid (3-chloro-4-methyl-phenyl)-{3-[5-(2-pyrrolidin-1-yl-pyridine-3-sulfonyl)-hexahydro-pyrrolo[3,4-c]pyrrol-2-yl]-propyl}-amide Trifluoro Acetate (III-22)

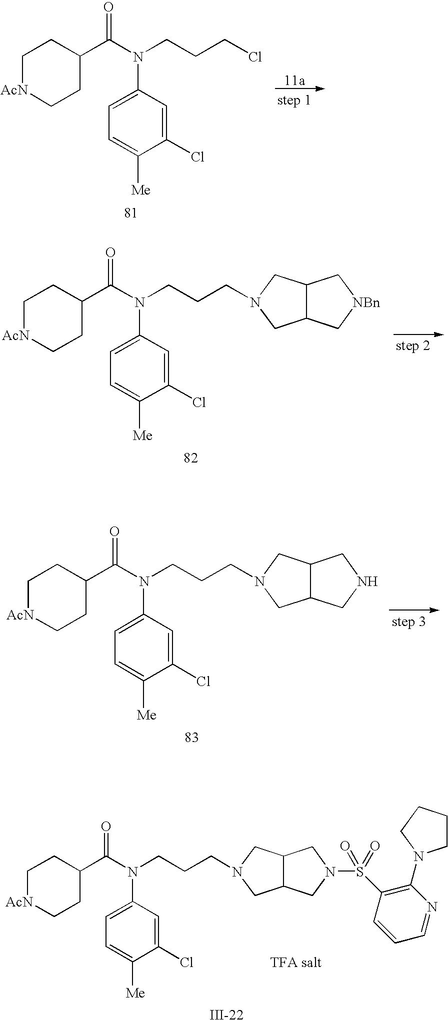

1-Acetyl-piperidine-4-carboxylic acid [3-(5-benzyl-hexahydro-pyrrolo[3,4-c]pyrrol-2-yl)-propyl]-(3-chloro-4-methyl-phenyl)-amide (82) was prepared by alkylation of 11a with 81 following the procedure described in step 4 of example 12. Debenzylation of 82 to afford 83 was carried put by the procedure described in step 2 of example 12.

step 3—To a solution of 2-chloro-pyridine-3-sulfonyl chloride (15.9 mg, 0.075 mmol) in DCM (1 mL) was added DIPEA (35 μL, 0.20 mmol) followed by 83 (0.05 mmol, 22 mg). The reaction was stirred at RT for 18 h then pyrrolidine (0.1 mL) was added and the mixture was stirred for an additional 18 hour. The reaction was filtered and the filtrate was concentrated. The residue was purified via preparative HPLC to afford III-22.

### EXAMPLE 17

### 1-Acetyl-piperidine-4-carboxylic Acid (3-chloro-4-methyl-phenyl)-{3-[5-(2-pyrrolidin-1-yl-pyridine-3-carbonyl)-hexahydro-pyrrolo[3,4-c]pyrrol-2-yl]-propyl}-amide Trifluoro-acetate (III-21)

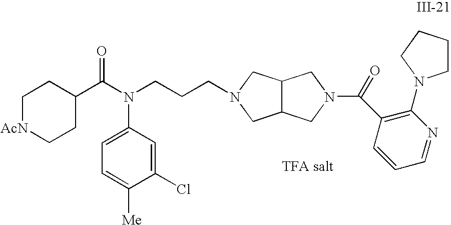

Oxalic chloride (0.3 mL) was added dropwise to a solution of 2-pyrrolidin-1-yl-nicotinic acid (14.4 mg) in MeCN (1 mL) with a drop of DMF. The reaction was stirred for 1 h at RT and the volatile material was evaporated. DIPEA (35 μL, 0.20 mmol) followed by a solution of 83 (0.05 mmol, 22 mg) in DCM (1 mL) was added to the residue. The reaction was stiffed for 18 h at RT, filtered and the filtrate was concentrated. The residue was purified by preparative HPLC to afford III-21.

### EXAMPLE 18

### 3-(5-{5-[(S)-3-(Cyclopentanecarbonyl-amino)-3-(R)-phenyl-propyl]-hexahydro-pyrrolo[3,4-c]pyrrole-2-carbonyl})-4,6-dimethyl-pyrimidin-2-yloxy)-2-methyl-propionic Acid Methyl Ester Trifluoro Acetate (I-11)

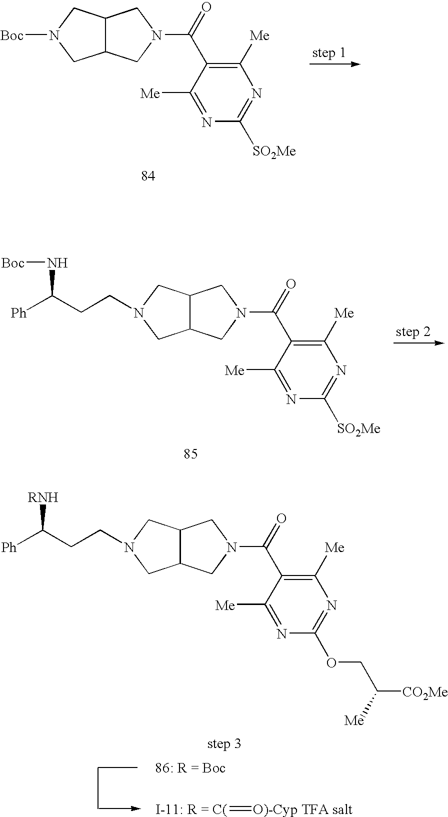 step 1—A solution of 84 (0.5 g, 1.2 mmol) in 1:1 TFA/DCM (5 mL) was stirred at RT for 1 h. The volatiles were evaporated and dried in vacuo. The residue was dissolved in DCM and ((S)-3-oxo-1-phenyl-propyl)-carbamic acid tert-butyl ester (0.27 g, 1.08 mmol) was added followed by NaBH(OAC)₃ (0.356 g, 1.68 mmol). The reaction mixture was stirred at RT overnight, diluted with EtOAc, washed with 10% aqueous NaHCO₃, and brine and dried (MgSO₄). The resulting EtOAc solution of 85 was filtered and evaporated and used directly in step 2.

step 2—A mixture of 85 (140 mg, 0.251 mmol), methyl (S)-(+)-3-hydroxy-2-methylpropionate (32 mg, 0.27 mmol), Cs₂CO₃ (0.2 g) in DMF (1.5 mL) was stirred at 50° C. overnight. The solvent was removed in vacuo and the residue was dissolved in EtOAc, washed twice with water and once with brine, dried (MgSO₄), filtered and evaporated to afford 0.100 g of 86 which was used in the next step without further purifications.

step 3—A solution of 86 (100 mg, 0.168 mmol) in 1:1 TFA/DCM (5 mL) was stirred at RT for 1 h. The volatiles were evaporated and dried in vacuo for 4 h at 40° C. The residue was dissolved in DCM and TEA (0.1 mL) and cyclopentanecarbonyl chloride (24 μL, 0.2 mmol) were added sequentially. The reaction mixture was stirred at RT overnight, concentrated and the residue was purified via preparative HPLC to afford I-11.

### EXAMPLE 19

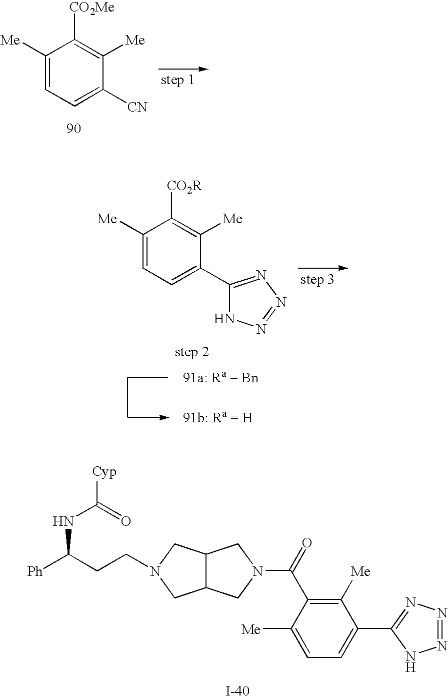 step 1—A modification of the procedure published by Wittenberger, et al. J. Org. Chem. 1993 58:4139-4141. A mixture of 90 (1.08 g, 5.71 mmol), azidotrimethylsilane (3.29 g, 28.53 mmol), dibutyltin oxide (256 mg, 1.03 mmol) and 12 mL of toluene was heated at 110° C. for 48 h. The reaction was cooled to RT and the volatiles were evaporated in vacuo. The residue was taken up in methanol and re-evaporated. The residue was partitioned between EtOAc and saturated NaHCO₃ and the resulting two phases filtered through CELITE® and the phases were separated. The organic phase was extracted twice with saturated NaHCO₃. The combined aqueous phases were acidified with 1M HCl and extracted twice with EtOAc. The combined organic phases were washed with water and brine, dried (Na₂SO₄), filtered and the filtrate was stripped in vacuo to afford 91a as a white crystalline solid: ms (ESI), m/z 233 (M+H).
step 2—A solution of propanethiol (970 mg, 12.74 mmol) and HMPA (10 mL) was cooled to 0° C. A solution of butyl lithium (4.3 mL, 6.88 mmol, 1.6M in hexanes) was added and the reaction was stirred at 0° C. for 10 min. A solution of 91a (256 mg, 1.10 mmol) and HMPA (2 mL) was added and the reaction was stirred at RT for 7 days. The resulting solution was quenched with water and partitioned between 1M HCl and ether. The aqueous phases were extracted with Et₂O. The combined organic phases were washed with water and brine, dried (Na₂SO₄), filtered and evaporated to a gold oil which crystallized upon standing. The crude material was taken up in refluxing EtOAc and filtered hot through a 4 μm filter to afford a clear yellow solution. The solution was evaporated in vacuo to afford 160 mg (67%) of 91b as a light yellow powder: ms (ESI), m/z 219 (M+H).
step 3—To a solution of 91b (100 mg, 0.46 mmol) and cyclopentanecarboxylic acid-[2-(hexahydropyrrolo-[3,4-c]pyrrol-2-yl)-1-phenylethyl]amide (120 mg, 0.35 mmol) in DMF (3 mL) was added a solution of (benzotriazol-1-yloxy)tripyrrolidinophosphonium hexafluorophosphate (239 mg, 0.46 mmol) and DMF (2 mL). DIPEA (305 mL, 1.75 mmol) was added and the reaction was stirred at RT for 4.5 h. The reaction was quenched with water, adjusted to pH 8 with 1M HCl and diluted with EtOAc. The aqueous phase was extracted with EtOAc. The aqueous layer was acidified to pH 2 with 1M HCl. and extracted twice with EtOAc and the combined organic phases were dried (Na₂SO₄), filtered and concentrated to a yellow oil. The crude material was purified by SiO₂ chromatography eluting with a gradient of DCM/MeOH (85:15) to DCM/MeOH/HOAc (85:15:2 drops), followed by preparative TLC developing with DCM/MeOH/HOAc (85:15:20 drops) to give I-45 as a mixture of rotational isomers. The product was dried under vacuum at 35° C. for 17 h to afford I-45 (24%) as a white powder: ms (ESI), m/z 542 (M+H).

### EXAMPLE 20

### Cyclopentanecarboxylic Acid {(S)-3-[5-(4,6-dimethyl-2-morpholin-4-yl-pyrimidine-5-carbonyl)-hexahydro-pyrrolo[3,4-c]pyrrol-2-yl]-1-phenyl-propyl}-amide (I-39)

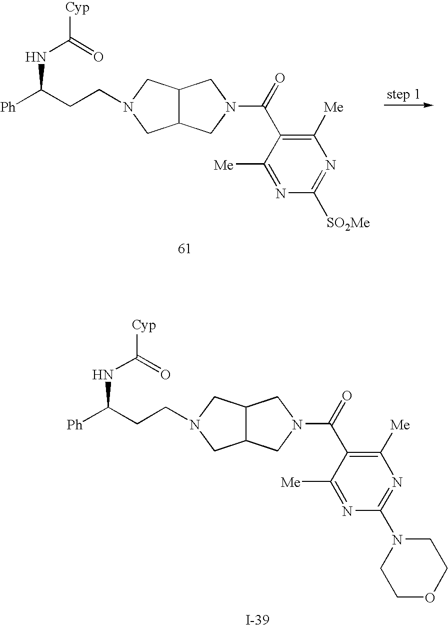

The methyl sulfone 61 (27 mg, 0.0488 mmol) and morpholine (13 μL, 0.146 mmol) were dissolved in THF (1 mL) and stirred at RT overnight. Water was added and the aqueous layer was extracted with EtOAc. The combined organic extracts were dried (Na₂SO₄), filtered and evaporated in vacuo. The residue was purified by preparative TLC and developed with a solution comprised of 30% 60:10:1 DCM/MeOH/NH₄OH and 70% DCM to afford 23 mg (66%) of 93 as white foam.

Cyclopentanecarboxylic acid ((S)-3-{5-[2-(4-acetyl-piperazin-1-yl)-4,6-dimethyl-pyrimidine-5-carbonyl]-hexahydro-pyrrolo[3,4-c]pyrrol-2-yl}-1-phenyl-propyl)-amide (I-40) was prepared similarly except morpholine was replaced with N-acetyl-piperazine.

Cyclopentanecarboxylic acid ((S)-3-{5-[4,6-dimethyl-2-(tetrahydro-pyran-4-ylamino)-pyrimidine-5-carbonyl]-hexahydro-pyrrolo[3,4-c]pyrrol-2-yl}-1-phenyl-propyl)-amide (I-41) was prepared similarly except morpholine was replaced with 4-amino-tetrahydrofuran.

### EXAMPLE 21

### Cyclopentanecarboxylic Acid {(S)-3-[5-(2-methanesulfonylamino-4,6-dimethyl-pyrimidine-5-carbonyl)-hexahydro-pyrrolo[3,4-c]pyrrol-2-yl]-1-phenyl-propyl}-amide (I-42)

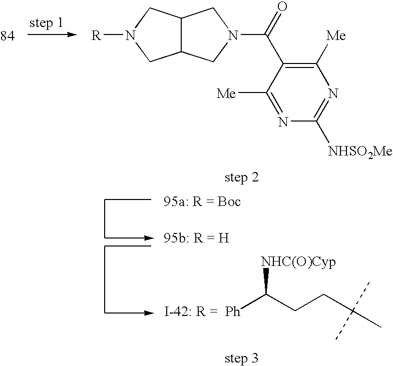

To a solution of 84 (296 mg, 0.7 mmol) and a minimal amount of DMSO was added NaH (61 mg) and the resulting solution was stirred for 15 min. Methane sulfonamide (132 mg) was added and stirred for 1 h. The reaction mixture was poured into dilute brine and extracted with 15% butanol in DCM. The volatile solvents were removed in vacuo and crude mixture was dried by lyophilization. The crude product was purified by SiO₂ chromatography eluting with gradient of 10% MeOH/0.5% NH₄OH/DCM and DCM gradient (80 to 20% DCM) to afford 207 mg of 95a and another 60 mg from mixed fractions containing mostly product.

Removal of the BOC protecting group and reductive amination with cyclopentanecarboxylic acid ((S)-3-oxo-1-phenyl-propyl)-amide was carried out as described in step 1 of example 18 to afford I-42.

### EXAMPLE 22

### 1-Acetyl-piperidine-4-carboxylic Acid (3-chloro-4-methyl-phenyl)-{3-[5-(2,4-dimethyl-6-oxo-1,6-dihydro-pyridine-3-carbonyl)-hexahydro-pyrrolo[3,4-c]pyrrol-2-yl]-butyl}-amide (102)

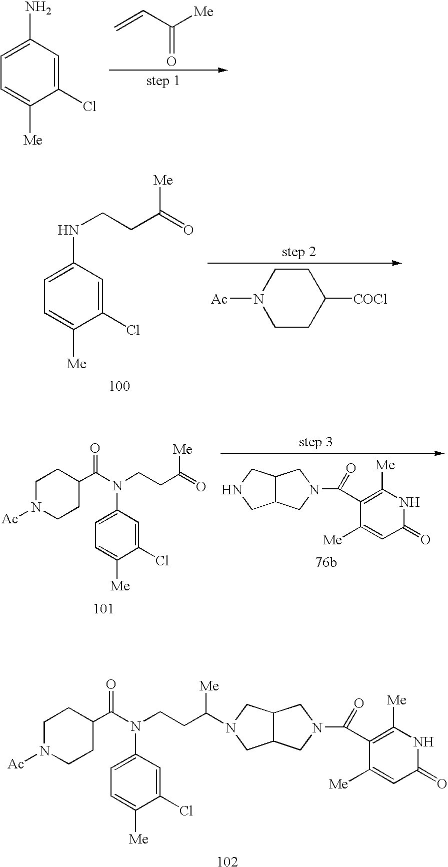 step 1—To a mixture of 3-chloro-4-methyl-phenylamine (2.5 g, 17.65 mmol) and trifluoromethanesulfonimide (0.33 g, 1.20 mmol) in MeCN (20 mL) was added methyl vinyl ketone (1 mL, 12.05 mmol) at RT. After 1 h silica gel and Na₂CO₃ (200 mg) were added to the mixture and it was concentrated in vacuo. The crude product was purified by SiO₂ column chromatography eluting with n-hexane:EtOAc (4:1) to afford 1.3 g (51%) of 100: NMR (CDCl₃) δ 2.15 (s, 3H), 2.25 (s, 3H), 2.73 (t, 2H), 3.35 (t, 2H), 3.93 (br, 1H), 6.4 (dd, 1H), 6.6 (d, 1H), 6.98 (d, 1H).
step 2—To a solution of 100 (1.3 g, 6.14 mmol) in DCM (30 mL) were added 1-acetyl-piperidine-4-carbonyl chloride (3.49 g, 18.42 mmol) and TEA (3 mL, 22.09 mmol) at 0° C. After 20 min the solution was heated at 40° C. overnight. The mixture was diluted with DCM and washed sequentially with H₂O, 2N HCl, saturated NaHCO₃ and brine. The organic layer was dried (Na₂SO₄), filtered and evaporated. The crude product was purified by SiO₂ column chromatography eluting with 5% MeOH/EtOAc to afford 1.28 g (57%) of 101: ¹H NMR (CDCl₃) δ 1.6-1.85 (m, 4H), 2.05 (s, 3H), 2.45 (s, 3H), 2.68 (t, 2H), 2.85 (t, 1H), 3.28 (d, 1H), 3.85-3.95 (m. 2H), 4.5 (d, 1H), 6.98 (dd, 1H), 7.2 (d, 1H), 7.33 (d, 1H).
step 3—To a solution of 101 (0.17 g, 0.48 mmol) in THE (7 mL) is added a solution of 76 (0.40 mmol) in DCM (7 mL). Titanium tetra-isopropoxide (0.26 mL, 0.89 mmol) is added to the mixture. The reaction is stirred for 40 min NaBH(OAc)₃ (0.13 g, 0.61 mmol) is added to the mixture and stirring is continued at RT overnight. Saturated NaHCO₃ is added to the mixture and it is stirred for 10 min. The mixture is filtered through a CELITE® pad and the filtrate is extracted with DCM. The organic layer is dried (MgSO₄) and is purified by SiO₂ column chromatography eluting with DCM:MeOH:NH₄OH (150:10:1) afford 102.

### EXAMPLE 23

### Cyclopropanecarboxylic Acid {(S)-3-[5-(2,4-dimethyl-6-oxo-1,6-dihydro-pyridine-3-carbonyl)-hexahydro-pyrrolo[3,4-c]pyrrol-2-yl]-2-methyl-1-phenyl-propyl}-amide (107)

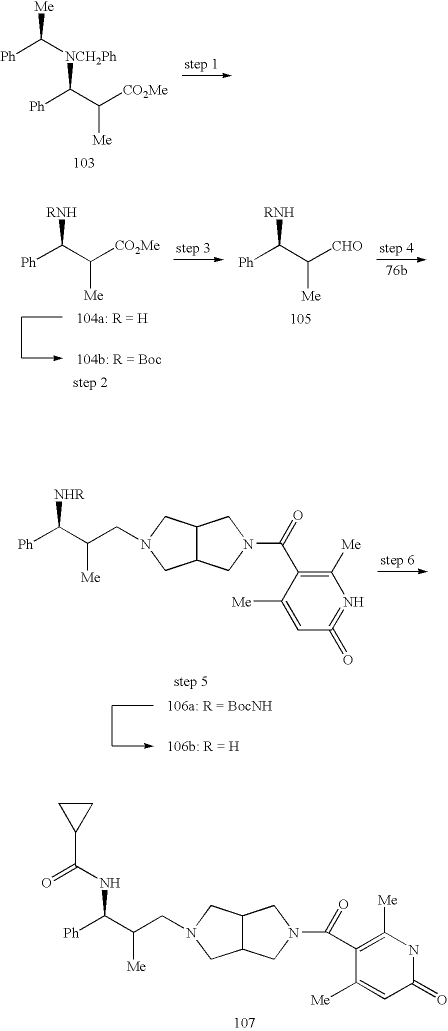 step 1—A solution of (2R,3S,αR)₃-[benzyl-(1-phenyl-ethyl)-amino]-2-methyl-3-phenyl-propionic acid methyl ester (103, 1.00 g, 2.58 mmol, prepared as described in J. Chem. Soc. Perkin Trans. 1 1994 1129) and MeOH:EtOAc: 10% HCl solution (25 mL) containing Pd(OH)₂—C (0.50 g) and hydrogenated (1 atm) for 24 h. The reaction mixture was filtered through a CELITE® pad to remove the catalyst. The filtrate was concentrated in vacuo and the residue partitioned between Et₂O (40 mL) and saturated NaHCO₃ solution (25 mL). The organic layer was dried (MgSO₄) and concentrated in vacuo to afford 408 mg (80%) of 104a as a pale yellow liquid: ms (ES+) m/z 194 (M+H)⁺.
step 2—A solution of (2R,3S)-3-amino-2-methyl-3-phenyl-propionic acid methyl ester (104a, 400 mg, 2.06 mmol) in THF (5 mL) was cooled to 0° C. A cold solution of NaOH (166 mg, 4.14 mmol) in H₂O (3.75 mL) was added to the above solution followed by a solution of (BOC)₂O in THF (2.5 mL) and the mixture stirred at RT for 5 h. The reaction mixture was extracted with EtOAc (2×50 mL) and the combined organic extracts were dried (MgSO₄) and concentrated in vacuo to afford 104b as a waxy solid: ms (ES+) m/z 237 (M-C₄H₈)⁺.
step 3—To a solution of 104b (355 mg, 1.21 mmol) in DCM (20 mL) cooled to −78° C. was added DIBAL-H (2.42 mL of 1 M DCM solution, 2.42 mmol) dropwise at such a rate to maintain the temperature below −70° C. After 2 h the reaction was quenched by the slow addition of MeOH (2 mL) and then allowed to warm to RT. The reaction mixture was filtered through a CELITE® pad. The filtrate was dried (Na₂SO₄) and concentrated in vacuo. The crude product was purified by flash chromatography on silica eluting with EtOAc:hexane (1:3) to afford 105 as a white solid: ¹H-NMR showed this material to be a 1:1.38 ratio of diastereomers.
step 4—To a solution of 105 (197 mg, 0.75 mmol) and 76b (0.75 mmol) in DCM (16 mL) containing HOAc (0.11 mL) is added NaBH(OAc)₃ (191 mg, 0.90 mmol) in 1 portion and the reaction is stirred for 18 h at RT. The reaction is quenched by the addition of 10% K₂CO₃ solution (10 mL) and is stirred for 20 min. The product is extracted with DCM (2×20 mL) and the combined extracts are dried (Na₂SO₄) and concentrated in vacuo. The crude product is purified by flash chromatography on silica eluting with DCM/7.5% MeOH (containing 2% NH₄OH) to afford 106a.
step 5—A solution of 106a (258 mg, 0.52 mmol) dissolved in 10 M HCl in MeOH (8 mL) is heated at 65° C. for 2 h. The MeOH is evaporated under reduced pressure and the residue is cautiously partitioned between DCM (25 mL) and 20% K₂CO₃ solution (15 mL). The aqueous layer is re-extracted with DCM (2×20 mL). The combined extracts are dried (Na₂SO₄) and concentrated in vacuo to afford 106b.
step 6—To a solution of 106b (0.050 mmol) and DIPEA (0.03 mL) is added cyclopropanecarbonyl chloride (6.8 μL, 7.8 mg, 0.075 mmol) and the resulting mixture is stirred at RT for 18 h. The reaction mixture is concentrated in a stream of N₂ and purified by reverse phase HPLC to afford 107.

### EXAMPLE 24

### 3,3-Difluoro-cyclobutanecarboxylic Acid ((S)-3-{5-[2-(1,1-dioxo-1λ⁶-isothiazolidin-2-yl)-4,6-dimethyl-pyrimidine-5-carbonyl]-hexahydro-pyrrolo[3,4-c]pyrrol-2-yl}-1-phenyl-propyl)-amide (I-49)

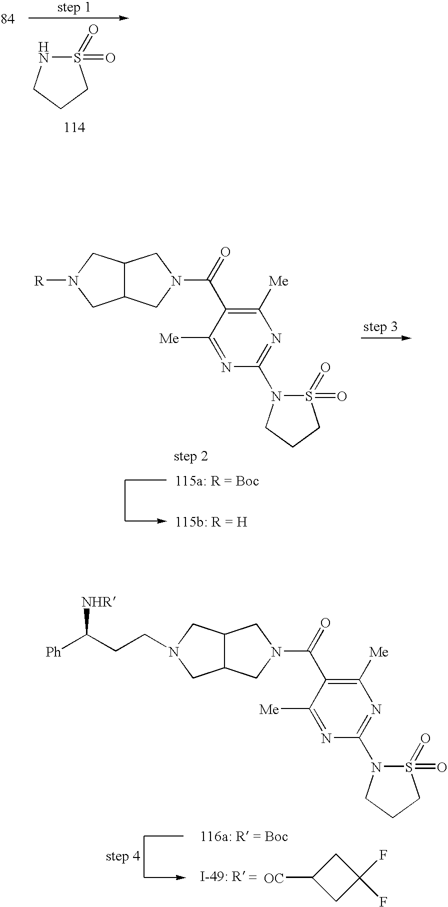 step 1—A solution of isothiazolidine 1,1-dioxide (114, 40 mg, 0.33 mmol; CAS Reg No. 5908-62-3) in THF (0.4 mL) and DMF (0.4 mL) was treated with NaH (14 mg, 60% dispersion in mineral oil) and heated to 80° C. for 5 min before a solution of 84 (116 mg, 0.27 mmol) in DMF (1.6 mL) was added. The reaction mixture was stirred at 80° C. for 5 min, allowed to cool to RT, quenched by the addition of water, extracted with EtOAc, dried (Na₂SO₄) and concentrated in vacuo. The residue was purified by SiO₂ column chromatography eluting with DCM:MeOH:NH₄OH (60/10/1) to afford 115 mg (90%) of 115a.

Removal of the BOC protecting group (step 2) and reductive amination (step 3) with ((S)-3-oxo-1-phenyl-propyl)-carbamic acid tert-butyl ester (117, CAS Reg. No. 143656-87-5) was carried out as described in step 1 of example 18 to afford 116a.

step 4—116a (15 mg, 0.025 mmol) was treated with HCl-dioxane and stirred at RT for 2 h. The solvent was removed and the residue was co-evaporated with Et₂O. To a suspension of the residue in DCM (1.5 mL) at 0° C. was added sequentially 3,3-difluoro-cyclobutanecarboxylic acid (6 mg, 0.044 mmol), HOBt (6 mg, 0.044 mmol), EDCl (7 mg, 0.037 mmol) and TEA (20 μL, 0.14 mmol). The reaction was stirred at RT for 22 h and quenched by addition of water. The mixture was extracted with DCM, the combined organic layers were dried (Na₂SO₄), filtered and concentrated. The residue was purified by SiO₂ column chromatography eluting with DCM:MeOH:NH₄OH (60/10/1) to afford 7 mg of I-49.

### EXAMPLE 25

### Cyclopentanecarboxylic Acid ((S)-3-{5-[2-(1-acetyl-pyrrolidin-3-ylamino)-4,6-dimethyl-pyrimidine-5-carbonyl]-hexahydro-pyrrolo[3,4-c]pyrrol-2-yl}-1-phenyl-propyl)-amide (I-46)

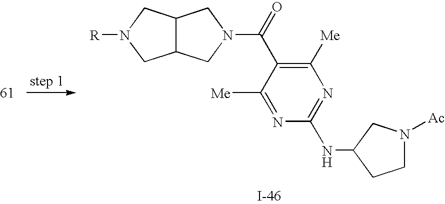 step 1—A solution of 61 (26 mg, 0.047 mmol) and 1-(3-amino-pyrrolidin-1-yl)-ethanone (18 mg, 0.14 mmol, cis/trans mixture) in THF (1 mL) was stirred at 70° C. The reaction mixture was purified by preparative TLC and developed with a solution comprised of 60% 60:10:1 DCM/MeOH/NH₄OH and 40% DCM to afford I-46.

### EXAMPLE 26

### Cyclopentanecarboxylic Acid ((S)-3-{5-[2-(3-hydroxy-pyrrolidin-1-yl)-4,6-dimethyl-pyrimidine-5-carbonyl]-hexahydro-pyrrolo[3,4-c]pyrrol-2-yl}-1-phenyl-propyl)-amide (I-47)

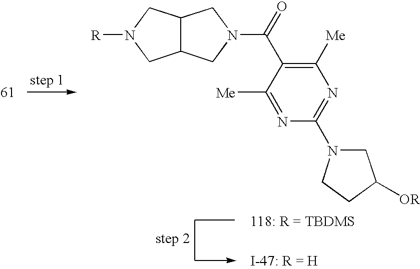

In analogous fashion to step 1 of example 26, 118 was prepared from the TBDMS-protected pyrrolidin-3-ol (cis/trans mixture; CAS Reg. No. 143656-87-5). Removal of the TBDMS protecting group with TFA/THF to afford I-47.

### EXAMPLE 27

### 3,3-Difluoro-cyclobutanecarboxylic Acid ((S)-3-{5-[2-(4-methanesulfonylamino-piperidin-1-yl)-4,6-dimethyl-pyrimidine-5-carbonyl]-hexahydro-pyrrolo[3,4-c]pyrrol-2-yl}-1-phenyl-propyl)-amide (I-51)

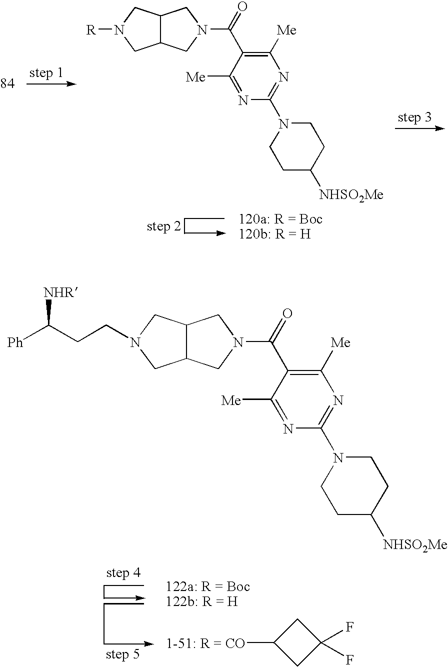

Condensation of 84 and N-piperidin-4-yl-methanesulfonamide (CAS Reg. No. 70724-72-0) was carried out as described in step 1 of example 25. Removal of the BOC protecting group (step 2) and reductive amination (step 3) with ((S)-3-oxo-1-phenyl-propyl)-carbamic acid tert-butyl ester (CAS Reg. No. 135865-78-0) to afford 122a was carried out as described in step 1 of example 18. After removal of the Boc protecting group of 122a (step 4) as described in step 1 of example 18, 122b was condensed with 3,3-difluoro-cyclobutanecarboxylic acid (step 5) as described in step 4 of example 24.

### EXAMPLE 28

### 3,3-Difluoro-cyclobutanecarboxylic Acid ((S)-3-{5-[2-(2-hydroxy-propylamino)-4,6-dimethyl-pyrimidine-5-carbonyl]-hexahydro-pyrrolo[3,4-c]pyrrol-2-yl}-1-phenyl-propyl)-amide (I-50)

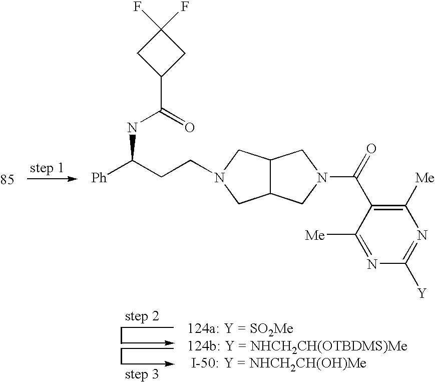 step 1—HCl-dioxane was added to 85 (231 mg, 0.4 mmol) and the mixture was stirred for 4 h. The solvent was evaporated. To a slurry of the residue in DCM at 0° C. was added TEA (0.23 mL, 1.6 mmol) and 3,3-difluoro-cyclobutanecarbonyl chloride (100 mg, 0.65 mmol), and the reaction mixture was stirred for 12 h. The solvent was removed and the residue was purified by SiO₂ column chromatography eluting with DCM:MeOH:NH₄OH (60:10:1) to afford 70 mg of 124a.

Condensation of TBDMS-protected 1-amino-propan-2-ol (CAS Reg. No. 791642-60-9) and 124a (step 2) was carried out at 45° C. as described in step 1 of example 25 to afford 124b. The TBDMS protecting group was removed (step 3) by treating 124b TFA./THF to afford I-50.

### EXAMPLE 29

### Tetrahydro-furan-3-carboxylic Acid [(S)-3-[5-(2-cyclopropyl-4,6-dimethyl-pyrimidine-5-carbonyl)-hexahydro-pyrrolo[3,4-c]pyrrol-2-yl]-1-(3-fluoro-phenyl)-propyl]-amide (I-48)

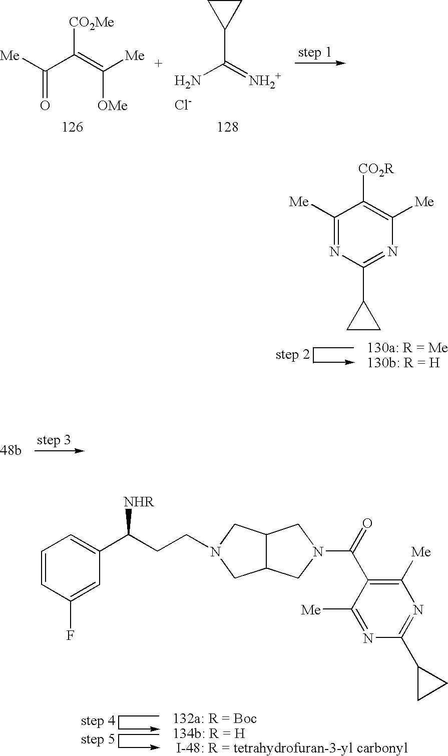 step 1—A solution of 126 (0.5 g, 2.904 mmol) and 128 (0.21 g, 1.742 mmol) in MeOH/acetone (1.5 mL/2 mL) was cooled down to 0° C. and a solution of potassium-tert-butoxide solution in tert-butanol (1.75 mL, 3.485 mmol) was added drop wise. After the addition was complete the mixture was slowly warmed to RT. After 4 h the solvent was evaporated. The residue was partitioned between DCM and water. A few drops of 1M HCl were added to bring the pH to ca.7. The layers were separated and the aqueous layer was extracted with DCM. The combined extracts were washed with brine, combined, dried (Na₂SO₄), filtered and evaporated. The residue was purified via SiO₂ chromatography which afforded a partially purified 130a, which was used in the following step without further purification.
step 2—The material obtained in step 1 (0.2 g, 0.97 mmol) was dissolved in EtOH (4 mL) and a solution of KOH (0.16 g, 2.905 mmol) and water (2 mL) was added. The solution stirred at 40° C. for 24 h. The solvent was completely evaporated. The residue was taken up in 1M hydrochloric acid (0.2 mL) and was extracted twice with EtOAc. The combined organic layers were dried (Na₂SO₄), filtered and evaporated to afford 130b as a white solid.
step 3—DIPEA (80 μL, 1.066 mmol) was added to a solution of 48b (0.130 g, 0.358 mmol), 130b (0.103 g, 0.536 mmol), EDCl (0.103 g, 0.537 mmol) and HOBt (0.072 g, 0.537 mmol) in DCM (2 mL). The resulting mixture was stirred at RT for 24 h then partitioned between water and DCM. The layers were separated and the aqueous layer was extracted twice with DCM. The combined organic layers were dried (Na₂SO₄), filtered and evaporated. The residue was purified via SiO₂ chromatography which afforded a partially purified 132a which was used directly in the following step.
step 4—TFA (83 μL) was added to a solution of 132a from step 1 in DCM (1.5 mL). The reaction was stirred at RT overnight, evaporated and dried over night under high vacuum to afford 0.153 g of 132b.
step 5—DIPEA (0.16 mL, 0.905 mmol) was added to a solution of 132b (0.05 g, 0.0906 mmol), tetrahydro-furan-3-carboxylic acid (16 μL, 0.136 mmol), EDCl (0.026 g, 0.136 mmol) and HOBt (0.018 g, 0.136 mmol) in DCM (1 mL). The resulting mixture was stirred at RT for 24 h then partitioned between water and DCM. The layers were separated and the aqueous layer was extracted twice with DCM. The combined organic layers were dried (Na₂SO₄), filtered and evaporated. The residue was purified via SiO₂ chromatography eluting with DCM/MeOH (95/5) and was further purified by HPLC to afford 0.010 g of I-48 as white foam.

### EXAMPLE 30

### Tetrahydro-furan-3-carboxylic Acid [(S)-3-[5-(6-ethynyl-2,4-dimethyl-pyridine-3-carbonyl)-hexahydro-pyrrolo[3,4-c]pyrrol-2-yl]-1-(3-fluoro-phenyl)-propyl]-amide (I-52)

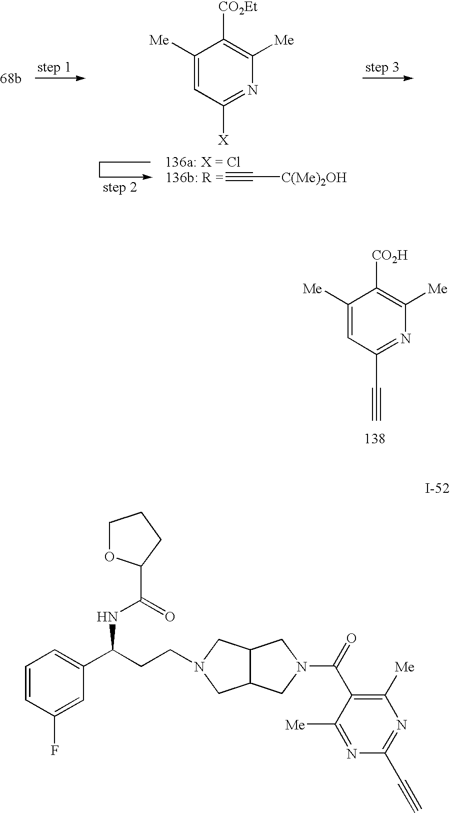 step 1—A mixture of 68b (5.0 g, 29.91 mmol) was suspended in POCl₃ (50 mL) and stirred at 85° C. degrees over night. The solvent was mostly evaporated. The residue was slowly poured into ice cold EtOH, stirred for 30 min and then partitioned between EtOAc and brine. The layers were separated and the aqueous layer was extracted EtOAc. The combined organic layers were dried (Na₂SO₄), filtered and evaporated. The residue was purified via SiO₂ chromatography which afforded 136a sufficiently pure to be used in the next step.
step 2—To a solution of 136a (1.0 g, 4.68 mmol) in DME (25 mL) was added water (11 mL), K₂CO₃ (1.98 g, 18.72 mmol), Cu(I)I (0.036 g, 0.187 mmol), P(Ph)₃ (0.098 g, 0.374 mmol) and 10% Pd/C (0.1 g, 93.6 μmol). The mixture stirred at RT for 30 minutes, then 2-methyl-3-butyn-2-ol (1.8 mL, 18.60 mmol) was added and the mixture stirred at 80° C. for 6 h then cooled to RT. The mixture was filtered through CELITE® and the filter cake washed with EtOAc. The combined organic layers were dried (Na₂SO₄), filtered and evaporated. The residue was purified via SiO₂ chromatography which afforded 136b which was used directly in the following step.
step 3—To a solution of 136b (0.458 g, 1.753 mmol) in EtOH (16 mL) was added a solution of KOH (0.29 g, 5.168 mmol) in water (4 mL) and the mixture stirred at reflux for 24 h. The solvent was evaporated and the residue was partitioned between water and EtAOc. The aqueous layer was acidified to pH 3 with con HCl and the resulting precipitate was filtered off and discarded. The aqueous layer was back extracted with EtOAc and the combined extracts were dried (Na₂SO₄), filtered and evaporated to give 0.058 g of 138.

The title compound (I-52) was prepared from 138 and 48b as described in steps 3-5 of Example 29

### EXAMPLE 31

### 3,3-Difluoro-cyclobutanecarboxylic Acid ((S)-3-{5-[2,4-dimethyl-6-oxo-1-(2,2,2-trifluoro-ethyl)-1,6-dihydro-pyridine-3-carbonyl]-hexahydro-pyrrolo[3,4-c]pyrrol-2-yl}-1-phenyl-propyl)-amide (III-23)

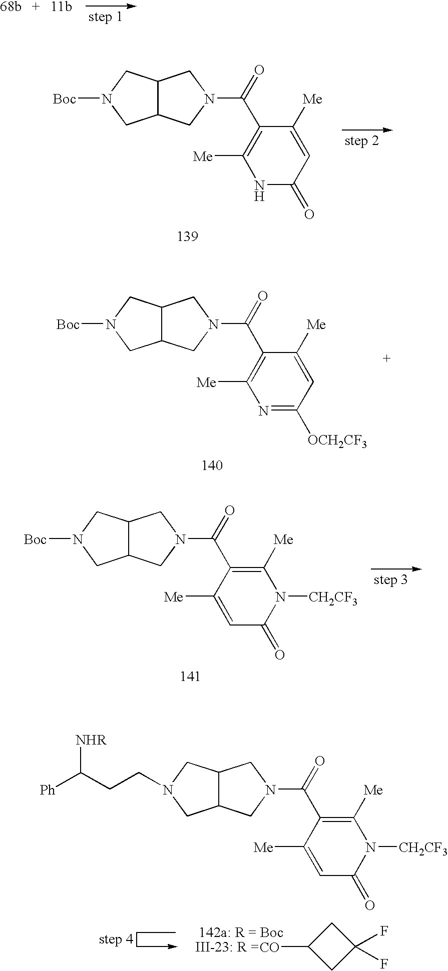 step 1—A solution of 68b (4.44 g), 2, (4.7 g), TEA (5.6 g), TBTU (9.25 g) in DCM and was stirred at RT overnight. The reaction mixture was poured into water and extracted with DCM, dried (Na₂SO₄), filtered and volatiles removed in vacuo. The crude product was purified by SiO₂ chromatography eluting with a MeOH/DCM gradient (0-6% MeOH containing 0.5% NH₄OH) to afford 5.73 g of 139.
step 2—To a solution of 139 (711 mg) in DME (4 mL) and DMF (1 mL) was added NaH (87 mg) and resulting mixture stirred for 15 min. Solid LiBr (342 mg) was then added and stirred for an additional 20 min after which 2,2,2-trifluoroethyl triflate (913 mg) was added and the reaction stirred overnight at 55° C. The reaction mixture was poured into water, extracted with DCM, and the combined extracts dried (Na₂SO₄), and adsorbed onto SiO₂. The adsorbed material was placed on top of a SiO₂ column and eluted with a MeOH/DCM gradient containing 0.5% NH₄OH (0-6.5% methanol) to afford 92 mg O-alkylated product 140 and 115 mg N-alkylated product 141 along with 380 mg of 139.
step 3—To a solution of 141 (115 mg) dissolved in a minimal amount of MeOH was added HCl in dioxane (3 mL of 4M solution) and the resulting mixture was stirred for 3.5 h at RT. The reaction mixture was concentrated in vacuo and the residue was dissolved in DCM. To this solution was added TEA (400 mg) and 117 (249 mg) and the resulting solution was stirred for 15 min then NaBH(OAc)₃ (88 mg) was added and stirring continued for 3 h at RT. The solution was poured into water/brine/NaHCO₃ and extracted with DCM. The combined DCM extracts were dried (Na₂SO₄), filtered and evaporated. The crude product was purified by SiO₂ chromatography eluting with MeOH/DCM gradient containing 0.5% NH₄OH (0 to 7% MeOH) to afford 130 mg of 142.
step 4—To a solution of 142 (130 mg) dissolved in a minimal amount of MeOH was added a solution of HCl in dioxane (3 mL of 4M HCl) and the resulting solution stirred at RT for 3 h. The reaction was concentrated in vacuo and the residue dissolved in DCE (10 mL) and TEA (100 mg), 3,3-difluoro-cyclobutane-carboxylic acid (40 mg) and TBTU (94 mg). The reaction mixture was stirred overnight at RT, poured into water/brine and extracted with DCM. The combined extracts were dried (Na₂SO₄), filtered and evaporated. The crude product was purified by SiO2 chromatography eluting with a MeOH/DCM gradient containing 0.5% NH₄OH (0 to 7% MeOH) to afford 112 mg of III-23.

### EXAMPLE 32

### 3,3-Difluoro-cyclobutanecarboxylic Acid ((S)-3-{5-[2,4-dimethyl-6-(2-oxo-propoxy)-pyridine-3-carbonyl]-hexahydro-pyrrolo[3,4-c]pyrrol-2-yl}-1-phenyl-propyl)-amide (III-26) and cyclopentanecarboxylic Acid ((S)-3-{5-[2,4-dimethyl-6-oxo-1-(2-oxo-propyl)-1,6-dihydro-pyridine-3-carbonyl]-hexahydro-pyrrolo[3,4-c]pyrrol-2-yl}-1-phenyl-propyl)-amide (III-24)

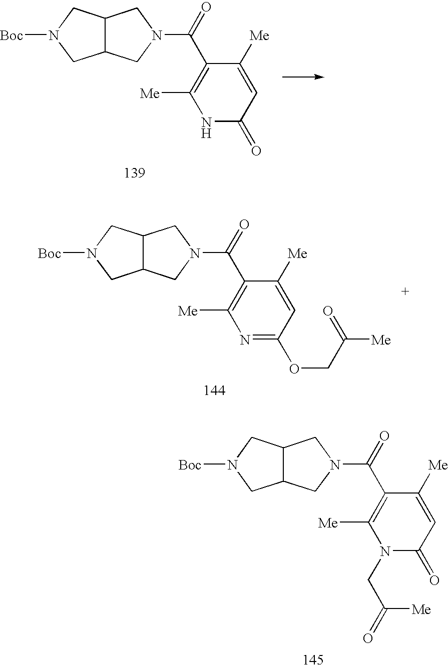

A solution of 139 (180 mg), chloro acetone (14 mg) and Cs₂CO₃ (325 mg) in MeCN was stirred at RT overnight, poured into water/brine and extracted with DCM. The combined extracts were dried (Na2SO4), filtered and evaporated and the crude product purified by SiO2 chromatography eluting with a MeOH/DCM gradient containing 0.5% NH₄OH to afford 96 mg of 144 and 70 mg of 145 (86% yield of N- and O-alkylated product, 4:50-alkylated).

When the reaction was run in similar fashion except Cs₂CO₃/MeCN was replaced with NaH/DME/DMF, a 75% combined yield was obtained but the N-alkylated compound was the predominant product (4:1)

III-26 was prepared from 144 by the procedure described in steps 3 and 4 of example 31. III-24 was prepared from 145 by the procedure described in steps 3 and 4 of example 31 except in step 4 was carried out with cyclopentane carbonyl chloride as follows:

The deprotected product was dissolved in DCM and TEA (400 mg) wg was added followed by cyclopentane carbonyl chloride (76 mg) and the resulting mixture stirred overnight at RT. The reaction mixture was poured into water/brine and extracted with DCM. The combined extracts were dried (Na₂SO₄), filtered and evaporated and the crude product purified by SiO2 chromatography eluting with a MeOH/DCM gradient containing 0.5% NH₄OH (0 to 6% MeOH) to afford 176 mg of III-26.

### EXAMPLE 33

### Cyclopentanecarboxylic Acid ((S)-3-{5-[1-(2-hydroxy-propyl)-2,4-dimethyl-6-oxo-1,6-dihydro-pyridine-3-carbonyl]-hexahydro-pyrrolo[3,4-c]pyrrol-2-yl}-1-phenyl-propyl)-amide (III-25)

To a solution of III-26 (131 mg) in MeOH was added NaBH₄ (18 mg) and the resulting mixture was stirred overnight at RT. The reaction mixture was poured into water/brine and extracted with DCM. The combined extracts were dried, filtered and evaporated. The crude product was purified by SiO2 chromatography eluting with a MeOH/DCM gradient containing 0.5% NH₄OH (0 to 6.5% MeOH) to afford 116 mg of III-25.

### EXAMPLE 34

### 3-Hydroxy-cyclobutanecarboxylic Acid ((S)-3-{5-[6-(2-hydroxy-propoxy)-2,4-dimethyl-pyridine-3-carbonyl]-hexahydro-pyrrolo[3,4-c]pyrrol-2-yl}-1-phenyl-propyl)-amide (III-27)

The title compound was prepared from 144 by the procedure described in steps 3 and 4 of example 31 except in step 4 fluorocyclobutane-carboxylic acid was replace by 3-oxo-cyclobutane-carboxylic acid. Reduction of the product with NaBH₄ as described in example 33 affords (III-27)

### EXAMPLE 35

### Cyclopentanecarboxylic acid {(S)-3-[5-(5-chloro-2,4-dimethyl-6-oxo-1,6-dihydro-pyridine-3-carbonyl)-hexahydro-pyrrolo[3,4-c]pyrrol-2-yl]-1-phenyl-propyl}-amide (III-28)

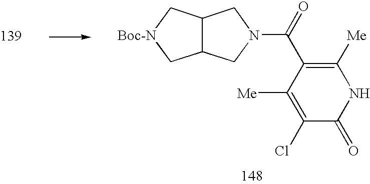

To a solution of 3 (1.083 g) in HOAc/DCM was added NCS (521 mg) and the resulting mixture stirred at RT overnight. An additional aliquot of NCS (200 mg) and stirring was continued at 55° C. over the weekend. The reaction mixture was poured into water/brine and extracted with DCM. The combined extracts were dried (Na₂SO₄), filtered and evaporated. The crude product was purified by SiO₂ chromatography eluting with a MeOH/DCM gradient containing 0.5% NH₄OH to afford 290 mg of 148.

Removal of the BOC protecting group and reductive amination with ((S)-3-oxo-1-phenyl-propyl)-carbamic acid tert-butyl ester (117, CAS Reg. No. 143656-87-5) was carried out as described in step 1 of example 18. The Boc protecting group on the side chain is removed with TFA/THF as described previously and acylation of the resulting amine with cyclopentane carbony chloride is carried out as described in Example 32.

### EXAMPLE 36

### CCR5-Mediated CCF Assay

CCF assay was performed as described before (C. Ji, J. Zhang, N. Cammack and S. Sankuratri 2006 J. Biomol. Screen. in press). Hela-R5 cells (express gp160 from R5-tropic virus and HIV-1 T at) were plated in 384 well white culture plates (BD Bioscience, Palo Alto, Calif.) at 7.5×10³ cells per well in phenol red-free Dulbecco's Modified Eagle Medium (DMEM) supplemented with 10% FBS, 1× Pen-Strep, 300 μg/ml G418, 100 μg/ml hygromycin, and 1 μg/ml doxycycline (Dox) (BD Bioscience, Palo Alto, Calif.), using Multimek (Beckman, Fullerton, Calif.) and incubated at 37° C. overnight to induce the expression of gp160. Ten μl diluted compounds in medium containing 5% DMSO were added to the cells, followed by the addition of CEM-NKr-CCR5-Luc (obtained from NIH AIDS Research & Reference Reagents Program) that expresses CD4 and CCR5 and carries a HIV-2 long terminal repeat (LTR)-driven luciferase reporter gene at 1.5×10⁴ cells/15 μl/well and incubated for 24 hrs. At the end of co-culture, 15 μl of Steady-Glo luciferase substrate was added into each well, and the cultures were sealed and gently shaken for 45 min. The luciferase activity were measured for 10 sec per well as luminescence by using 16-channel TopCount NXT (PerkinElmer, Shelton, Conn.) with 10 min dark adaptation and the readout is count per second (CPS). For the drug interaction experiments, small molecule compounds or antibodies were serially diluted in serum-free and phenol red-free RPMI containing 5% dimethyl sulfoxide (DMSO) (CalBiochem, La Jolla, Calif.) and 1× Pen-Strep. Five μl each of the two diluted compound or mAb to be tested for drug-drug interactions were added to the Hela-R5 cells right before the addition of target cells. The checker board drug combinations at various concentrations were carried out as shown in FIG. 1A.

TABLE 4

| Cpd. No. | IC₅₀ (μM) |
|---|---|
| I-7 | <0.0003 |
| I-9 | <0.0003 |
| I-42 | <0.0003 |
| II-3 | <0.0003 |
| I-19 | 0.0004 |
| I-37 | 0.0004 |
| I-3 | 0.0005 |
| I-8 | 0.0009 |
| I-16 | 0.001 |
| I-10 | 0.0011 |
| III-15 | 0.0011 |
| III-13 | 0.0014 |

### EXAMPLE 37

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. | |
|---|---|---|
| Active ingredient | 0.25 | g |
| Sodium Chloride | q.s. to make isotonic | |
| Water for injection to | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound according to formula I wherein: 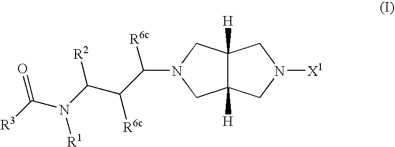
one of R¹ and R² is phenyl optionally substituted with one to four substitutents selected independently in each incidence from the group consisting of halogen, C₁₋₆ alkyl and C₁₋₆ alkoxy; and, the other of R¹ and R² is hydrogen;
R³ is selected from the group consisting of:
(a) C₃₋₇ cycloalkyl optionally substituted with 1 to 4 fluorines, cyano, hydroxyl, C₁₋₃ alkyl or phenyl, 4-oxo-cyclohexyl or 3-oxo-cyclobutyl;
(b) heterocycle selected from the group consisting of IIa-c, oxetanyl and tetrahydrofuranyl, 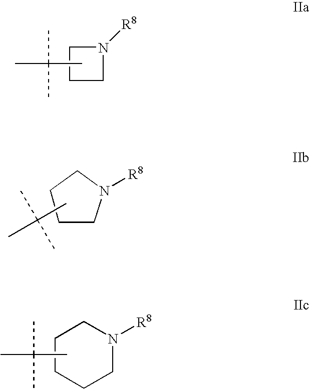
wherein:
R⁸ is hydrogen, COR⁹, COCHR¹⁴NHR¹⁵ or SO₂R¹⁰; and,
R⁹ is C₁₋₆ alkyl or C₃₋₇ cycloalkyl;
R¹⁰ is C₁₋₆ alkyl;
R¹⁴ is the side chain of naturally occurring amino acid;
R¹⁵ is hydrogen, tert-butoxycarbonyl or benzyloxycarbonyl;
(c) C₁₋₆ alkyl; and
(d) C₁₋₆ alkoxy;
R⁶ is hydrogen, C₁₋₃ alkyl, C₁₋₃ haloalkyl, C₁₋₆ hydroxyalkyl or oxo-C₁₋₆ alkyl;
R^{6a}, R^{6b}, R^{6c} and R^{6d} are independently hydrogen or C₁₋₃ alkyl with the proviso that at least one of R^{6c} is hydrogen;
X¹ is selected from the group consisting of (i)-(ix) and (x):
wherein
X² is N or CH;
X^{2a} is N, CH or CCl;
A¹ is phenylene or C₁₋₆ straight or branched alkylene optionally substituted by a phenyl ring;
R⁵ is hydroxy, C₁₋₆ alkoxy, benzyloxy or NR^{6a}R^{6b}; 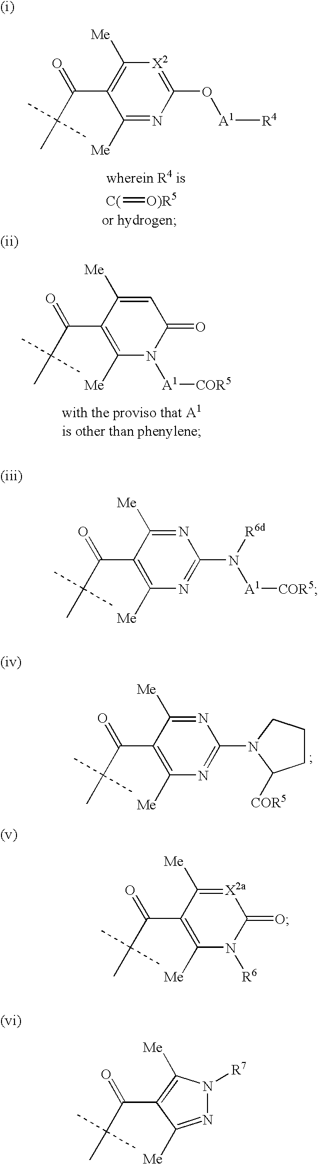
wherein:
R⁷ is C₃₋₇ cycloalkyl, (CH₂)ₙCOR⁵, heteroaryl selected from the group consisting of pyridine, pyrimidine, pyrazine and pyridazine said heteroaryl optionally substituted with C₁₋₃ alkyl or C₁₋₃ haloalkyl;
n is 1 to 3; 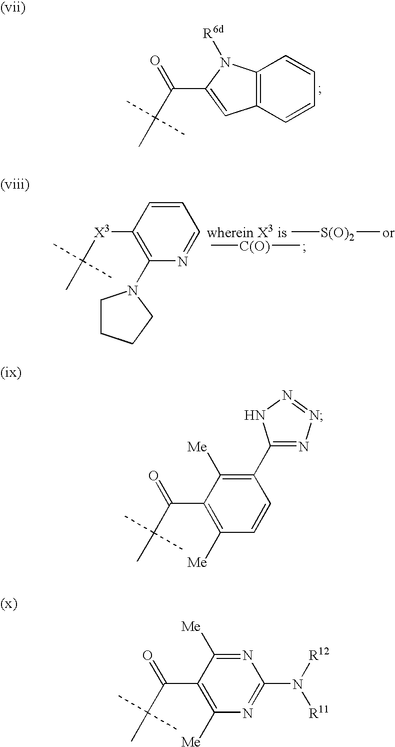
wherein
R¹¹ and R¹² are (A) together a group (CH₂)₂X⁴(CH₂)₂, (CH₂)₂CH(R¹⁶)CH₂, or (CH₂)₂SO₂; or, (B) independently R¹² is hydrogen or C₁₋₃ alkyl and R¹¹ is —SO₂C₁₋₆ alkyl, C₁₋₆ hydroxyalkyl, xA, xB or xC; 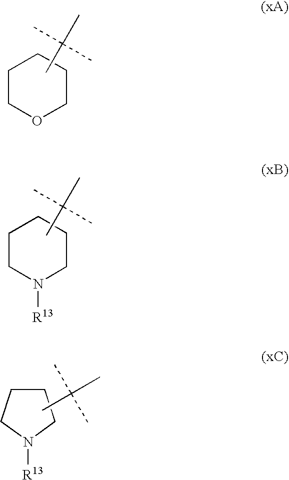
X⁴ is O, S(O)ₘ, NR¹³ or CH(NHSO₂C₁₋₆ alkyl);
R¹³ is R^{6d}, —C(O)C₁₋₆ alkyl, S(O)₂C₁₋₆ alkyl;
R¹⁶ is hydrogen, hydroxyl or C₁₋₁₀ acyloxy;
m is zero to two; and, 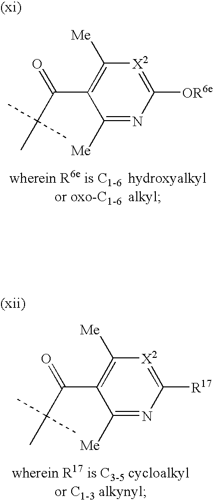
or pharmaceutically acceptable salts, hydrates or solvates thereof.

2. A compound according to claim 1 wherein:
R⁶ is hydrogen or C₁₋₃ alkyl;
X^{2a} is N or CH;
X¹ is selected from the group consisting of (i) to (ix) and (x) and when X¹ is (x), R¹¹ and R¹² are (A) together a group (CH₂)₂X⁴(CH₂)₂ or (B) independently R¹² is hydrogen or C₁₋₃ alkyl and R¹¹ is —SO₂C₁₋₆ alkyl, xA, xB wherein X⁴ is O, S(O)ₘ or NR¹³.

3. A compound according to claim 1 wherein:
R¹ is hydrogen;
R² is phenyl optionally substituted with fluoro or chloro;
R³ is selected from the group consisting of:
(a) C₃₋₇ cycloalkyl optionally substituted with one to four fluorines, 4-oxo-cyclohexyl or 3-oxo-cyclobutyl;
(b) heterocycle selected from the group consisting of IIa, IIc and tetrahydrofuranyl,
wherein:
R⁸ is COR⁹ and,
R⁹ is C₁₋₆ alkyl or C₃₋₇ cycloalkyl;
(c) C₁₋₆ alkyl; and
(d) C₁₋₆ alkoxy;
R^{6c} in each occurrence is hydrogen;
X¹ is selected from the group consisting of (i), (iii), (v) and (vi) wherein:
A¹ is C₁₋₆ straight or branched alkylene;
R⁷ is C₃₋₇ cycloalkyl or heteroaryl selected from the group consisting of pyridine, pyrimidine, pyrazine and pyridazine said heteroaryl optionally substituted with C₁₋₃ alkyl or C₁₋₃ haloalkyl; and,
X^{2a} is N or CH.

4. A compound according to claim 3 wherein:
R³ is (a) C₃₋₇ cycloalkyl optionally substituted with one to four fluorines, 4-oxo-cyclohexyl or 3-oxo-cyclobutyl; or,
(b) heterocycle selected from the group consisting of 1a or tetrahydrofuranyl,
wherein:
R⁸ is COR⁹ and,
R⁹ is C₁₋₆ alkyl or C₃₋₇ cycloalkyl;
X¹ is (vi) wherein R⁷ is heteroaryl selected from the group consisting of pyridine, pyrimidine, pyrazine and pyridazine said heteroaryl optionally substituted with C₁₋₃ alkyl or C₁₋₃ haloalkyl.

5. A compound according to claim 3 wherein:
R³ is selected from the group consisting of:
(a) C₃₋₇ cycloalkyl optionally substituted with one to four fluorines, 4-oxo-cyclohexyl or 3-oxo-cyclobutyl;
(b) heterocycle selected from the group consisting of 1a or tetrahydrofuranyl,
wherein R⁸ is COR⁹ and R⁹ is C₁₋₆ alkyl;
X¹ is (v) and R⁶ is C₁₋₆ alkyl; and,
X^{2a} is N or CH.

6. A compound according to claim 3 wherein:
R³ is C₃₋₇ cycloalkyl optionally substituted with one to four fluorines, 4-oxo-cyclohexyl or 3-oxo-cyclobutyl;
X¹ is (i) or (iii) and R⁵ is hydroxy or C₁₋₆ alkoxy.

7. A compound according to claim 1 wherein:
R¹ is phenyl optionally substituted with one to four substitutents selected independently in each incidence from the group consisting of halogen and C₁₋₆ alkyl;
R² is hydrogen;
R³ is selected from the group consisting of:
(a) C₃₋₇ cycloalkyl optionally substituted with one to four fluorines, 4-oxo-cyclohexyl or 3-oxo-cyclobutyl;
(b) heterocycle selected from the group consisting of IIa, IIc, oxetanyl and tetrahydrofuranyl,
wherein:
R⁸ is COR⁹ and,
R⁹ is C₁₋₆ alkyl or C₃₋₇ cycloalkyl;
(c) C₁₋₆ alkyl; and,
(d) C₁₋₆ alkoxy;
R^{6c} in each occurrence is hydrogen;
X¹ is selected from the group consisting of (i), (iii), (v) and (vi) wherein:
A¹ is C₁₋₆ straight or branched alkylene;
R⁷ is C₃₋₇ cycloalkyl or heteroaryl selected from the group consisting of pyridine, pyrimidine, pyrazine and pyridazine said heteroaryl optionally substituted with C₁₋₃ alkyl or C₁₋₃ haloalkyl; and,
X² is N or CH.

8. A compound according to claim 7 wherein R³ is C₃₋₇ cycloalkyl optionally substituted with 1 to 4 fluorines or IIc, R⁸ is COR⁹, R⁹ is C₁₋₆ alkyl, X¹ is (i) or (v), X² is CH, and R⁵ is hydroxy or C₁₋₆ alkoxy.

9. A compound according to claim 7 wherein R³ is C₃₋₇ cycloalkyl optionally substituted with 1 to 4 fluorines or IIc, R⁸ is COR⁹, R⁹ is C₁₋₆ alkyl, X¹ is (iii) and R⁵ is hydroxy or C₁₋₆ alkoxy.

10. A compound according to claim 1 wherein R¹ is hydrogen, R² is optionally substituted phenyl, R³ is C₃₋₇ cycloalkyl optionally substituted with 1 to 4 fluorines, 4-oxo-cyclohexyl or 3-oxo-cyclobutyl, X¹ is (x), R¹¹ is SO₂C₁₋₆ alkyl and R¹² is hydrogen or C₁₋₃ alkyl.

11. A compound according to claim 1 herein R¹ is hydrogen, R² is optionally substituted phenyl, R³ is C₃₋₇ cycloalkyl optionally substituted with 1 to 4 fluorines, 4-oxo-cyclohexyl or 3-oxo-cyclobutyl, X¹ is (x), and (A) R¹¹ and R¹² together are (CH₂)₂X⁴(CH₂)₂, X⁴ is O or NR¹³ and R¹³ is C(O)C₁₋₆ alkyl or (B) R¹¹ is 4-tetrahydropyran-4-yl and R¹² is hydrogen.

12. A compound according to claim 1, or a pharmaceutical acceptable salt thereof, selected from the group consisting of:
cyclopentanecarboxylic acid {(S)-3-[5-(4,6-dimethyl-2-methylcarbamoylmethoxy-pyrimidine-5-carbonyl)-hexahydro-pyrrolo[3,4-c]pyrrol-2-yl]-1-phenyl-propyl}-amide;
cyclopentanecarboxylic acid {(S)-3-[5-(2-carbamoylmethoxy-4,6-dimethyl-pyrimidine-5-carbonyl)-hexahydro-pyrrolo[3,4-c]pyrrol-2-yl]-1-phenyl-propyl}-amide;
(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-acetic acid benzyl ester;
2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid methyl ester;
(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-ylamino)-acetic acid; TFA salt;
2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid; TFA salt;
4-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-benzoic acid benzyl ester;
4-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-benzoic acid; TFA salt;
3-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-benzoic acid ethyl ester;
3-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-benzoic acid, HCl salt;
3-(5-{5-[(S)-3-(cyclopentanecarbonyl-amino)-3-(R)-phenyl-propyl]-hexahydro-pyrrolo[3,4-c]pyrrole-2-carbonyl}-4,6-dimethyl-pyrimidin-2-yloxy)-2-methyl-propionic acid methyl ester; TFA salt;
(R)-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-phenyl-acetic acid methyl ester;
(S)-2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid methyl ester;
2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-butyric acid;
(S)-2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid;
(S)-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-phenyl-acetic acid methyl ester;
(S)-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-phenyl-acetic acid;
(R)-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-phenyl-acetic acid;
cyclopentanecarboxylic acid {(S)-3-[5-(2-methoxy-4,6-dimethyl-pyrimidine-5-carbonyl)-hexahydro-pyrrolo[3,4-c]pyrrol-2-yl]-1-phenyl-propyl}-amide;
(R)-2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid;
2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-butyric acid ethyl ester;
(R)-2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-propionic acid ethyl ester;
(S)-1-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yl)-pyrrolidine-2-carboxylic acid ethyl ester;
(S)-1-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yl)-pyrrolidine-2-carboxylic acid;
(S)-1-[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-yl]-pyrrolidine-2-carboxylic acid ethyl ester;
2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-2-methyl-propionic acid methyl ester;
(S)-2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-ylamino)-3-methyl-butyric acid ethyl ester;
(S)-2-[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-ylamino]-3-methyl-butyric acid ethyl ester;
(S)-1-[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-yl]-pyrrolidine-2-carboxylic acid;
(S)-2-[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-ylamino]-3-methyl-butyric acid;
(S)-2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-ylamino)-3-methyl-butyric acid;
2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-yloxy)-2-methyl-propionic acid;
(S)-2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-ylamino)-propionic acid ethyl ester;
(R)-2-[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-ylamino]-propionic acid ethyl ester;
(R)-2-(5-{5-[(S)-3-(cyclopentanecarbonyl-amino)-3-phenyl-propyl]-hexahydro-pyrrolo[3,4-c]pyrrole-2-carbonyl}-4,6-dimethyl-pyrimidin-2-ylamino)-propionic acid;
(R)-2-[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-ylamino]-propionic acid;
{[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-yl]-methyl-amino}-acetic acid ethyl ester;
{[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-pyrimidin-2-yl]-methyl-amino}-acetic acid;
cyclopentanecarboxylic acid {(S)-3-[5-(4,6-dimethyl-2-morpholin-4-yl-pyrimidine-5-carbonyl)-hexahydro-pyrrolo[3,4-c]pyrrol-2-yl]-1-phenyl-propyl}-amide;
cyclopentanecarboxylic acid (3-{5-[2-(4-acetyl-piperazin-1-yl)-4,6-dimethyl-pyrimidine-5-carbonyl]-hexahydro-pyrrolo[3,4-c]pyrrol-2-yl}-1-phenyl-propyl)-amide;
cyclopentanecarboxylic acid ((S)-3-{5-[4,6-dimethyl-2-(tetrahydro-pyran-4-ylamino)-pyrimidine-5-carbonyl]-hexahydro-pyrrolo[3,4-c]pyrrol-2-yl}-1-phenyl-propyl)-amide;
cyclopentanecarboxylic acid {(S)-3-[5-(2-methanesulfonylamino-4,6-dimethyl-pyrimidine-5-carbonyl)-hexahydro-pyrrolo[3,4-c]pyrrol-2-yl]-1-phenyl-propyl}-amide;
cyclopentanecarboxylic acid ((S)-3-{5-[2-(methanesulfonyl-methyl-amino)-4,6-dimethyl-pyrimidine-5-carbonyl]-hexahydro-pyrrolo[3,4-c]pyrrol-2-yl}-1-phenyl-propyl)-amide;
cyclopentanecarboxylic acid {(S)-3-[5-(1-methyl-1H-indole-2-carbonyl)-hexahydro-pyrrolo[3,4-c]pyrrol-2-yl]-1-phenyl-propyl}-amide;
cyclopentanecarboxylic acid ((S)-3-{5-[2,6-dimethyl-3-(1H-tetrazol-5-yl)-benzoyl]-hexahydro-pyrrolo[3,4-c]pyrrol-2-yl}-1-phenyl-propyl)-amide;
cyclopentanecarboxylic acid ((S)-3-{5-[2-(1-acetyl-pyrrolidin-3-ylamino)-4,6-dimethyl-pyrimidine-5-carbonyl]-hexahydro-pyrrolo[3,4-c]pyrrol-2-yl}-1-phenyl-propyl)-amide;
cyclopentanecarboxylic acid ((S)-3-{5-[2-(3-hydroxy-pyrrolidin-1-yl)-4,6-dimethyl-pyrimidine-5-carbonyl]-hexahydro-pyrrolo[3,4-c]pyrrol-2-yl}-1-phenyl-propyl)-amide;
tetrahydro-furan-3-carboxylic acid [(S)-3-[5-(2-cyclopropyl-4,6-dimethyl-pyrimidine-5-carbonyl)-hexahydro-pyrrolo[3,4-c]pyrrol-2-yl]-1-(3-fluoro-phenyl)-propyl]-amide;
3,3-difluoro-cyclobutanecarboxylic acid ((S)-3-{5-[2-(1,1-dioxo-1λ⁶-isothiazolidin-2-yl)-4,6-dimethyl-pyrimidine-5-carbonyl]-hexahydro-pyrrolo[3,4-c]pyrrol-2-yl}-1-phenyl-propyl)-amide;
3,3-difluoro-cyclobutanecarboxylic acid ((S)-3-{5-[2-(2-hydroxy-propylamino)-4,6-dimethyl-pyrimidine-5-carbonyl]-hexahydro-pyrrolo[3,4-c]pyrrol-2-yl}-1-phenyl-propyl)-amide;
3,3-difluoro-cyclobutanecarboxylic acid ((S)-3-{5-[2-(4-methanesulfonylamino-piperidin-1-yl)-4, 6-dimethyl-pyrimidine-5-carbonyl]-hexahydro-pyrrolo[3,4-c]pyrrol-2-yl}-1-phenyl-propyl)-amide;
tetrahydro-furan-3-carboxylic acid [(S)-3-[5-(6-ethynyl-2,4-dimethyl-pyridine-3-carbonyl)-hexahydro-pyrrolo[3,4-c]pyrrol-2-yl]-1-(3-fluoro-phenyl)-propyl]-amide;
cyclopentanecarboxylic acid {(S)-3-[5-(3,5-dimethyl-1-pyridazin-3-yl-1H-pyrazole-4-carbonyl)-hexahydro-pyrrolo[3,4-c]pyrrol-2-yl]-1-phenyl-propyl}-amide;
N-{(S)-3-[5-(3,5-dimethyl-1-pyridazin-3-yl-1H-pyrazole-4-carbonyl)-hexahydro-pyrrolo[3,4-c]pyrrol-2-yl]-1-phenyl-propyl}-isobutyramide;
tetrahydro-furan-3-carboxylic acid [(S)-3-{5-[3,5-dimethyl-1-(5-trifluoromethyl-pyridin-2-yl)-1H-pyrazole-4-carbonyl]-hexahydro-pyrrolo[3,4-c]pyrrol-2-yl}-1-(3-fluoro-phenyl)-propyl]-amide;
N—[(S)-3-{5-[3,5-dimethyl-1-(5-trifluoromethyl-pyridin-2-yl)-1H-pyrazole-4-carbonyl]-hexahydro-pyrrolo[3,4-c]pyrrol-2-yl}-1-(3-fluoro-phenyl)-propyl]-acetamide;
cyclopentanecarboxylic acid [(S)-3-[5-(3,5-dimethyl-1-pyrimidin-5-yl-1H-pyrazole-4-carbonyl)-hexahydro-pyrrolo[3,4-c]pyrrol-2-yl]-1-(3-fluoro-phenyl)-propyl]-amide;
[(S)-3-{5-[3,5-dimethyl-1-(5-trifluoromethyl-pyridin-2-yl)-1H-pyrazole-4-carbonyl]-hexahydro-pyrrolo[3,4-c]pyrrol-2-yl}-1-(3-fluoro-phenyl)-propyl]-carbamic acid methyl ester;
N—[(S)-3-[5-(1-cyclohexyl-3,5-dimethyl-1H-pyrazole-4-carbonyl)-hexahydro-pyrrolo[3,4-c]pyrrol-2-yl]-1-(3-fluoro-phenyl)-propyl]-acetamide;
tetrahydro-furan-3-carboxylic acid [(S)-3-[5-(1-cyclohexyl-3,5-dimethyl-1H-pyrazole-4-carbonyl)-hexahydro-pyrrolo[3,4-c]pyrrol-2-yl]-1-(3-fluoro-phenyl)-propyl]-amide;
cyclopentanecarboxylic acid [(S)-3-[5-(1-cyclohexyl-3,5-dimethyl-1H-pyrazole-4-carbonyl)-hexahydro-pyrrolo[3,4-c]pyrrol-2-yl]-1-(3-fluoro-phenyl)-propyl]-amide;
cyclopentanecarboxylic acid [(S)-3-[5-(1-cyclobutyl-3,5-dimethyl-1H-pyrazole-4-carbonyl)-hexahydro-pyrrolo[3,4-c]pyrrol-2-yl]-1-(3-fluoro-phenyl)-propyl]-amide;
N—[(S)-3-[5-(1-cyclobutyl-3,5-dimethyl-1H-pyrazole-4-carbonyl)-hexahydro-pyrrolo[3,4-c]pyrrol-2-yl]-1-(3-fluoro-phenyl)-propyl]-isobutyramide;
tetrahydro-furan-3-carboxylic acid [(S)-3-[5-(1-cyclobutyl-3,5-dimethyl-1H-pyrazole-4-carbonyl)-hexahydro-pyrrolo[3,4-c]pyrrol-2-yl]-1-(3-fluoro-phenyl)-propyl]-amide;
cyclopentanecarboxylic acid [(S)-3-{5-[3,5-dimethyl-1-(6-trifluoromethyl-pyridazin-3-yl)-1H-pyrazole-4-carbonyl]-hexahydro-pyrrolo[3,4-c]pyrrol-2-yl}-1-(3-fluoro-phenyl)-propyl]-amide;
N—[(S)-3-{5-[3,5-dimethyl-1-(6-trifluoromethyl-pyridazin-3-yl)-1H-pyrazole-4-carbonyl]-hexahydro-pyrrolo[3,4-c]pyrrol-2-yl}-1-(3-fluoro-phenyl)-propyl]-acetamide;
N—[(S)-3-{5-[3,5-dimethyl-1-(6-trifluoromethyl-pyridazin-3-yl)-1H-pyrazole-4-carbonyl]-hexahydro-pyrrolo[3,4-c]pyrrol-2-yl}-1-(3-fluoro-phenyl)-propyl]-isobutyramide;
tetrahydro-furan-3-carboxylic acid [(S)-3-{5-[3,5-dimethyl-1-(6-trifluoromethyl-pyridazin-3-yl)-1H-pyrazole-4-carbonyl]-hexahydro-pyrrolo[3,4-c]pyrrol-2-yl}-1-(3-fluoro-phenyl)-propyl]-amide;
1-acetyl-azetidine-3-carboxylic acid [(S)-3-{5-[3,5-dimethyl-1-(6-trifluoromethyl-pyridazin-3-yl)-1H-pyrazole-4-carbonyl]-hexahydro-pyrrolo[3,4-c]pyrrol-2-yl}-1-(3-fluoro-phenyl)-propyl]-amide;
[4-(5-{3-[(1-acetyl-piperidine-4-carbonyl)-(3-chloro-4-methyl-phenyl)-amino]-propyl}-hexahydro-pyrrolo[3,4-c]pyrrole-2-carbonyl)-3,5-dimethyl-pyrazol-1-yl]-acetic acid; TFA salt;
(4-{5-[(S)-3-(cyclopentanecarbonyl-amino)-3-phenyl-propyl]-hexahydro-pyrrolo[3,4-c]pyrrole-2-carbonyl}-3,5-dimethyl-pyrazol-1-yl)-acetic acid;
N—[(S)-3-{5-[3,5-dimethyl-1-(6-methyl-pyridazin-3-yl)-1H-pyrazole-4-carbonyl]-hexahydro-pyrrolo[3,4-c]pyrrol-2-yl}-1-(3-fluoro-phenyl)-propyl]-acetamide;
tetrahydro-furan-3-carboxylic acid [(S)-3-{5-[3,5-dimethyl-1-(6-methyl-pyridazin-3-yl)-1H-pyrazole-4-carbonyl]-hexahydro-pyrrolo[3,4-c]pyrrol-2-yl}-1-(3-fluoro-phenyl)-propyl]-amide;
N—[(S)-3-{5-[3,5-dimethyl-1-(6-methyl-pyridazin-3-yl)-1H-pyrazole-4-carbonyl]-hexahydro-pyrrolo[3,4-c]pyrrol-2-yl}-1-(3-fluoro-phenyl)-propyl]-isobutyramide;
cyclopentanecarboxylic acid [(S)-3-{5-[3,5-dimethyl-1-(6-methyl-pyridazin-3-yl)-1H-pyrazole-4-carbonyl]-hexahydro-pyrrolo[3,4-c]pyrrol-2-yl}-1-(3-fluoro-phenyl)-propyl]-amide;
1-acetyl-azetidine-3-carboxylic acid [(S)-3-{5-[3,5-dimethyl-1-(6-methyl-pyridazin-3-yl)-1H-pyrazole-4-carbonyl]-hexahydro-pyrrolo[3,4-c]pyrrol-2-yl}-1-(3-fluoro-phenyl)-propyl]-amide;
1-acetyl-azetidine-3-carboxylic acid [(S)-3-[5-(3,5-dimethyl-1-pyrazin-2-yl-1H-pyrazole-4-carbonyl)-hexahydro-pyrrolo[3,4-c]pyrrol-2-yl]-1-(3-fluoro-phenyl)-propyl]-amide;
N—[(S)-3-[5-(3,5-dimethyl-1-pyrazin-2-yl-1H-pyrazole-4-carbonyl)-hexahydro-pyrrolo[3,4-c]pyrrol-2-yl]-1-(3-fluoro-phenyl)-propyl]-acetamide;
N—[(S)-3-[5-(3,5-dimethyl-1-pyrazin-2-yl-1H-pyrazole-4-carbonyl)-hexahydro-pyrrolo[3,4-c]pyrrol-2-yl]-1-(3-fluoro-phenyl)-propyl]-isobutyramide;
cyclopentanecarboxylic acid [(S)-3-[5-(3,5-dimethyl-1-pyrazin-2-yl-1H-pyrazole-4-carbonyl)-hexahydro-pyrrolo[3,4-c]pyrrol-2-yl]-1-(3-fluoro-phenyl)-propyl]-amide;
tetrahydro-furan-3-carboxylic acid [(S)-3-[5-(3,5-dimethyl-1-pyrazin-2-yl-1H-pyrazole-4-carbonyl)-hexahydro-pyrrolo[3,4-c]pyrrol-2-yl]-1-(3-fluoro-phenyl)-propyl]-amide;
[5-(5-{3-[(1-acetyl-piperidine-4-carbonyl)-(3-chloro-4-methyl-phenyl)-amino]-propyl}-hexahydro-pyrrolo[3,4-c]pyrrole-2-carbonyl)-4,6-dimethyl-2-oxo-2H-pyridin-1-yl]-acetic acid ethyl ester;
[5-(5-{3-[(1-acetyl-piperidine-4-carbonyl)-(3-chloro-4-methyl-phenyl)-amino]-propyl}-hexahydro-pyrrolo[3,4-c]pyrrole-2-carbonyl)-4,6-dimethyl-2-oxo-2H-pyridin-1-yl]-acetic acid; TFA salt;
cyclopentanecarboxylic acid {(S)-3-[5-(2,4-dimethyl-6-oxo-1,6-dihydro-pyridine-3-carbonyl)-hexahydro-pyrrolo[3,4-c]pyrrol-2-yl]-1-phenyl-propyl}-amide;
[5-(5-{3-[(1-acetyl-piperidine-4-carbonyl)-(3-chloro-4-methyl-phenyl)-amino]-propyl}-hexahydro-pyrrolo[3,4-c]pyrrole-2-carbonyl)-4,6-dimethyl-pyridin-2-yloxy]-acetic acid; TFA salt
1-acetyl-piperidine-4-carboxylic acid (3-chloro-4-methyl-phenyl)-{3-[5-(2,4-dimethyl-6-oxo-1,6-dihydro-pyridine-3-carbonyl)-hexahydro-pyrrolo[3,4-c]pyrrol-2-yl]-propyl}-amide; TFA salt;
2-[5-(5-{3-[(1-acetyl-piperidine-4-carbonyl)-(3-chloro-4-methyl-phenyl)-amino]-propyl}-hexahydro-pyrrolo[3,4-c]pyrrole-2-carbonyl)-4,6-dimethyl-pyridin-2-yloxy]-propionic acid ethyl ester
2-[5-(5-{3-[(1-acetyl-piperidine-4-carbonyl)-(3-chloro-4-methyl-phenyl)-amino]-propyl}-hexahydro-pyrrolo[3,4-c]pyrrole-2-carbonyl)-4,6-dimethyl-pyridin-2-yloxy]-propionic acid;
4,4-difluoro-cyclohexanecarboxylic acid (3-chloro-4-methyl-phenyl)-{3-[5-(2,4-dimethyl-6-oxo-1,6-dihydro-pyridine-3-carbonyl)-hexahydro-pyrrolo[3,4-c]pyrrol-2-yl]-propyl}-amide;
[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-2-oxo-2H-pyridin-1-yl]-acetic acid ethyl ester;
[5-(5-{3-[(3-chloro-4-methyl-phenyl)-(4,4-difluoro-cyclohexanecarbonyl)-amino]-propyl}-hexahydro-pyrrolo[3,4-c]pyrrole-2-carbonyl)-4,6-dimethyl-2-oxo-2H-pyridin-1-yl]-acetic acid;
cyclopentanecarboxylic acid [(S)-3-[5-(2,4-dimethyl-6-oxo-1,6-dihydro-pyridine-3-carbonyl)-hexahydro-pyrrolo[3,4-c]pyrrol-2-yl]-1-(3-fluoro-phenyl)-propyl]-amide;
4,4-difluoro-cyclohexanecarboxylic acid [(S)-3-[5-(2,4-dimethyl-6-oxo-1,6-dihydro-pyridine-3-carbonyl)-hexahydro-pyrrolo[3,4-c]pyrrol-2-yl]-1-(3-fluoro-phenyl)-propyl]-amide;
cyclopentanecarboxylic acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide;
3,3-difluoro-cyclobutanecarboxylic acid [(S)-3-[5-(2,4-dimethyl-6-oxo-1,6-dihydro-pyridine-3-carbonyl)-hexahydro-pyrrolo[3,4-c]pyrrol-2-yl]-1-(3-fluoro-phenyl)-propyl]-amide;
4,4-difluoro-cyclohexanecarboxylic acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide;
3,3-difluoro-cyclobutanecarboxylic acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide;
tetrahydro-furan-3-carboxylic acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide;
3-oxo-cyclobutanecarboxylic acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide;
1-cyclopentanecarbonyl-azetidine-3-carboxylic acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide;
cyclopentanecarboxylic acid {(S)-1-phenyl-3-[5-(1,4,6-trimethyl-2-oxo-1,2-dihydro-pyrimidine-5-carbonyl)-hexahydro-pyrrolo[3,4-c]pyrrol-2-yl]-propyl}-amide;
1-acetyl-piperidine-4-carboxylic acid (3-chloro-4-methyl-phenyl)-{3-[5-(2-pyrrolidin-1-yl-pyridine-3-carbonyl)-hexahydro-pyrrolo[3,4-c]pyrrol-2-yl]-propyl}-amide; TFA salt;
1-acetyl-piperidine-4-carboxylic acid (3-chloro-4-methyl-phenyl)-{3-[5-(2-pyrrolidin-1-yl-pyridine-3-sulfonyl)-hexahydro-pyrrolo[3,4-c]pyrrol-2-yl]-propyl}-amide; TFA salt;
3,3-difluoro-cyclobutanecarboxylic acid ((S)-3-{5-[2,4-dimethyl-6-oxo-1-(2,2,2-trifluoro-ethyl)-1, 6-dihydro-pyridine-3-carbonyl]-hexahydro-pyrrolo[3,4-c]pyrrol-2-yl}-1-phenyl-propyl)-amide;
cyclopentanecarboxylic acid ((S)-3-{5-[2,4-dimethyl-6-oxo-1-(2-oxo-propyl)-1,6-dihydro-pyridine-3-carbonyl]-hexahydro-pyrrolo[3,4-c]pyrrol-2-yl}-1-phenyl-propyl)-amide;
cyclopentanecarboxylic acid ((S)-3-{5-[1-(2-hydroxy-propyl)-2,4-dimethyl-6-oxo-1,6-dihydro-pyridine-3-carbonyl]-hexahydro-pyrrolo[3,4-c]pyrrol-2-yl}-1-phenyl-propyl)-amide;
3,3-difluoro-cyclobutanecarboxylic acid ((S)-3-{5-[2,4-dimethyl-6-(2-oxo-propoxy)-pyridine-3-carbonyl]-hexahydro-pyrrolo[3,4-c]pyrrol-2-yl}-1-phenyl-propyl)-amide;
3-hydroxy-cyclobutanecarboxylic acid ((S)-3-{5-[6-(2-hydroxy-propoxy)-2,4-dimethyl-pyridine-3-carbonyl]-hexahydro-pyrrolo[3,4-c]pyrrol-2-yl}-1-phenyl-propyl)-amide; and,
cyclopentanecarboxylic acid {(S)-3-[5-(5-chloro-2,4-dimethyl-6-oxo-1,6-dihydro-pyridine-3-carbonyl)-hexahydro-pyrrolo[3,4-c]pyrrol-2-yl]-1-phenyl-propyl}-amide.

13. A method for treating or preventing an human immunodeficiency virus (HIV-1) infection, or treating AIDS or ARC, in a patient in need thereof which comprises administering to the patient a therapeutically effective amount of a compound of claim 1.

14. A method according to claim 13 further comprising co-administering at least one compound selected from the group consisting of HIV-1 nucleoside reverse transcriptase inhibitors, HIV-1 non-nucleoside reverse transcriptase inhibitors, HIV-1 protease inhibitors and viral fusion inhibitors.

15. A method according to claim 14 wherein the non-nucleoside reverse transcriptase inhibitor is selected from the group consisting of efavirenz, nevirapine or delavirdine, and/or the nucleoside reverse transcriptase inhibitor is selected from the group consisting of zidovudine, didanosin, zalcitabine, stavudine; lamivudine, abacavir, adefovir and dipivoxil, and/or the protease inhibitor is selected from the group consisting of saquinavir, ritonavir, nelfinavir, indinavir, amprenavir and lopinavir and the viral fusion inhibitor is T-20.

16. A method for treating a mammal with a disease state that is alleviated by a CCR5 receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis which comprises administering to the mammal in need thereof a therapeutically effective amount of a compound of claim 1.

17. A method of claim 16 further comprising co-administering at least one other immune modulator.

18. A method of claim 17 where the mammal is a human.

19. A pharmaceutical composition for treating or preventing an human immunodeficiency virus (HIV-1) infection, or treating AIDS or ARC comprising a compound according to claim 1 admixed with at least one pharmaceutical acceptable carrier, diluent or excipient.

20. A pharmaceutical composition for treating a mammal with a disease state that is alleviated by a CCR5 receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis comprising a compound according to claim 1 admixed with at least one pharmaceutical acceptable carrier, diluent or excipient.
